SEQUENCE LISTING (62 sequences)

SEQ ID NO: 1 - protein, 615 aa, synthetic construct
MENNIQNQCVPYNCLNNPEVEILNEELSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATFEAEYDLERAQK

SEQ ID NO: 2 - DNA, 1848 bp, synthetic construct
  source 1..1848: mol_type = other DNA
atggagaataatattcaaaatcaatgcgtaccttacaattgtttaaataatcctgaagtagaaatattaaatgaagaattaagtactggcagattaccgttagatatatccttatcgcttacacgtttccttttgagtgaatttgttccaggtgtgggagttgcgtttggattatttgatttaatatggggttttataactccttctgattggagcttatttcttttacagattgaacaattgattgagcaaagaatagaaacattggaaaggaaccgggcaattactacattacgagggttagcagatagctatgaaatttatattgaagcactaagagagtgggaagcaaatcctaataatgcacaattaagggaagatgtgcgtattcgatttgctaatacagacgacgctttaataacagcaataaataattttacacttacaagttttgaaatccctcttttatcggtctatgttcaagcggcgaatttacatttatcactattaagagacgctgtatcgtttgggcagggttggggactggatatagctactgttaataatcattataatagattaataaatcttattcatagatatacgaaacattgtttggacacatacaatcaaggattagaaaacttaagaggtactaatactcgacaatgggcaagattcaatcagtttaggagagatttaacacttactgtattagatatcgttgctctttttccgaactacgatgttagaacatatccaattcaaacgtcatcccaattaacaagggaaatttatacaagttcagtaattgaggattctccagtttctgctaatatacctaatggttttaatagggcggaatttggagttagaccgccccatcttatggactttatgaattctttgtttgtaactgcagagactgttagaagtcaaactgtgtggggaggacacttagttagttcacgaaatacggctggtaaccgtataaatttccctagttacggggtcttcaatcctggtggcgccatttggattgcagatgaagatccacgtcctttttatcggacattatcagatcctgtttttgtccgaggaggatttgggaatcctcattatgtactggggcttaggggagtagcatttcaacaaactggtacgaaccacacccgaacatttagaaatagtgggaccatagattctctagatgaaatcccacctcaggataatagtggggcaccttggaatgattatagtcatgtattaaatcatgttacatttgtacgatggccaggtgagatttcaggaagtgattcatggagagctccaatgttttcttggacgcaccgtagtgcaacccctacaaatacaattgatccggagaggattactcaaataccattggtaaaagcacatacacttcagtcaggtactactgttgtaagagggcccgggtttacgggaggagatattcttcgacgaacaagtggaggaccatttgcttatactattgttaatataaatgggcaattaccccaaaggtatcgtgcaagaatacgctatgcctctactacaaatctaagaatttacgtaacggttgcaggtgaacggatttttgctggtcaatttaacaaaacaatggataccggtgacccattaacattccaatcttttagttacgcaactattaatacagcttttacattcccaatgagccagagtagtttcacagtaggtgctgatacttttagttcagggaatgaagtttatatagacagatttgaattgattccagttactgcaacatttgaagcagaatatgatttagaaagagcacaaaagtaa

SEQ ID NO: 3 - protein, 621 aa, synthetic construct
MDNNPNINECIPYNCLSNPEVEVLGGELIETGYTPIDISLSLTQFLLSEFVPGAGFVLGLVDIIWGIFGPSQWDAFPVQIEQLINQRIEEFARNQAISRLEGLSNLYQIYAESFREWEADPTNPALREEMRIQFNDMNSALTTAIPLLAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFSNYDSRRYPIRTVSQLTREIYTNPVLENFDGSFRGMAQRIEQNIRQPHLMDILNSITIYTDVHRGFNYWSGHQITASPVGFSGPEFAFPLFGNAGNAAPPVLVSLTGLGIFRTLSSPLYRRIILGSGPNNQELFVLDGTEFSFASLTTNLPSTIYRQRGTVDSLDVIPPQDNSVPPRAGFSHRLSHVTMLSQAAGAVYTLRAPTFSWQHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQK

SEQ ID NO: 4 - DNA, 1866 bp, synthetic construct
  source 1..1866: mol_type = other DNA
atggataacaatccgaacatcaatgaatgcattccttataattgtttaagtaaccctgaagtagaagtattaggtggagaattaatagaaactggttacaccccaatcgatatttccttgtcgctaacgcaatttcttttgagtgaatttgttcccggtgctggatttgtgttaggactagttgatataatatggggaatttttggtccctctcaatgggacgcatttcttgtacaaattgaacagttaattaaccaaagaatagaagaattcgctaggaaccaagccatttctagattagaaggactaagcaatctttatcaaatttacgcagaatcttttagagagtgggaagcagatcctactaatccagcattaagagaagagatgcgtattcaattcaatgacatgaacagtgcccttacaaccgctattcctcttttggcagttcaaaattatcaagttcctcttttatcagtatatgttcaagctgcaaatttacatttatcagttttgagagatgtttcagtgtttggacaaaggtggggatttgatgccgcgactatcaatagtcgttataatgatttaactaggcttattggcaactatacagattatgctgtgcgctggtacaatacgggattagagcgtgtatggggaccggattctagagattgggtaaggtataatcaatttagaagagagctaacacttactgtattagatatcgttgctctattctcaaattatgatagtcgaaggtatccaattcgaacagtttcccaattaacaagagaaatttatacgaacccagtattagaaaattttgatggtagttttcgtggaatggctcagagaatagaacagaatattaggcaaccacatcttatggatatccttaatagtataaccatttatactgatgtgcatagaggctttaattattggtcagggcatcaaataacagcttctcctgtagggttttcaggaccagaattcgcattccctttatttgggaatgcggggaatgcagctccacccgtacttgtctcattaactggtttggggatttttagaacattatcttcacctttatatagaagaattatacttggttcaggcccaaataatcaggaactgtttgtccttgatggaacggagttttcttttgcctccctaacgaccaacttgccttccactatatatagacaaaggggtacagtcgattcactagatgtaataccgccacaggataatagtgtaccacctcgtgcgggatttagccatcgattgagtcatgttacaatgctgagccaagcagctggagcagtttacaccttgagagctccaacgttttcttggcagcatcgcagtgctgaatttaataatataattccttcatcacaaattacacaaatacctttaacaaaatctactaatcttggctctggaacttctgtcgttaaaggaccaggatttacaggaggagatattcttcgaagaacttcacctggccagatttcaaccttaagagtaaatattactgcaccattatcacaaagatatcgggtaagaattcgctacgcttctactacaaatttacaattccatacatcaattgacggaagacctattaatcagggtaatttttcagcaactatgagtagtgggagtaatttacagtccggaagctttaggactgtaggttttactactccgtttaacttttcaaatggatcaagtgtatttacgttaagtgctcatgtcttcaattcaggcaatgaagtttatatagatcgaattgaatttgttccggcagaagtaacctttgaggcagaatatgatttagaaagagcacaaaagtaa

SEQ ID NO: 5 - protein, 1355 aa, synthetic construct
MENGSGARKESEEKKEVKKGKPNVLSRLFMCWVCPVLVGGNRRDVEERDLIPPPSAKYKSESLGDKFERYWLEELGLATQRGVAPSLWRALRRAFWLSYMPGALLLLGNAIPRTIQPLLFTRLLSYWSADSTMTRLEAGYWAMGMLLCNFLAMVCHHHNTLFVGRFGMKVRIACCSLLYRKLLRLNQRSLQSTAAGKLVNLMSNDVARFDYAFMFLHYFWMIPLQSAAVLYFMFRAAGWAPIVGLFSVMLLILPIQAGLTKLTAVYRRETAQRTDKRIKLMSEIINGIQVIKMYAWEIPFQKVVGSSRAHEVEALKRASFVQGTFGGFMLFTERTSLFLTVMTLVLTGSMATATTVYPIQQYFSIIQSNLALILPIAIAQLTEMLVSLERLQEFLMLDEREDLSVMPGGQADTAPVAFKYTKETTAPAYIVSKRYSKKEDDTGLAAELVERKSTSEFAVELNDVSASWGGEGDKDQHTLRGVSLRVRRGKLAAIIGPVGSGKSSLLQVLLKELPVSSGTVGVHGQISYACQESWLFSATVRDNILFGLPYDSKKYKKVCDACCLQPDFKQFPYGDLSLVGERGVSLSGGQRARINLARAVYRDADIYIFDDPLSAVDANVGRQLFEGCINGYLRGRTRVLVTHQIHFLKAADYIVILNEGAIENMGTYDDLTKLENSLLLPKQQEGSGDDSKDELAIPNAANKPIMERGVSVISVKSEDNGEARKEQVQAAEERASGNLKWEVFARYLVSVDSWAIVALTLTAMLITQGAASSTDYWLSFWTNQVDGYIQDLPDGEEPDPSLGTQTGILLTGQYVYIYGALVLTIIVMSFMRLFGFVTMTMRAAANIHDLMFRNLIRATMRFFDTNPSGRVLNRFSKDMGGMDELLPRSILQAFQMYLSMASVLTLNAVSLPWTLIPTVLLLGLFIRYLKWYLNAAQSVKRLEGTTKSPVFGMIGSTLSGMSTIRSSDSQDRLIKNFDDCQNLHTSAFHTYIGGATAFGFYLDMICLVYLASILSIFILIDFADVIPVGSVGLAVSQSMVLTVLLQLAARFTSDFLAQMTAVERVLEYTKLPHEENINDGPTQPPKTWPAEGNIKFENVFLTYSLEDPPVLKNINFEIQSGWKVGVVGRTGAGKSSLISALFRLTNLDGSIKIDGIDTIGIAKQELRAKISIIPQEPVLFSATLRYNLDPFDLYSDDDIWRALEQVELKDVVPALDYKVSEGGSNFSVGQRQLLCLARAVLRSNKILVMDEATANVDPQTDALIQSTIRRQFAACTVLTIAHRLNTVMDSDRVLVMDKGEVVEFDHPYTLLSAPGSHLNFMVEETGDNMSKALYDMAKKKYFDDHPQDYKDDDDK

SEQ ID NO: 6 - DNA, 4068 bp, synthetic construct
  source 1..4068: mol_type = other DNA
atggaaaacggaagcggagcccggaaagagtcggaagagaagaaagaggtgaagaaaggcaagcccaacgtgctgtcccggctgttcatgtgctgggtgtgccccgtgctggtgggcggcaaccggcgcgacgtggaggagcgggacctcataccgccgcccagcgccaagtacaagtccgagtctctcggagacaagtttgaaaggtactggctagaagagctgggtctggcgacgcagcgcggggtggcgccgtcgttgtggcgtgcgctgcgccgcgccttctggctgtcctacatgccgggcgcgctgctgctgctcgggaacgccatacccaggaccatccagccgctgctattcacgcgactgctctcctattggtcggcggacagcaccatgacccgactggaggccggctattgggccatgggcatgctgctgtgcaacttcctggccatggtgtgccaccaccacaacacgctattcgtcggccgcttcggcatgaaagtcaggattgcttgctgctcgctgctctatcggaagctgctccgtctaaaccagaggtccctgcaaagcacggcagcgggcaagctggtgaacctgatgtcgaacgacgtggcgcgcttcgactacgccttcatgttcctgcactacttctggatgatcccgctgcagtcggccgccgtgctgtacttcatgttccgcgccgccggctgggcgcccatcgtgggcctgttctctgtcatgctgctcatactgcctatacaagccggcctcaccaagctcacagctgtctacaggagggagactgcacaaaggacagataagagaattaaactaatgagcgaaattatcaatggtattcaggtgataaagatgtacgcgtgggagatccccttccagaaggtggtgggttcctcccgcgcgcacgaggtggaggcgttgaagcgggcgtccttcgtgcagggcaccttcggggggttcatgctgttcacggagcgcacctcgctcttcctcaccgtcatgacgctcgtgctgactgggagcatggctactgctactacggtgtatccaatccaacaatactttagtataatccaatccaatttggcgctcatcctccctatcgccatcgctcaactcactgagatgttggtctcattggaaagacttcaggagtttctgatgttagacgagagggaagacctgtcggtgatgccgggcgggcaggcggacaccgcgcccgtggccttcaagtacacgaaggagaccacggcgcccgcctacatcgtgtccaagaggtactccaagaaggaggacgacaccggcctggctgcggagctggtggagcgcaaatccacgagcgagttcgcggtcgagctgaacgacgtgagtgcgtcgtgggggggtgagggggacaaggaccagcacacgctgcggggggtgagcctgcgcgtgcgccgcggcaagctggccgccatcatcggacccgtcggctcggggaagtcgtcgctactccaagtgttgctgaaggagcttccggtgtcttccggcaccgtgggtgtgcacggccagatctcgtacgcgtgtcaggagtcctggctcttctccgccacggttcgggataacatcctcttcggactgccgtatgattcgaagaaatataagaaggtgtgcgacgcgtgctgcctgcagcccgacttcaagcagttcccgtacggagacctgtcgctggtgggcgagcgcggcgtgtcgctgtccggggggcagcgcgcccggatcaacctcgcgcgcgccgtgtaccgggacgccgacatctacatattcgatgaccccttatcggcggtggacgcgaatgtgggtcggcagctgttcgagggctgcatcaacggctacctgcgcggccgcacgcgcgtgctcgtcacgcatcagatacacttcctcaaggccgcagactacatagtcatactcaacgagggtgccattgaaaatatgggcacgtacgatgatttaacgaagctggagaattcgctgctgctgcccaagcaacaggagggttccggcgatgacagtaaggatgaactagctattccgaatgcagcaaacaagccgataatggaacggggggtatcagtgatctcagtgaagagcgaagacaacggtgaggctcgcaaggaacaggtgcaggcggcggaggagcgcgcctccgggaacctcaagtgggaggtgttcgcgaggtacctggtctccgtggactcctgggccatcgtggcgctcacgctcaccgcgatgctcatcacgcagggggcggcgtcgtccaccgactactggcttagcttctggaccaatcaagttgatggatacatacaagacctgccagatggggaggagccagatccaagtctcggcacgcaaacgggcatcctgctgacgggccagtacgtgtacatctacggcgcgctggtgctcaccatcatcgtgatgtccttcatgcggctgttcggcttcgtgaccatgaccatgcgcgccgccgccaacatccacgacctcatgttccgaaacctcatccgcgccaccatgcgcttctttgacaccaatccttcaggtcgcgtgctaaaccggttctccaaagacatgggcggcatggacgagctgctgcccaggtccatcctgcaggccttccagatgtacctgtccatggccagcgtgctcacgctgaacgccgtctccctgccctggaccctcatacccacggtgctgctgctgggactcttcatcaggtacctcaagtggtacctgaacgctgcgcagtctgtgaagaggctggagggcacaaccaagagtccagtgtttggaatgatcggctctactctatcgggaatgtcgacaatcagaagttcagactcacaggacagactaatcaaaaacttcgacgactgccagaatctccatacttctgccttccacacttacatcgggggagccacggccttcggattctatttggatatgatatgcctcgtctacctcgcgtctatattatcaattttcatccttatcgacttcgcggacgtgatcccggtgggcagcgtggggctggcggtgagccagtccatggtgctgacggtgctgctgcagctggcggcgcggttcaccagcgacttcctggcgcagatgaccgccgtggagcgcgtgctcgagtacactaagctgccgcacgaggagaatattaatgatggccctacgcagccgcccaagacatggcctgctgaagggaacatcaaatttgaaaacgtcttcctgacttactcgttggaggacccacccgtgctcaaaaatattaacttcgagatccaaagcggttggaaggtgggcgtggtcggcaggaccggggccgggaagtcatctctcatatctgcccttttccgattgaccaatttagacggaagcattaaaattgatgggatcgatacaataggaatagctaagcaggagctcagggcgaagatttcaattataccgcaagagcctgttctgttctcggccacgctgcgctacaacctggaccccttcgacttgtacagtgacgacgatatttggagagctttggaacaggtggagttgaaagacgtggttccggcgctcgactacaaagtgtcggaaggaggtagcaatttctccgtgggacagcggcagctgctgtgcctggcgcgcgccgtgctgcgctccaacaagatactagtcatggacgaggccaccgccaatgtcgacccgcagacggacgcgctgatccagtcgacgatccggcggcagttcgcggcctgcaccgtgctcaccatcgcgcatcgtctcaacaccgtcatggactccgaccgagtgctcgtcatggacaagggtgaagtggtcgagttcgaccacccgtacacgctgctgtcagcccccggcagccacctcaacttcatggtggaggagaccggagacaacatgagcaaggccctttatgacatggccaagaagaaatacttcgacgaccatcctcaagactacaaagacgatgacgacaagtag

SEQ ID NO: 7 - protein, 1350 aa, synthetic construct
MNSDGRAGENSSAETRRKPHKPNILSRIFLWWMCPVLVKGNQRDTVEDDLIIPKKSFNSENQGEYLERYWLQEYEAAIKEKREPSLWTALRKAYWLGYMPGAIYLIIISVFRIIQPLVFAELLSYWSVEATITRLEASYYALALLGINFINMMCQHHNSLFVARFGLKVKVACSSLVYRKLLRMDQVALGDVSGGKLVNLLSNDVARFDYAFMFLHYLWVVPVQAAVVLYFLYISAGYAPFVGFFGVVILILPIQAGLTKLTSVVRRETAQRTDRRIKLMTEIINGIQVIKMYAWEKPFQAIVKVARNFEMIALRKSIFIRSVFLGFMLFTERSIIFITCLTFLLTGNLVTATLIYPIQQYISIIQINLTMILPLAIASLSEMLVSLERIQNFLVKDEREDVQITPKSYGDDNRLIFNNKASLGPQNEIIPKKYLATDGQLASTLTNEPVLSTDPAVCDYPIELSKVDATWSSSTDTSEMTLRNITLRIGRGKLCAIIGPVGSGKSSILQVLLKELPVCGGSLRINGRLSYACQESWLFPATVRENILFGLPYDSQKYHEVCKACSLLPDFKQFPYGDLSLVGERGVSLSGGQRARINLARAVYREADIYLLDDPLSAVDANVGRQLFDGCIKGYLRGRTCVLVTHQIHYLKAADIIVILNEGAIENVGSYDDLVNTGTEFSKLLTNQESNDNENGPEKNFLRAISKISTKSVEDPDNEKVQVEEEEKRAKGNLKFSVLYKYLSAVKSWFLVFLMVVTLVITQGCATFIDYWLSFWTNQVDEYEQSLAEGEEPSTSLDTQAGAFTLGVYLWTYGGVILILIVISHVRILTFVITTMRASSNFHDTVYKKLIITVMRFFDMNPSGRVLNRFSKDMGAMDEFLPRSLFETVQMYLTLCSILILNAIALPWTLIPTAVLLILFFVLLKWYLNAAQAVKRLEGTTKSPVLGMINSTLTGLSTIRSSNSQGRLLQMFDNAQNLHTSAFYTFVGGSTAFGLYLDALCLVYLGVILTIFLVIDFSTLIPVGSVGLAVSQSMVLTMMLQMAARFTADFLGQMTAVERVLEYTELPMEENMYDGSQLPKDWPTHGRIEFQNLFLNYSQEDPPVLKDLNFVIENGWKVGVVGRTGAGKSSMISALFRLYELQGHIRIDGIDTNIIAKTELRSKISIIPQEPILFSASVRYNLDPFDSYSDDEIWRALEQVELKEVIPALDYKVSEGGSNFSVGQRQLVCLARAVLRSNKILVMDEATANVDPQTDALIQTTIRREFASCTVITIAHRLNTIMDSDRVLVMDKGVVAEYDTPYALLSDPNSIFSSMVRETGDTMSKVLFRVAEDKHLGRNTEKDYKDDDDK

SEQ ID NO: 8 - DNA, 4053 bp, synthetic construct
  source 1..4053: mol_type = other DNA
atgaatagtgatgggagagccggagaaaattctagtgctgagacgcgaaggaagccccataagccgaacattttgtctcgtatatttctatggtggatgtgccccgtactcgttaaaggaaatcaaagagacactgtcgaggatgacctcataattccgaaaaagtcatttaattctgaaaatcaaggagagtatttagaaaggtattggctccaagaatatgaagccgcgataaaggagaaaagggagccgtcattgtggacggctttacgaaaagcttactggttgggatacatgccgggagctatctacttgataatcatatctgtcttcaggataattcagccgctagtattcgccgaactgctttcctactggtcagtagaagcaactataactcgactagaagcaagctattatgctctagcacttttaggtataaacttcattaatatgatgtgtcaacatcacaattcattgtttgtagcgcgctttggactcaaggttaaagtggcttgctcatctttagtatatagaaagttgttgcgcatggaccaagtagccttaggagatgtaagcggagggaaactggtgaacctgttatctaatgatgttgctagattcgactacgcattcatgtttttgcactacctctgggtggtaccagttcaagcggctgtagtactatatttcttatatatttctgcgggatacgcaccgttcgtcggcttctttggggttgtgattttaattctacctatacaagctggtctcacaaaattgacctctgtggtcagaagagaaactgctcagagaaccgaccgaagaataaaattgatgacggaaattattaatggcattcaggttattaaaatgtacgcatgggagaaaccgttccaagcaatcgtcaaggtggcaagaaacttcgaaatgatagcactcaggaagtctatattcattcggagtgttttccttggtttcatgctgttcactgaaagaagtataatatttataacatgtttgacatttttacttacgggtaaccttgttacagctactctgatttaccctattcaacaatacatcagcattattcaaatcaatttgactatgatactgcctcttgccattgcaagtttgtctgaaatgctggtctcgttggaacgcattcaaaatttcttggtgaaagatgaaagagaggacgtccaaataacaccaaaatcttatggagatgataatagattgattttcaacaacaaggcatcgcttggccctcaaaacgaaattatcccaaagaaatatttagccaccgatggccaacttgcctcaacattaacgaatgaaccagtgctttcaactgacccagccgtgtgtgactatcccatagaattatctaaagtagacgccacatggtcgtcatcaacagatacttcagaaatgacgctaaggaatattactctcagaataggcagaggcaaactttgtgctataattggacctgttggatcaggaaagtcatccattttgcaagttcttctgaaagaactgccggtttgcggtggtagcctgagaataaatgggaggctttcctacgcgtgtcaagagtcttggctgttccctgctactgtgagagaaaacattttgtttggtttgccgtacgactcacagaaatatcacgaggtgtgtaaagcttgttcgttgcttcctgatttcaaacaatttccgtacggagacctctccttagttggagagcgaggagtatccttgtcagggggacagcgtgccagaattaatctcgctagggctgtttaccgagaggccgacatttacttgctagatgatcctctctcagcagtggatgccaacgttggcaggcagctgtttgacggttgtatcaaaggatatctgcggggtcgaacttgtgtccttgtaacccatcaaatccactatcttaaagcagctgacattattgtcatactgaacgagggagcaattgaaaacgttggtagctacgatgatttggtcaacaccggaacagagttttcgaagctgttgactaatcaagaaagcaacgataatgagaatggtccggaaaagaatttcttacgggctatatcgaaaatatctactaagagtgttgaagatccagataacgagaaggtacaagtggaggaagaagagaaacgtgccaaaggaaatttgaaattctctgttttatacaagtatttgtctgcagtgaaatcatggtttctagtatttctgatggtagtcacattagttatcacacaaggatgtgcgacgttcatagattattggctgagtttttggacaaatcaagttgatgagtatgaacaatctttagctgagggagaagaaccgagcacaagtttggacacgcaagccggagcgttcactttaggagtatacctttggacttatggcggagtcatcttaatcctcatagtgatcagtcacgtcagaattttaactttcgtcattacgactatgagagcttcatctaattttcatgataccgtttataaaaagttgattatcacagttatgcgattctttgacatgaatccctcaggtcgtgtgttgaatcgtttttcaaaggatatgggtgcgatggacgaattccttccgaggagtttatttgaaactgttcagatgtatttaacattatgcagtatacttattttaaatgctatagcattaccgtggacgttgatcccaacagcggtacttttgattctcttcttcgttctcctgaaatggtacttaaatgctgctcaagctgtaaaaagattagaaggcacaaccaagagtccagtactgggcatgatcaactcaaccctcacaggtctgtctacgatccgaagttctaactcacaaggacgactgctacaaatgtttgataacgcacagaatctccacacgagtgcattttatacgtttgttggtggatcaactgcatttggtttatatctggatgcgctttgcctagtttatttgggagtgatattgacgatcttccttgtaatcgacttcagtactttaattcccgtcggtagcgtgggcttggctgtgagtcagtctatggtgttaacgatgatgctgcaaatggcggctcggttcacagccgatttcctcggacaaatgacggctgtcgagagagttctcgaatataccgagcttccgatggaagaaaacatgtatgatggatcccaacttcccaaggactggcctacacacggtcgtattgaatttcaaaatctatttttgaactattctcaagaagatccaccagtactcaaagatttaaactttgtaattgagaatggttggaaggttggagtagtcggtcgaaccggtgccggcaagtcttctatgatatccgcactgttcaggctgtacgaactgcaaggacacatcagaatagacggaatcgataccaacatcatcgctaaaacggagctaagatcgaagatttctatcattccacaagaaccgattttgttctctgcatcggtgcgatacaatttggatcccttcgacagttatagcgacgacgaaatttggagagctttggaacaggttgaactgaaagaggtaattccagcgctcgattataaagtgtcagaaggtggcagcaacttctccgtcggtcagcgacaactggtatgtttggctcgagctgttttgcgttctaacaaaatccttgtaatggacgaggcgacagccaacgttgatcctcaaactgacgcgttgattcagacgacgatacgtcgcgagtttgcgtcatgtacggtgatcacgatcgcgcaccgactcaacacgatcatggattctgatcgcgtcctggtcatggacaaaggagttgttgccgaatacgatactccttatgctctgctctccgaccccaattcgatcttcagctcgatggtgcgcgaaacaggagatacaatgagcaaagtcctcttccgagtggctgaagataaacatcttggtagaaatacagaaaaagactacaaagacgatgacgacaagtag

SEQ ID NO: 9 - protein, 1356 aa, synthetic construct
MDKSNKNTAANGNGGQRAGEPKKRVRKKPNILSRIFVWWIFPVLITGNKRDVEEDDLIVPSKKFNSERQGEYFERYWFEEVAIAEREDRDPSLWKAMRRAYWLQYMPGAIFVLLISGLRTAQPLLFSQLLSYWSVDSEMSQQDAGLYALAMLGINFITMMCTHHNNLFVMRFSMKVKIAASSLLFRKLLRMSQVSVGDVAGGKLVNLLSNDVARFDYAFMFLHYLWVVPLQVGVVLYFVYDAAGWAPYVGLFGVIILIMPLQAGLTKLTGVVRRMTAKRTDKRIKLMSEIINGIQVIKMYAWEKPFQLVVKAARAYEMSALRKSIFIRSMFLGFMLFTERSVMFLTVLTLALTGNMISATLIYPIQQYFGIITMNVTLILPMAFASFSEMLISLERIQGFLLLDERSDIQITPKVVNGAGSKLFNNSKKEGGLETGIVLPTKYSPTEASIARPVQDEPNMADYPVQLNKVNATWADLNDNKEMTLKNISLRVRKNKLCAVIGPVGSGKTSLLQLLLRELPVTSGNLSISGTVSYASQEPWLFPATVRENILFGLEYNVAKYKEVCKVCSLLPDFKQFPYGDLSLVGERGVSLSGGQRARINLARAVYREADIYLLDDPLSAVDANVGRQLFDGCIKGYLSGKTCILVTHQIHYLKAADFIVVLNEGSVENMGSYDELMKTGTEFSMLLSDQASEGSDTDKKERPAMMQGISKMSVKSDDEEGEEKVQVLEAEERQSGSLKWDVLGRYMKSVNSWCMVVMAFLVLVITQGAATTTDYWLSFWTNQVDGYIQTLPEGESPNPELDTQVGLLTTGQYLIVHGSVVLAIIILTQVRILSFVVMTMRASENLHNTIYEKLIVAVMRFFDTNPSGRVLNRFSKDMGAMDELLPRSMLETVQMYLSLASVLVLNAIALPWTLIPTTVLMFIFVFLLKWYINAAQAVKRLEGTTKSPVFGMINSTISGLSTIRSSNSQDRLLNSFDDAQNLHTSAFYTFLGGSTAFGLYLDTLCLIYLGIIMSIFILGDFGELIPVGSVGLAVSQSMVLTMMLQMAAKFTADFLGQMTAVERVLEYTKLPTEENMETGPTTPPKGWPSAGEVTFSNVYLKYSPDDPPVLKDLNFAIKSGWKVGVVGRTGAGKSSLISALFRLSDITGSIKIDGLDTQGIAKKLLRSKISIIPQEPVLFSASLRYNLDPFDNYNDEDIWRALEQVELKESIPALDYKVSEGGTNFSMGQRQLVCLARAILRSNKILIMDEATANVDPQTDALIQKTIRKQFANCTVLTIAHRLNTIMDSDRVLVMDQGVAAEFDHPYILLSNPNSKFSSMVKETGDNMSRILFEVAKTKYESDSKTADYKDDDDK

SEQ ID NO: 10 - DNA, 4071 bp, synthetic construct
  source 1..4071: mol_type = other DNA
atggacaaatctaataaaaataccgcggcaaatggcaatggtggtcaacgtgcgggcgaaccaaagaagagagtaagaaaaaaaccaaacatattgtcacgtatatttgtttggtggatatttcctgtgctcattactggtaacaagcgagacgtagaagaagacgatttaattgttccaagtaaaaaatttaactctgaaagacaaggagaatattttgaaagatattggttcgaggaggtagcaattgctgagagagaagacagagatccttcactatggaaagcgatgcggcgcgcttactggctgcaatacatgccgggagctatcttcgtgctgctcatttctggattaaggacagctcagccattgctgttctctcagctcctgtcctactggtcggtggacagtgagatgtcgcagcaggatgctggtctctatgccctggccatgttggggattaactttatcaccatgatgtgcacgcaccacaacaacttgttcgtcatgcgattcagtatgaaagtcaaaatcgccgcctcgtcactcttatttagaaagttgctccgcatgagtcaagtgtcagtgggcgatgtcgccggtggaaagttagtgaacttgctgtccaacgatgtcgcgaggtttgactatgcgttcatgttcctacactacctgtgggtggtaccgctgcaagtcggagttgtcctgtacttcgtgtacgatgctgctgggtgggcgccatatgtcggactctttggagtgattatattaatcatgccacttcaagccggtctaactaaactcacgggtgttgtgagacgaatgacagctaagaggaccgacaaaagaattaagcttatgagtgaaattatcaacggtatacaggttatcaaaatgtacgcttgggagaaacccttccagctagtggtgaaggcggctcgtgcgtatgaaatgagtgctcttaggaaatctatcttcatcaggagcatgttcctcgggtttatgttgttcaccgagcgaagtgtcatgtttctgactgtgctgactctagcgctgactggaaacatgattagtgccactctgatttatcccatccaacagtacttcggtattatcacaatgaacgttacccttattttaccgatggcgtttgcaagtttctctgagatgttgatatcgttggaacgtattcaaggattcctacttttggacgagcgttcagacattcaaattacaccaaaagtggtgaatggtgctggaagcaaattgttcaacaattccaagaaggagggaggcctcgagactggcattgtgttgccaacaaaatactcacctaccgaagcaagtattgccagacccgtgcaggatgagcctaacatggccgactatccggtgcaacttaacaaagtgaatgcaacatgggcggacctcaatgataacaaagaaatgacactcaagaatatatctttacgtgttcgcaaaaacaaactatgcgctgttattggacctgtgggatcaggaaagacctctcttcttcagctccttttgagggagttgcccgtgactagcggcaacctcagcatatctggtaccgtatcctatgctagtcaggagccttggctgttccctgcgaccgtgcgggagaacattctgtttggtttggaatacaatgtcgccaaatataaagaggtttgtaaagtctgctcattactgccagactttaagcagttcccgtacggtgacttgtctctggttggggagcgaggtgtgtcactgtctggtggtcagagggccaggatcaatttggccagagctgtctaccgcgaggctgatatttacttgcttgacgatcctctatcggctgtagacgcaaacgtcggcaggcaactattcgacggctgcatcaaaggatacctcagtggcaaaacatgcatcctggtcacccaccaaattcactaccttaaagctgcagactttattgtagtcctaaacgagggttccgtcgaaaatatgggctcgtatgatgaactaatgaaaactggaacggaattctcgatgctgctctctgaccaagctagtgaaggctctgacactgacaaaaaagaacggccagcaatgatgcaaggaatatcaaagatgtcagtcaagagtgacgatgaggaaggtgaggagaaggttcaagtattggaggccgaagagagacagtcgggcagcctgaagtgggatgtgcttgggaggtacatgaagtcggtcaactcctggtgcatggtggtaatggcattcctcgtgttggtgatcacgcagggtgctgccaccactaccgactactggcttagtttctggactaatcaagtggatggatacatacaaactttacccgaaggagaaagcccaaatcctgagttggatactcaagtcggtctattaaccacgggacagtacctcattgtgcacggcagcgtagtactagccattataatattgacgcaagtcagaatactttccttcgtagtgatgactatgcgagcttcggaaaatcttcataacaccatttacgagaaattgatagtggctgtaatgagatttttcgataccaatccatcgggtcgtgtattgaacagattctcaaaagatatgggtgctatggatgagctgttgccgcgaagcatgttggagactgttcagatgtacctgtctctagccagtgtccttgtgctgaacgccatagccttaccttggacattgatacccaccacagtgttgatgtttatatttgtattcctattgaagtggtacattaacgctgctcaagctgtgaaacgattggaaggaactacaaagagtccagtgtttggaatgatcaactctactatatctggactctccaccataagaagctccaactctcaggaccgacttctaaactcatttgatgatgcacagaatctccatactagcgctttctacacgtttttgggtggttcaaccgcatttggtctgtacctggacaccctctgtttaatctacctcggaattatcatgtccatcttcattcttggtgatttcggtgagctgataccggttggtagcgtcggtctggccgtcagtcagtccatggtgctcaccatgatgttgcaaatggccgccaagttcactgccgacttcctgggacagatgacagccgtcgagagggtcctggagtacaccaagctacctaccgaagaaaacatggagactggaccgacaacgccaccaaagggctggccaagtgctggagaggtgacgttctcaaacgtgtacctcaagtattctcctgacgacccaccagtactgaaggacttgaactttgcaatcaagagtggatggaaggtcggagtagttggtagaactggtgctggcaagtcctcgttgatatcagctctgttccggctcagcgacattacaggcagtatcaaaattgatggcctggatactcaaggaattgccaagaagcttttgagatcaaaaatatcaataattccgcaagagccagtgttgttctctgcctcgctgcgttacaatctggacccattcgacaactacaacgatgaagatatttggagagcattggaacaggtggaactaaaggagagtataccagccctcgattacaaagtgtcggaaggcggcaccaacttctcaatgggacaacgtcagctggtgtgcctggcgcgcgccatcctccgctcaaataaaattctcatcatggacgaagccaccgctaacgtcgatcctcagacggatgctttgatccagaaaacaattcgtaaacaatttgccaactgcaccgtgctcacgatcgcgcatcgactgaacaccattatggattcagatcgggtactagtcatggaccagggagtggccgcggagttcgaccacccctacatcttgctgtccaaccccaacagcaagttctcctcaatggtgaaggaaacaggcgacaacatgtccagaatcttgttcgaagtagccaaaacaaaatatgaaagtgattccaaaaccgctgactacaaagacgatgacgacaagtag

SEQ ID NO: 11 - protein, 1354 aa, synthetic construct
MDKSNKNTAANGNGGQRAGEPKERVRKKPNILSRIFVWWIFPVLITGNKRDVEEDDLIVPSKKFNSERQGEYFERYWFEEVAIAEREDRDPSLWKAMRRAYWLQYMPGAIFVLLISGLRTAQPLLFSQLLSYWSVDSEMSQQDAGLYALAMLGINFITMMCTHHNNLFVMRFSMKVKIAASSLLFRKLLRMSQVSVGDVAGGKLVNLLSNDVARFDYAFMFLHYLWVVPLQVGVVLYFVYDAAGWAPYVGLFGVIILIMPLQAGLTKLTGVVRRMTAKRTDKRIKLMSEIINGIQVIKMYAWEKPFQLVVKAARAYEMSALRKSIFIRSMFLGFMLFTERSVMFLTVLTLALTGNMISATLIYPIQQYFGIITMNVTLILPMAFASFSEMLISLERIQGFLLLDERSDIQITPKVVNGAGSKLFNNSKKEGGLETGIVLPTKYSPTEANMARPMQDEPNMADYPVQLNKVNATWADLNDNKEMTLKNISSRVRKNKLCAVIGPVGSGKTSLLQLLLRELPVTSGNLSISGTVSYASQEPWLFPATVRENILFGLEYNVAKYKEVCKVCSLLPDFKQFPYGDLSLVGERGVSLSGGQRARINLARAVYREADIYLLDDPLSAVDANVGRQLFDGCIKGYLSGKTCILVTHQIHYLKAADFIVVLNEGSVENMGSYDELMKTGTEFSMLLSDQASEGSDTDKKERPAMMRGISKMSVKSDDEEGEEKVQVLEAEERQSGSLKWDVLGRYMKSVNSWCMVVMAFLVLVITQGAATTTDYWLSFWTNQVDIQTLPEGESKNPELNTQVGLLTTGQYLIVHGSVVLAIIILTQVRILSFVVMTMRASENLHNTIYEKLIVAVMRFFDTNPSGRVLNRFSKDMGAMDELLPRSMLETVQMYLSLASVLVLNAIALPWTLIPTTVLMFIFVFLLKWYINAAQAVKRLEGTTKSPVFGMINSTISGLSTIRSSNSQDRLLNSFDDAQNLHTSAFYTFLGGSTAFGLYLDTLCLIYLGIIMSIFILGDFGELIPVGSVGLAVSQSMVLTMMLQMAAKFTADFLGQMTAVERVLEYTKLPTEENMETGPTTPPKDWPSAGEVTFSNVYLKYSPDDPPVLKDLNFAIKSGWKVGVVGRTGAGKSSLISALFRLSDITGSIKIDGLDTQGIAKKLLRSKISIIPQEPVLFSASLRYNLDPFDNYNDEDIWRALEQVELKESIPALDYKVSEGGTNFSMGQRQLVCLARAILRSNKILIMDEATANVDPQTDALIQKTIRKQFATCTVLTIAHRLNTIMDSDRVLVMDQGVAAEFDHPYILLSNPNSKFSSMVKETGDNMSRILFEVAKTKYESDSKTADYKDDDDK

SEQ ID NO: 12 - DNA, 4065 bp, synthetic construct
  source 1..4065: mol_type = other DNA
atggacaaatctaataaaaataccgcggcaaatggcaatggtggtcaacgtgcgggcgaaccaaaggagagagtaagaaaaaaaccaaacatattatcacgtatatttgtttggtggatattccctgtgctcatcactggcaacaagcgagatgtagaagaagacgatctaattgttccgagtaaaaaattcaattctgaaagacaaggagaatattttgaaagatattggttcgaggaggtagcaattgctgagagagaagaccgagatccttcgttatggaaagcgatgcgccgcgcttactggctgcaatacatgccgggagctatcttcgtgctgctcatttctggattaaggacagctcagccattgctgttctctcagctcctgtcctactggtcggtggacagcgagatgtcgcagcaggatgctggtctctatgccctggccatgctggggatcaacttcatcaccatgatgtgcacgcaccacaacaacttgttcgttatgcggttcagtatgaaagtcaaaatcgccgcctcgtcactcttatttagaaagttgctccgcatgagtcaagtgtcagtgggcgatgtcgctggtggaaagttagtgaacttgctgtctaacgatgtcgcgaggtttgactacgcgttcatgttcctgcactacctgtgggtggtaccgctgcaagtcggagtcgtcctgtacttcgtgtacgatgctgctgggtgggcgccatatgtcggtctctttggagtcatcatcttaatcatgccacttcaagccggtctaactaaactcacgggtgttgtgagacggatgacagctaagaggactgacaaaagaattaagcttatgagtgaaattatcaacggtatacaggtcatcaaaatgtacgcttgggagaaacccttccagctagtggtgaaggcggctcgtgcgtatgaaatgagtgctcttaggaagtctatcttcatcaggagcatgttcctcgggtttatgttgttcaccgagcgaagtgtcatgtttctgactgtgctgactctagcgttgactggaaatatgattagtgccactctgatttatcccatccaacagtacttcggtattatcacaatgaacgttaccctcattttaccgatggcgtttgcaagtttctctgagatgttgatatccttggaacgtattcagggattccttcttttggacgagcgttcagacattcaaattacaccaaaagtggtgaatggtgctggaagtaaattgttcaacaattccaagaaggagggaggcctcgagactggcattgtgctgccaacaaaatactcacctactgaagcgaatatggccagacccatgcaggatgagcctaacatggctgactatcctgtgcaacttaacaaagttaatgcaacatgggcggacctcaatgataacaaagagatgacacttaagaatatatcttcacgtgttcgcaaaaacaaactatgcgctgttattggacctgtgggatcaggaaagacctctcttcttcagctccttttgagagaattgcctgtaactagtggtaacctcagcatatctggtaccgtatcctacgctagtcaggagccgtggctgttccctgcgaccgtgcgggagaacattctgtttggtttggaatacaatgtcgccaaatataaagaggtttgtaaagtctgctcattactgccagactttaagcagttcccgtacggtgacttgtctctggttggagaacgaggtgtgtccctgtctggtggtcagagggccaggatcaatttggccagagctgtttaccgcgaggctgatatttacttgcttgacgatcctctatcggctgtagatgcaaacgtcggcaggcaactattcgacggctgcatcaaaggatacctaagcggcaagacatgcatcctggtcacccatcagattcactaccttaaagctgcagactttatcgtagtcctaaacgagggttccgtcgaaaatatgggctcgtatgatgaactaatgaaaactggaacggaattctcgatgctgctctctgaccaagctagtgaaggctctgacactgacaaaaaagaacggccagcaatgatgcgaggaatatcaaagatgtcagtcaagagtgacgatgaggaaggtgaggagaaggttcaagtattggaggccgaagagagacagtcgggcagtctgaagtgggatgtgcttgggaggtacatgaagtcagtcaactcctggtgcatggtggtaatggcattcctcgtgttggtgatcacgcagggtgctgccaccactaccgactactggcttagtttctggactaaccaggtggacatacaaactctacccgaaggagaaagtaaaaatcctgaattgaacactcaagttggtctgttaaccacgggacagtacctcattgtgcacggcagtgtagtactagccattataatattgacgcaagtcagaatactttccttcgtagtgatgactatgcgagcttcggaaaatcttcataacaccatttacgagaaattgatagtggctgtaatgagatttttcgataccaatccatcgggtcgtgtcttgaacagattctcaaaagatatgggtgcaatggatgagctgttaccgcgaagtatgttggaaacggttcagatgtacctgtctctagccagtgtccttgtgctgaacgcgatagccttaccatggacgttgatacccaccacagtgttgatgtttatatttgtattcctattgaagtggtacattaacgctgctcaagctgtgaaacgattggaaggaactacaaagagtccagtgtttggaatgatcaactctactatatctggactctccaccataagaagctccaactctcaggaccgacttctaaactcatttgatgatgcacagaatctccatactagcgctttctacacgtttttgggtggttcaaccgcatttggtctgtacctggacaccctctgtttaatctacctcggaattatcatgtccatcttcattcttggtgatttcggtgagctgataccggtcggtagtgtgggtctggccgtcagtcagtccatggtgctcaccatgatgttgcaaatggccgccaagttcactgccgacttcttgggacagatgacagccgtcgagagggtcctggagtacaccaagctacctaccgaagaaaacatggagactggaccgacgacgccaccaaaagactggccaagtgctggagaggtgacgttctcaaacgtgtacctcaagtactcacctgacgacccacctgtactgaaggacctgaactttgcaatcaagagtggatggaaggtcggagtagttggtagaactggtgctggcaagtcatcgttgatatcagctctgttccggctcagcgacattacaggcagcatcaaaattgatggcctcgatactcaaggaattgccaagaagcttttgagatcaaaaatatcaataattccgcaagagccagtgttgttctctgcctcgctgcgttacaatctggacccattcgacaactacaacgatgaagatatttggagagcattggaacaggtggaactaaaggagagtataccagccctcgattacaaagtgtcggaaggcggcaccaacttctcgatgggccaacgtcagctggtatgcctagcgcgcgccatcctccgctcaaataaaattctcatcatggacgaagctaccgctaacgtcgatcctcagacggatgctttgatccagaaaacaattcgtaaacaatttgcaacgtgcacggtgctcacgatcgcgcatcgactgaacaccattatggattcagatcgagtactagtcatggaccagggagtggccgcggagttcgaccacccctacatcttgctatctaaccccaatagcaagttctcttcaatggtgaaggaaacaggcgacaacatgtcccggatcttgttcgaagtagccaaaacaaaatatgaaagtgattccaaaactgctgactacaaagacgatgacgacaagtag

SEQ ID NO: 13 - protein, 1367 aa, synthetic construct
  VARIANT 321: note = X can be any amino acid
  VARIANT 351: note = X can be any amino acid
MDTKKGSKNDSKSKPPPPKPKSVPNVFKRTLFCWMLPIFYFGNRRDLEESDLPPPKNMYQSKMLGDKLERSWLKEEHEAKLAGRKPKFTKVLFKTFIWSYIPGGLMQATSIGLRTASPLLFSQLLRYWAADSPVDRETAMYYAISMILANWASSFMNHHGVLFCQQFGMKLRCAVGSLMFRKIMRMSNGSLGDTAAGKVVNILSNDLQRFDLSMVFLHYVWIIPLQIAAVIYLGYLQAGTAAFIGFAALIIIAINNFPGGLGQYLGKIRLRTAEKTDNRIKIMSEVINGIQVIKMYAWEIPFQKVVGQKRAEELKEVKIAXLRTVFLGFMMFTERAALFFTVLTYVLLGNXMSANVFYPLQQFMSAAQVNITLILPMVLSFTAELFVSLGRVEKFLLLEDRPDLKGHPEETSSNLFRNMSADGFPETNGSIRPLSYHRKSEASPIEPGSEIQLKVPKFARSVSYQEDSALVLHDVSASWTGDPNMMALKNISMRLRKGKLCAIIGAVGSGKSSILQLLLKELPSATGTISIYGKMSYACQEAWLFPSTVRENILFGLPFEPGKYKKVCRVCALEKDFKQFPYGDQTLVGERGVSLSGGQRARINLARAVYREADIYLLDDPLSAVDANVGRQLFEGCINGYLRGRTRILVTHQIHFLKAADYIVVLNEGRIENMGTFDELVSSGKEFSMMLSQLQEGKEKDTESVGSRSIEEKEKPTLKTMISVNEGEEVQEFEAQKMKEEERQSGNLRWEVISAYFRSGGHVCFILFALLVVVLATTCAASVDYWVSYWTNQMAAYEESLGGESIEPGLDVQAGRFTVGQYLIIHGCLVAALVLMVNLRVFPFAYLCVSASAKLHNQMFSTMLRGVMRFFDTSSSGRILNRFTKDMGSLDEILPRTLLDVLQIYGTLTAILVLNAIALYWTLVPSAVLLVLFGFMVRIYLKAAQGIKRLEGTTKSPMFGTVTSSLSGISTIRASNAQERLIEQFDINQVSHTTSWNSYLNGGTTFGFYLDTMCLVYMTTIIFVFLFIDFGDAIPVGSVGLAVTQSNTLTLMLQHGARMLVEFLAQLTSVERILEYTRIDTEPDLFQGKVEMPPNWPYQGRIEFQNVSLRYAPNEQPVLKNLNIVIESGNKIGIVGRTGAGKSSLISALFRFAYLDGLISIDGLDTSLISRQGLRSKISIIPQEPILFSATIRYNLDPFDIYSDDDLWRALEQVDLKSAVPSLDFKVTEGGSNFSVGQRQLMCLARAVLRSNQILIMDEATANVDPQTDNFIQETIRRQFVSCTVLTIAHRLNTIMDSDKVLVMSSGQVAEYDHPYVLLSDPNSHFSAMVRETGEKNSANLFQVAKDAYFQSNLKENARDYKDDDDK

SEQ ID NO: 14 - DNA, 4104 bp, synthetic construct
  source 1..4104: mol_type = other DNA
atggatacaaagaaaggaagtaaaaatgactcgaaatccaaaccgccgccgcctaaaccaaagagtgtgccaaacgtgtttaagaggacattgttctgctggatgctgcctatattttactttggcaaccgcagagacctggaggaaagtgatcttccacctcctaagaatatgtatcaatccaagatgctcggagataaattggaaaggagttggctgaaagaagaacatgaagcgaaactagccggacgaaagccgaaatttacgaaggttttattcaaaacattcatttggtcttatataccaggcggtttgatgcaggctacctcaattggcctacgtacggcatctccgctgctgttcagccaactgctccgatattgggcagctgactccccggtggaccgggagacggccatgtactacgcgatcagtatgatcctcgccaactgggcgtcatccttcatgaaccaccacggagtactgttctgtcagcagtttggtatgaagctgaggtgtgccgtcggatctttaatgttcagaaagataatgcgtatgagcaacggctctctaggtgacactgcagcgggtaaggtagtgaacatattatcgaacgacctgcagaggttcgacttgtcaatggtgttcctccactacgtatggattattccgctacaaattgccgcagtcatatatctgggctacctacaagctggaaccgcggctttcattggattcgctgccttaatcatcatagccataaataattttccaggtggtttgggccagtacctaggtaaaattcgacttcgcaccgctgaaaaaacagacaacagaattaaaatcatgagcgaagttataaacggaatacaggtgattaaaatgtacgcatgggagattcctttccaaaaagtagtcggccagaaacgtgcggaagagttgaaggaagtgaagattgctnntctgaggaccgtgttcttaggatttatgatgttcaccgaaagagctgcgcttttcttcaccgtactcacttatgtcttgttggggaacngtatgtctgctaatgtgttctacccgctgcagcaatttatgagcgccgctcaagtaaacattacattaatcttgcctatggtgctatcgttcactgctgagctcttcgtgtcccttggaagagtcgagaagttcctgttactagaggacaggccagacttgaaggggcatccggaagaaacatcgtccaatttattccgcaacatgtcggccgatggcttcccggagaccaacggctctatccgaccgctctcctaccatcggaaatctgaagcatcaccaatagagccgggtagtgagatacagctgaaggttcctaagtttgcacggagtgtgtcgtatcaggaggactcagcgctagtgttacacgatgtgtcagccagttggaccggggaccccaacatgatggcgctgaagaacatctccatgcgactgcggaagggaaagctgtgcgctatcattggtgcagtcggttctgggaagtcctcgattttgcaactactactaaaagaattgccttcggcgactggcactatttcaatatatggcaagatgtcgtacgcttgccaagaggcgtggctgttccccagcactgtccgggagaatatacttttcggtttgccgtttgaacccggaaaatataagaaggtgtgccgagtgtgtgcactcgagaaggacttcaagcagttcccgtacggggaccagacgttggtgggggagaggggggtgtccctctccggaggccaacgtgcccgaatcaatttggcgcgcgctgtttacagagaggccgatatttaccttctcgatgatccgctgtcggctgtggacgctaacgttggacgtcagctatttgagggatgtatcaacggttacctgcgtgggcgcactaggatcctggtcacacatcagattcacttcttgaaagctgctgattacatcgttgtactaaacgagggccgtattgaaaatatgggaacatttgacgaattggtttcgtccggcaaagaattctcaatgatgctctctcagctacaggaaggcaaagaaaaggacacggagagtgttgggtcacggagtatcgaagaaaaagaaaaaccaactctaaagaccatgatcagtgtgaatgaaggtgaggaggtacaagagtttgaagcacagaagatgaaggaagaagagagacaatctggcaacctccgctgggaagtgatctctgcgtacttccgatcgggagggcacgtctgtttcatcttatttgcactcttggtcgttgtactggccactacctgcgccgcttctgttgactactgggtcagttattggaccaatcaaatggctgcatatgaggagtccttaggcggagaaagtatcgaacctggcttggacgtgcaggcgggtcggttcacagtgggacagtatctgatcatccacggctgcctggtggctgccttggtgttgatggtcaacctgcgagtcttccccttcgcctacctctgtgtatccgcctcagccaagctacataaccagatgttctcgactatgctgagaggagtcatgaggttcttcgacaccagttcctcaggtcgtatcttgaatcgttttaccaaagacatgggttctctcgatgagatcctgcctcgtaccttactggatgtactacagatctacggaacattgactgctatcctggtactcaacgccattgctctatactggaccctggtaccttcagcagttctcctagtgttattcggctttatggtacgaatatacttgaaggcagctcaaggcattaaacgattggaaggcacaacaaagagtccaatgtttggaactgtgacgtcatcattgagtggtatttccactatcagggcatccaatgctcaagagagattgatagaacagtttgacatcaaccaggtaagtcatactacttcgtggaacagttacttgaatggaggaacgacgtttggtttctatttagatacaatgtgcctggtatacatgactactatcatatttgtgttcctgttcatcgatttcggggacgcgatcccagtgggcagcgtagggctagcggtgacgcagagcaacacactgacgctgatgctgcagcacggcgcgcggatgttggtcgagttcctcgcgcagctcaccagcgtcgagcgcatcctcgagtacacgcgcatcgacaccgagccagacttgttccaaggaaaggttgaaatgccgccaaattggccctaccaaggaaggatcgagtttcaaaacgtttctctacggtacgctccaaatgaacaacccgtccttaaaaatttgaacattgttatcgaaagcggaaacaagatcgggatcgtggggcggacgggcgccggcaaatcttctttgatatcagcattattccgattcgcctacctcgacggattaatatccatagatggactcgatacttcgctgatttcgagacagggactgcgatcgaaaatttcaattattccgcaagaaccgatcctgttctctgcgacaatcagatacaatttggatccgtttgacatttacagcgacgacgacctctggagagccctcgaacaggtcgacttgaagtctgctgtaccgtccttggacttcaaagtaacagaaggaggttctaatttctcagtgggccagcggcagctgatgtgtttggcgcgcgctgtgttgagatccaatcagatcttaattatggacgaagctacggcgaacgtagatcctcagacggataacttcattcaggagacgattcgccggcagttcgtgtcgtgcaccgtgctgacgatagctcacaggttgaacacgatcatggactccgacaaggtgctcgtgatgagcagcggccaggtggcggagtacgaccacccgtacgtgctgctctccgaccctaacagtcacttctccgccatggtgcgggagaccggcgagaagaacagtgctaacttattccaagtagccaaagacgcttacttccaaagtaatctgaaagaaaacgcgagagactacaaagacgatgacgacaagtag

SEQ ID NO: 15 - protein, 66 aa, synthetic construct
MGYYKKYKEEYYTVKKTYYKKYYEYDKKDYDCDYDKKYDDYDKKYYDHDKKDYDYVVEYKKHKKHY

SEQ ID NO: 16 - DNA, 376 bp, synthetic construct
  source 1..376: mol_type = other DNA
ggataaatcgtttgggccgatgaaaaatcggctctttattttgatttgtttttgtgtcatctgtctttttctatcatttggacagcccttttttccttctatgattttaactgtccaagccgcaaaatctactcgccgtataataaagcgtagtaaaaataaaggaggagtatatatgggttattacaaaaaatacaaagaagagtattatacggtcaaaaaaacgtattataagaagtattacgaatatgataaaaaagattatgactgtgattacgacaaaaaatatgatgactatgataaaaaatattatgatcacgataaaaaagactatgattatgttgtagagtataaaaagcataaaaaacactactaa

SEQ ID NO: 17 - protein, 195 aa, synthetic construct
MGHYSHSDIEEAVKSAKKEGLKDYLYQEPHGKKRSHKKSHRTHKKSRSHKKSYCSHKKSRSHKKSFCSHKKSRSHKKSYCSHKKSRSHKKSYRSHKKSRSYKKSYRSYKKSRSYKKSCRSYKKSRSYKKSYCSHKKKSRSYKKSCRTHKKSYRSHKKYYKKPHHHCDDYKRHDDYDSKKEYWKDGNCWVVKKKYK

SEQ ID NO: 18 - DNA, 748 bp, synthetic construct
  source 1..748: mol_type = other DNA
aaattggattgattcttcatccataatcctccttacaaattttaggcttttatttttataagatctcagcggaacacttatacactttttaaaaccgcgcgtactatgagggtagtaaggatcttcatccttaacatatttttaaaaggaggatttcaaaatgggccactattcccattctgacatcgaagaagcggtgaaatccgcaaaaaaagaaggtttaaaggattatttataccaagagcctcatggaaaaaaacgcagtcataaaaagtcgcaccgcactcacaaaaaatctcgcagccataaaaaatcatactgctctcacaaaaaatctcgcagtcacaaaaaatcattctgttctcacaaaaaatctcgcagccacaaaaaatcatactgctctcacaagaaatctcgcagccacaaaaaatcgtaccgttctcacaaaaaatctcgcagctataaaaaatcttaccgttcttacaaaaaatctcgtagctataaaaaatcttgccgttcttacaaaaaatctcgcagctacaaaaagtcttactgttctcacaagaaaaaatctcgcagctataagaagtcatgccgcacacacaaaaaatcttatcgttcccataagaaatactacaaaaaaccgcaccaccactgcgacgactacaaaagacacgatgattatgacagcaaaaaagaatactggaaagacggcaattgctgggtagtcaaaaagaaatacaaataa

SEQ ID NO: 19 - RNA, 379 nt, synthetic construct
  source 1..379: mol_type = other RNA
tctagagtcctcatgcaaactcaggtttaaatatcgttttcaaatcaattgtccaagagcagcattacaaatagataagtaatttgttgtaatgaaaaacggacatcacctccattgaaacggagtgatgtccgttttactatgttattttctagtaatacatatgtatagagcaacttaatcaagcagagatattttcacctatcgatgaaaatatctctgctttttctttttttatttggtatatgctttacttgtaatcgaaaataaagcactaatagggtgtttttgcccatcccctcgaaaagggggaataagaaaaaataggatgtttttgtagatggagcgccagaggacctgcaggcatgcaagctctaga

SEQ ID NO: 20 - protein, 28 aa, synthetic construct
MDNNPNINECIPYNCLSNPEVEVLGGER

SEQ ID NO: 21 - protein, 27 aa, synthetic construct
MENNIQNQCVPYNCLNNPEVEILNEER

SEQ ID NO: 22 - DNA, 5968 bp, synthetic construct
  source 1..5968: mol_type = other DNA
gcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccaaaccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattagcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtacggcgcgccttaaggaacgtacagacggcttaaaagcctttaaaaacgtttttaaggggtttgtagacaaggtaaaggataaaacagcacaattccaagaaaaacacgatttagaacctaaaaagaacgaatttgaactaactcataaccgagaggtaaaaaaagaacgaagtcgagatcagggaatgagtttataaaataaaaaaagcacctgaaaaggtgtctttttttgatggttttgaacttgttctttcttatcttgatacatatagaaataacgtcatttttattttagttgctgaaaggtgcgttgaagtgttggtatgtatgtgttttaaagtattgaaaacccttaaaattggttgcacagaaaaaccccatctgttaaagttataagtgaccaaacaaataactaaatagatgggggtttcttttaatattatgtgtcctaatagtagcatttattcagatgaaaaatcaagggttttagtggacaagacaaaaagtggaaaagtgagaccatggagagaaaagaaaatcgctaatgttggttactttgaacttctgcatattcttgaatttaaaaaggctgaaagagtaaaagattgtgctgaaatattagagtataaacaaaatcgtgaaacaggcgaaagaaagttgtatcgagtgtggttttgtaaatccaggctttgtccaatgtgcaactggaggagagcaatgaaacatggcattcagtcacaaaaggttgttgctgaagttattaaacaaaagccaacagttcgttggttgtttctcacattaacagttaaaaatgtttatgatggcgaagaattaaataagagtttgtcagatatggctcaaggatttcgccgaatgatgcaatataaaaaaattaataaaaatcttgttggttttatgcgtgcaacggaagtgacaataaataataaagataattcttataatcagcacatgcatgtattggtatgtgtggaaccaacttattttaagaatacagaaaactacgtgaatcaaaaacaatggattcaattttggaaaaaggcaatgaaattagactatgatccaaatgtaaaagttcaaatgattcgaccgaaaaataaatataaatcggatatacaatcggcaattgacgaaactgcaaaatatcctgtaaaggatacggattttatgaccgatgatgaagaaaagaatttgaaacgtttgtctgatttggaggaaggtttacaccgtaaaaggttaatctcctatggtggtttgttaaaagaaatacataaaaaattaaaccttgatgacacagaagaaggcgatttgattcatacagatgatgacgaaaaagccgatgaagatggattttctattattgcaatgtggaattgggaacggaaaaattattttattaaagagtagttcaacaaacgggccatattgttgtataagtgatgaaatactgaatttaaaacttagtttatatgtggtaaaatgttttaatcaagtttaggaggaattaattatgaagtgtaatgaatgtaacagggttcaattaaaagagggaagcgtatcattaaccctataaactacgtctgccctcattattggagggtgaaatgtgaatacatcctattcacaatcgaatttacgacacaaccaaattttaatttggctttgcattttatctttttttagcgtattaaatgaaatggttttgaacgtctcattacctgatattgcaaatgattttaataaaccacctgcgagtacaaactgggtgaacacagcctttatgttaaccttttccattggaacagctgtatatggaaagctatctgatcaattaggcatcaaaaggttactcctatttggaattataataaattgtttcgggtcggtaattgggtttgttggccattctttcttttccttacttattatggctcgttttattcaaggggctggtgcagctgcatttccagcactcgtaatggttgtagttgcgcgctatattccaaaggaaaataggggtaaagcatttggtcttattggatcgatagtagccatgggagaaggagtcggtccagcgattggtggaatgatagcccattatattcattggtcctatcttctactcattcctatgataacaattatcactgttccgtttcttatgaaattattaaagaaagaagtaaggataaaaggtcattttgatatcaaaggaattatactaatgtctgtaggcattgtattttttatgttgtttacaacatcatatagcatttcttttcttatcgttagcgtgctgtcattcctgatatttgtaaaacatatcaggaaagtaacagatccttttgttgatcccggattagggaaaaatataccttttatgattggagttctttgtgggggaattatatttggaacagtagcagggtttgtctctatggttccttatatgatgaaagatgttcaccagctaagtactgccgaaatcggaagtgtaattattttccctggaacaatgagtgtcattattttcggctacattggtgggatacttgttgatagaagaggtcctttatacgtgttaaacatcggagttacatttctttctgttagctttttaactgcttcctttcttttaggaacaacatcatggttcatgacaattataatcgtatttgttttaggtggcctttcgttcaccaaaacagttatatcaacaattgtttcaagtagcttgaaacagcaggaagctggtgctggaatgagtttgcttaactttaccagctttttatcagagggaacaggtattgcaattgtaggtggtttattatccatacccttacttgatcaaaggttgttacctatggaagttgatcagtcaacttatctgtatagtaatttgttattacttttttcaggaatcattgtcattagttggctggttaccttgaatgtatataaacattctcaaagggatttctaaatcgttaagggatcaactttgggagagagttcaaaattgatcctttttttataacaggaatttaaccgtaccgggccccccctcgag

SEQ ID NO: 23 - DNA, 4870 bp, synthetic construct
  source 1..4870: mol_type = other DNA
aagcttctagagatctgcaggtcgacggatccccgggaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcagggggcggagcctatggaaaaacgctttgccc

SEQ ID NO: 24 - protein, 8 aa, synthetic construct
DYKDDDDK

SEQ ID NO: 25 - DNA, 24 bp, synthetic construct
  source 1..24: mol_type = other DNA
gactacaaagacgatgacgacaag

SEQ ID NO: 26 - protein, 10 aa, synthetic construct
EQKLISEEDL

SEQ ID NO: 27 - protein, 8 aa, synthetic construct
HHHHHHHH

SEQ ID NO: 28 - DNA, 25 bp, synthetic construct
  source 1..25: mol_type = other DNA
gtatctcgagaaatcgtttgggccg

SEQ ID NO: 29 - DNA, 25 bp, synthetic construct
  source 1..25: mol_type = other DNA
gtatctcgagggatgaacaaacagc

SEQ ID NO: 30 - DNA, 30 bp, synthetic construct
  source 1..30: mol_type = other DNA
tgagccgcctcctccgtagtgttttttatg

SEQ ID NO: 31 - DNA, 30 bp, synthetic construct
  source 1..30: mol_type = other DNA
tgagccgcctcctcctttgtatttcttttt

SEQ ID NO: 32 - DNA, 31 bp, synthetic construct
  source 1..31: mol_type = other DNA
cataccatggtgatgctgagccgcctcctcc

SEQ ID NO: 33 - DNA, 46 bp, synthetic construct
  source 1..46: mol_type = other DNA
cataccatggtgatgctgatcctcctcctcctgagccgcctcctcc

SEQ ID NO: 34 - DNA, 25 bp, synthetic construct
  source 1..25: mol_type = other DNA
gtatccatggatggataacaatccg

SEQ ID NO: 35 - DNA, 26 bp, synthetic construct
  source 1..26: mol_type = other DNA
cataaggatccttacttttgtgctct

SEQ ID NO: 36 - DNA, 38 bp, synthetic construct
  source 1..38: mol_type = other DNA
ctagactcgagagtctgacagctcgaggtcaagcgtag

SEQ ID NO: 37 - DNA, 38 bp, synthetic construct
  source 1..38: mol_type = other DNA
gatcctacgcttgacctcgagctgtcagactctcgagt

SEQ ID NO: 38 - protein, 5 aa, synthetic construct
GGGGS

SEQ ID NO: 39 - protein, 74 aa, synthetic construct
AGSGGSGGSGGSPVPSTPPTPSPSTPPTPSPSPVPSTPPTNSSSTPPTPSPSPVPSTPPTPSPSTPPTPSPSAS

SEQ ID NO: 40 - protein, 66 aa, synthetic construct
AGSGGSGGSGGSPVPSTPPTPSPSTPPTPSPSGGSGNSSGSGGSPVPSTPPTPSPSTPPTPSPSAS

SEQ ID NO: 41 - protein, 74 aa, synthetic construct
AGSGGSGGSGGSPVPSTPPTPSPSTPPTPSPSPVPSTPPTPSPSTPPTPSPSPVPSTPPTPSPSTPPTPSPSAS

SEQ ID NO: 42 - protein, 152 aa, synthetic construct
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPSAS

SEQ ID NO: 43 - protein, 144 aa, synthetic construct
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSAS

SEQ ID NO: 44 - protein, 242 aa, synthetic construct
AGPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGAS

SEQ ID NO: 45 - protein, 262 aa, synthetic construct
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPSAS

SEQ ID NO: 46 - protein, 121 aa, synthetic construct
AGSGGSGGSGGSPVPSTPPTPSPSTPPTPSPSQIFVKTLTGKTITLEVEPSDTIENVKAKIQDKEGIPPDQQRLIFAGKQLEDGRTLSDYNIQKESTLHLVLRLRGGGGSGGSGGSGGSAS

SEQ ID NO: 47 - protein, 326 aa, synthetic construct
AGSGGSGGSGGSPVPSTPPTPSPSTPPTPSPSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSAS

SEQ ID NO: 48 - protein, 606 aa, synthetic construct
AGSGGSGGSGGSGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSPSAS

SEQ ID NO: 49 - protein, 46 aa, synthetic construct
AGSGNSSGSGGSGGSGNSSGSGGSPVPSTPPTPSPSTPPTPSPSAS

SEQ ID NO: 50 - protein, 141 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 51 - protein, 245 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGGAS

SEQ ID NO: 52 - protein, 349 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGGAS

SEQ ID NO: 53 - protein, 451 aa, synthetic construct
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAGGGGSGGGGSGGGGAS

SEQ ID NO: 54 - protein, 7 aa, synthetic construct
GGGGSAS

SEQ ID NO: 55 - protein, 12 aa, synthetic construct
GGGGSGGGGSAS

SEQ ID NO: 56 - protein, 17 aa, synthetic construct
GGGGSGGGGSGGGGSAS

SEQ ID NO: 57 - protein, 27 aa, synthetic construct
GGGGSGGGGSGGGGSGGGGSGGGGSAS

SEQ ID NO: 58 - protein, 32 aa, synthetic construct
GGGGSGGGGSGGGGSGGGGSGGGGSGGGGSAS

SEQ ID NO: 59 - protein, 37 aa, synthetic construct
AGGGSGGGGSGGGGSGGGGSGGGGSGGGGSGGGGSAS

SEQ ID NO: 60 - protein, 42 aa, synthetic construct
AGGGSGGGGSGGGGSGGGGSGGGGSGGGGSGGGGSGGGGSAS

SEQ ID NO: 61 - protein, 47 aa, synthetic construct
AGGGSGGGGSGGGGSGGGGSGGGGSGGGGSGGGGSGGGGSGGGGSAS

SEQ ID NO: 62 - protein, 74 aa, synthetic construct
AGSGGSGGSGGSPVPSTPPTNSSSTPPTPSPSPVPSTPPTNSSSTPPTPSPSPVPSTPPTNSSSTPPTPSPSAS

(19) United States
(12) Patent Application Publication
(10) Pub. No.: US 2025/0059241 A1
(43) Pub. Date: Feb. 20, 2025

(54) COMPOSITIONS AND METHODS FOR SCREENING INSECTICIDAL PROTEINS

(71) Applicants: SYNGENTA CROP PROTECTION AG, BASEL (CH); HOKKAIDO UNIVERSITY, Sapporo (JP)

(72) Inventors: Sachiko KASHOJIYA, Sapporo (JP); Shin-ichiro ASANO, Sapporo (JP); Takashi YAMAMOTO, Dublin, CA (US)

(21) Appl. No.: 18/728,805

(22) PCT Filed: Jan. 13, 2023
(86) PCT No.: PCT/US2023/060676

§ 371 Date: Jul. 12, 2024

Related U.S. Application Data
(60) Provisional application No. 63/299,567, filed on Jan. 14, 2022.

(51) Int. Cl. C07K 14/325; A01N 63/23; A01P 7/04; G01N 33/50

(52) U.S. Cl. CPC C07K 14/325; A01N 63/23; A01P 7/04; G01N 33/5085; C07K 2319/74

(57) ABSTRACT

Provided herein are methods for screening for and identifying insecticidal proteins along with recombinant nucleic acids, DNA constructs, vectors, fusion proteins, and spores useful in the provided methods. The provided methods enable effective, high throughput screening of candidate insecticidal proteins, including naturally occurring and engineered synthetic proteins.

### CROSS-REFERENCE TO RELATED APPLICATION

The present application claims the benefit of priority to U.S. Provisional Application No. 63/299,567, filed Jan. 14, 2022, the entire contents of which are incorporated by reference for all purposes.

### FIELD

This disclosure relates to the field of plant biotechnology. In particular, it relates to methods for screening insecticidal proteins for desired traits.

### REFERENCE TO A SEQUENCE LISTING SUBMITTED AS AN XML FILE VIA EFS-WEB

This application is accompanied by a sequence listing entitled 109098-1360060.xml, created Jan. 13, 2023, which is approximately 1,03,846 bytes in size. This sequence listing is incorporated herein by reference in its entirety. This sequence listing is submitted herewith via EFS-Web, and is in compliance with 37 C.F.R. § 1.824(a)(2)-(6) and (b).

### BACKGROUND

Insecticidal proteins can include naturally occurring and synthetic proteins. Candidate insecticidal proteins, especially those of Bacillus thuringiensis (Bt), can be engineered (i.e., modified artificially) by DNA shuffling, block swapping, site-directed, saturation, and random mutageneses. These engineering techniques often produce a large number of variants, called a library as a collective group. Such a library may be screened for a desired trait or desired traits. This process requires a high throughput protein production and screening of diversified proteins. Such high throughput screening is time-consuming and expensive. This process typically involves 1) producing a library of diversified insecticidal proteins (i.e., naturally occurring and/or engineered proteins) using protein engineering techniques; 2) cloning the library genes in a host organism such as Escherichia coli or Bacillus species for protein production; 3) individually picking transformants of the host organism containing the library in multi-well plates to produce seed cultures; 4) expressing the library proteins in multi-well plates and isolating them individually; 5) analyzing the isolated proteins for purity and concentration and normalizing the proteins (i.e., by adjusting protein concentrations) for use; 6) assaying the insecticidal activity and other traits of the isolated and analyzed proteins in live insects to identify proteins with desired traits; and 7) repeating the above steps to identify additional proteins with desired traits.

A drawback of current screening processes is that experimental throughput is limited to thousands of samples at most without significant investments in manpower and infrastructure. The rate limiting factors are that the process involves using live insect bioassays and the amounts of isolated protein necessary for the bioassays. As such, increasing throughput over traditional methods described above is highly desirable in the pursuit of finding new receptor specificities.

### BRIEF SUMMARY

This summary is a high-level overview of various aspects of the present disclosure and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. Some of the exemplary embodiments of the present disclosure are discussed below.

The present disclosure is based in part on the discovery by the inventors of a novel method of screening a library of putative toxin proteins and/or protein variants using a cell (particle) sorting technology to identify insect-active toxin proteins. In some embodiments, as described further below, insect-active toxins are tethered individually on carrier particles (e.g., spores) which contain genetic information of the tethered proteins (i.e., tagging).

In one aspect, provided herein is a recombinant nucleic acid encoding a fusion protein. In some embodiments, the recombinant nucleic acid comprises a) a first nucleic acid sequence encoding a spore outer coat polypeptide; b) a second nucleic acid sequence encoding a linker; and c) a third nucleic acid sequence encoding a putative insecticidal polypeptide.

In some embodiments of the recombinant nucleic acids provided herein, the spore outer coat polypeptide is from a Bacillus bacteria. In some embodiments, the spore outer coat polypeptide is CotC, CotG, CotB, CotA, CotD, CotE, CotX, CotY, or CotZ. In some embodiments, the spore outer coat polypeptide is CotC or CotG.

In some embodiments of the recombinant nucleic acids provided herein, the putative insecticidal polypeptide is a Bacillus thuringiensis crystal (Bt Cry) protein. In some embodiments, the Bt Cry protein comprises at least one modification relative to the wild type protein. In some embodiments, the modification is a result of DNA shuffling, block swapping, site-directed mutagenesis, saturation mutagenesis, random mutagenesis, or a combination thereof. In some embodiments, the Bt Cry protein modification results in resistance to serine protease digestion.

In some embodiments, the putative insecticidal polypeptide interacts with a cell surface protein of an insect digestive system epithelial cell. In some embodiments, the cell surface protein is an ATP-Binding Cassette (ABC) transporter protein, a cadherin protein, an aminopeptidase N protein, or an alkaline phosphatase protein. In some embodiments, the cell surface protein is an ABC transporter protein. In some embodiments, the ABC transporter protein is PxABCC2, Bm-ABCC2, Sf-ABCC2, Sf-ABCC3, Dv-ABCB1, Bm-ABCB1, Sf-ABCB1, Bm-ABCA2, or Tc-ABCC4. In some embodiments, the ABC transporter protein is PxABCC2, Bm-ABCC2, Sf-ABCC2, or Sf-ABCC3.

In some embodiments of the recombinant nucleic acids provided herein, the spore outer coat polypeptide and the putative insecticidal polypeptide are structurally isolated by the linker. In some embodiments, the linker comprises at least one amino acid. In some embodiments, the linker is a polypeptide comprising at least two amino acids. In some embodiments, the linker is structurally flexible. In some embodiments, the linker is resistant to protease digestion. In some embodiments, the linker is a polypeptide comprising an amino acid sequence having at least 80% identity to any of SEQ ID NOs: 20, 21, or 38-61.

Also provided are DNA constructs comprising any of the recombinant nucleic acids described herein. Also provided are vectors comprising any of the recombinant nucleic acids or DNA constructs described herein.

Also provided herein are fusion proteins encoded by any of the recombinant nucleic acids, DNA constructs, or vectors described herein.

Also provided herein are spores comprising any of the recombinant nucleic acids, DNA construct, vectors, or fusion proteins described herein. In some embodiments, the spore comprises a fusion protein, and the fusion protein is displayed on the surface of the spore. In some embodiments, the spore is a bacterial spore or a fungal spore.

Also provided herein are compositions comprising a plurality of any of the spores described herein. In some embodiments, each spore in the plurality comprises a recombinant nucleic acid encoding a different putative insecticidal polypeptide.

In another aspect, provided herein are methods of identifying an insecticidal protein. In some embodiments, the methods comprise a) contacting any of the fusion proteins or spores described herein with an insecticidal protein receptor and b) detecting an interaction between the fusion protein or spore and the insecticidal protein receptor. In some embodiments, said interaction indicates that the putative insecticidal protein of the fusion protein or spore is an insecticidal protein. In some embodiments, the insecticidal protein receptor is a purified insecticidal protein receptor protein. In some embodiments, the insecticidal protein receptor is part of an SMA lipid particle. In some embodiments, the insecticidal protein receptor is expressed on a cell. In some embodiments, the cell is an insect cell. In some embodiments, the insecticidal protein receptor is an ABC transporter protein.

In some embodiments of the methods provided herein, detecting the interaction between the fusion protein or spore and the insecticidal protein receptor comprises performing immuno-magnetic separation, flow cytometry, cytotoxicity assays, sequencing, or a combination thereof. In some embodiments, detecting the interaction between the fusion protein or spore and the insecticidal protein receptor protein further comprises isolating the recombinant nucleic acid encoding the fusion protein that interacts with the insecticidal protein receptor protein and performing sequencing to determine the identity of the the insecticidal protein.

### BRIEF DESCRIPTION OF THE DRAWINGS

The present disclosure includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIG. 1 shows a method of amplifying cotC or cotG (Bacillus subtilis outer coat protein gene) by PCR with their own promoters, according to aspects of this disclosure. This method results in connection of a cot gene to a Bacillus thuringiensis cry gene via a linker to form a Cot-Cry fusion protein with a structurally flexible linker.

FIG. 2 shows an example method for constructing spore display vectors with Bacillus subtilis cotC or cotG gene and Bacillus thuringiensis cry1Aa gene, according to aspects of this disclosure. Two Bacillus-E. coli shuttle vectors, pSB634, a fusion of pBC16.1 of Bacillus cereus and pBluescript KS of E. coli and pHY300PLK, a fusion of pAMα1 of Streptococcus faecalis and pACYC177 of E. coli were used.

FIG. 3 shows that Bacillus subtilis cotC or cotG gene was fused with Bacillus thuringiensis cry1Aa or cry1Fa gene with a linker and terminator taken from Bacillus thuringiensis cry1Ac gene, according to aspects of this disclosure. The Arg 27/28 (R27/28) amino acid residues of Cry1 proteins were mutated to Leu (L) to prevent serine protease digestion.

FIG. 4 shows a method of detecting Bacillus thuringiensis Cry1 protein displayed on a Bacillus subtilis spore with anti-Cry1 antibody (Ab), according to aspects of this disclosure. As depicted in the schematic illustration (top panel), the antibody labelled with Alexa Fluor 488 fluorescent dye leads to spore fluorescence upon binding. The bottom panels are microscopic views of the Spore-Cry1Fa (spore displaying the Cry1Fa protein) which became fluorescent upon binding to anti-Cry1 antibody (bottom right panel).

FIG. 5 shows a feasibility study demonstrating that Bacillus spores displaying a Bacillus thuringiensis Cry protein can be sorted by flow cytometry, according to aspects of this disclosure. Those spores displaying the Cry protein were made fluorescent with a fluorescent anti-Cry antibody (e.g., as shown in FIG. 4). Two consecutive sorting experiments were conducted: the left panel was the first sorting and spores in its boxed area were resorted to obtain the right panel.

FIG. 6 shows microscopic views of a Sf9 cell (a Spodoptera frugiperda cell line) expressing the ABCC2 gene of Spodoptera frugiperda (upper panels) (Sf9::Sf-ABCC2) and another cell not expressing the ABCC2 gene (bottom panel), according to aspects of this disclosure. These Sf9 cells were mixed with Spore-Cry1Fa Bacillus subtilis spores displaying the Cry1Fa protein and allowed to bind to spores. Bound Spore-Cry1Fa spores were made visible as bright spots with Alexa-labelled anti-Cry1Fa antibody by fluorescence microscopy (upper right panel). The host Sf9 cells were also visible by fluorescence microscopy as they expressed a GFP gene. The bottom panels are negative control showing Spore-Cry1Fa did not bind to Sf9 cells expressing no Sf-ABCC2 protein.

FIG. 7 shows microscopic observations of a cytotoxic activity of Spore-Cry1Fa on Sf9 cells expressing Sf-ABCC2, according to aspects of this disclosure. After Spore-Cry1Fa bound to the Sf9 cell, the Cry1Fa protein on the spores caused the cell membrane to disintegrate within an hour (upper panels). Spore-Cry1Fa was visible under the fluorescence microscopy as bright spots with Alexa-labelled anti-Cry antibody bound on the spores. A longer incubation showed the Sf9 cell disintegrated completely (lower panels).

FIG. 8 shows a method of packaging ABC transporter protein in SMALP (Styrene Maleic Acid Lipid Particle), according to aspects of this disclosure. The transporter protein expressed in the insect cell membrane was extracted with SMA (Styrene Maleic Acid). In this figure, “Extraction” refers to removal of the ABC transporter protein from insect cell membrane with SMA and packaging of the transporter protein in SMALP (Styrene Maleic Acid Lipid Particle) with its own phospholipids.

FIG. 9 shows affinity column purification of Sf-ABCC2-FLAG in SMALP using anti-FLAG antibody, according to aspects of this disclosure. ABC transporter proteins used in the Examples herein were labelled with a FLAG tag on the C-terminus. SMALP-Sf-ABCC2-FLAG was loaded on 200 d anti-FLAG-antibody-immobilized agarose gel packed in a 1 ml Pierce™ Spin Column (Thermo-Fisher). The column was washed with TBS containing 10% glycerol (TBS-glycerol), and Sf-ABCC2 was eluted with 1.2 ml elution buffer (0.1 mg/ml FLAG peptide in TBS-10% glycerol). Column eluate was fractionated in six 200 μl fractions and analyzed by dot blotting with Alexa-labelled anti-FLAG antibody. All fractions (“Fr.”) including the final spin off (Fr. 7) except for the first one (Fr. 1) showed strong fluorescent signal confirming Sf-ABCC2 was successfully eluted from the affinity column.

FIG. 10 shows Bacillus subtilis colonies growing on LB-tet-agar plate, according to aspects of this disclosure. These colonies were from Spore-pSB634 (no Cry protein displayed) or Spore-Cry1Fa spores sorted by immuno-magnetic sorting. These spores were allowed to bind to SMALP-Sf-ABCC2-FLAG (e.g., as purified in FIG. 9) and sorted with anti-FLAG-antibody-immobilized magnetic beads which were designed to capture only ABCC2-bound spores. Sorted spores were plated on a LB-tet-agar plate and incubated at 37° C. to germinate and form colonies in 16 hr. Spore-pSB634 sorted under the identical protocol as a negative control showed only a few colonies (left panel) indicating almost no binding between this spore sample and Sf-ABCC2 in SMALP. The sorting of Spore-Cry1Fa produced colonies too many to count (right panel).

FIG. 11 shows sorting of Spore-Cry1Aa (Bacillus subtilis spores displaying Bacillus thuringiensis Cry1Aa protein) by flow cytometry, according to aspects of this disclosure. Spore-Cry1Aa-Bm-ABCC2 complex was made fluorescent by mixing with anti-FLAG-mouse-antibody and Alexa-labeled anti-mouse IgG-antibody sequentially. A treatment with 0.2% SDS (as described in Example 8 herein) was made to lower the population of fluorescent spores for cell cytometry by partially breaking the binding between Cry1Aa and ABC transporter proteins. This is to demonstrate the feasibility of spore separation by flow cytometry. Microscopic observation revealed only a small number of spores retained the fluorescent dye. Those fluorescent spores were sorted by a flow cytometer, On-chip Sort cell sorter (On-Chip Biotechnologies). The cell sorter pulled out about 1000 spores as fluorescent, presumably bound to Bm-ABCC2. The sorted 1000 fluorescent spores (boxed in the cell sorter output) were sampled and plated on a LB-agar plate. The LB plate was copied onto a LB-tet-agar plate. It was found over 800 spores were germinated to form colonies on the tetracycline-selection plate, indicating they contain the plasmid directing Cry1Aa to the spore surface and also conferring tetracycline resistance. This sorting process indicates captured spores can be recovered and the sequence of the Cry protein can be deduced by DNA sequence determination of the plasmid.

### DETAILED DESCRIPTION

### I. Introduction

The present disclosure is based in part on the discovery by the inventors of an effective, high throughput method for screening libraries of putative insecticidal proteins using spore display technology. While spore display technology has previously been utilized to display enzymes and antigen (e.g., for vaccine development), the present disclosure provides the first application of spore display to screening of insecticidal proteins for receptor binding capability.

Provided herein are methods for identifying insecticidal proteins along with recombinant nucleic acids, DNA constructs, vectors, fusion proteins, and spores useful in the provided methods. The provided methods enable effective, high throughput screening of candidate insecticidal proteins, including naturally occurring and engineered synthetic proteins. As detailed herein, embodiments of the provided methods comprise display of functionally active insecticidal proteins (e.g., Bacillus thuringiensis crystal [Bt Cry] proteins) on the surface of microbial spores (e.g., bacterial spores or fungal spores). In some embodiments, the spore surface display comprises use of fusion proteins comprising a spore outer coat protein linked (e.g., by a structurally flexible linker) to a candidate insecticidal protein. In some embodiments, upon expression of the fusion protein in a spore, the fusion protein migrates to the spore surface to display the insecticidal protein positioned toward the spore exterior (e.g., toward the solvent or medium).

Before the experiments described in the Examples herein, it was not known if an insecticidal protein (e.g., Bt Cry toxin) could be displayed functionally (i.e., where the insecticidal proteins receptor binding and subsequent insecticidal functions are preserved) on a spore surface. In some embodiments, the methods provided herein comprise fusion proteins utilizing a structurally flexible linker to anchor a putative insecticidal protein to a spore outer coat protein with a certain degree of freedom or full freedom so that the insecticidal protein (e.g., the Cry protein) moiety can fold properly and so that its receptor binding domain is exposed to the receptor.

In some embodiments of the methods provided herein, the insecticidal protein is kept attached to the spore during screening and the spore, which comprises a gene encoding the insecticidal protein (e.g., in a spore-display vector, as detailed below), carries the sequence tag of the insecticidal protein anchored on its surface. When a spore carrying a desired variant of the insecticidal protein is found during the screening, the sequence of that variant can be determined by sequencing the insecticidal protein gene in the spore. This eliminates the need of separating individual clones in multi-well plates. The spore display system provided herein also has the advantage of allowing flow cytometric sorting of spores, whereas E. coli phages used in phage display systems can not be sorted by flow cytometry.

Recent attempts to use a related technique, phage display, to screen Bt Cry mutants for receptor binding activity have proven unsuccessful (see Fujii et al., 2012, Mol. Biotech. 54:888-899). A limitation to phage display is that, since the phage particles are produced in E. coli, heterogeneous genes from Bacillus species (e.g., Bt Cry proteins and mutant versions thereof) may not be reliably expressed. This is especially problematic when a screening library contains a wide variety of protein sequences. In fact, Fujii et al. (2012, Mol. Biotech. 54:888-899) found that no improvement of the insecticidal activity was obtained by phage display. Additionally, the result indicates a possibility that the structure of phage displayed Bt Cry proteins may not be the same as that of the native protein. The methods and compositions provided herein overcome this limitation. In some embodiments, the methods herein allow homogeneous expression of Bacillus genes (e.g., Bt Cry insecticidal protein genes) in Bacillus hosts.

In summary, the methods provided herein have several advantages over other insecticidal protein screening techniques, including but not limited to: 1) higher throughput screening of candidate insecticidal protein libraries (e.g., using cell sorting technologies such as immune-magnetic separation and flow cytometry); 2) higher tolerance for expression and screening of candidate insecticidal proteins from Bacillus species (e.g., Bt Cry proteins) relative to phage display, as spores can be produced in Bacillus species; and 3) increased flexibility in receptors for candidate insecticidal proteins.

### II. Terminology

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject.

As used in herein, the singular forms “a”, “an” and “the” include plural referents unless the content clearly dictates otherwise. Thus, for example, reference to “an antibody” optionally includes a combination of two or more such molecules, and the like.

The term “about” as used herein refers to the usual error range for the respective value readily known to the skilled person in this technical field, for example ±20%, ±10%, or ±5%, are within the intended meaning of the recited value.

As used herein, the term “comprising” or “comprise” is open-ended. When used in connection with a subject nucleic acid (or amino acid sequence), it refers to a nucleic acid sequence (or an amino acid sequence) that includes the subject sequence as a part or as its entire sequence.

The term “plurality” refers to more than one entity. Thus, a “plurality of individuals” refers to at least two individuals. In some embodiments, the term plurality refers to more than half of the whole. For example, in some embodiments a “plurality of a population” refers to more than half the members of that population.

The term “next generation discovery technology” or “NGDT” is used to refer to certain embodiments of the present disclosure, as detailed below.

The term “Cry” refers to a member of the Bacillus thuringiensis crystal protein family, members of which may be insecticidal.

The term “Cry1Fa” refers to a Cry protein (or, when italicized [cry1Fa], the gene encoding the protein) showing high activity against Spodoptera frugiperda (Sf, Fall Armyworm) and known to bind to Sf-ABCC2. The amino acid sequence of Cry1Fa is shown in SEQ ID NO:1, and the nucleotide sequence of the protein coding region of cry1Fa is shown in SEQ ID NO:2.

The term “Cry1Aa” refers to a Cry protein (or, when italicized [cry1Aa], the gene encoding the protein) showing high activity against Bombyx mori (Bm, Silkworm) and Plutella xylostella (Px, Diamondback Moth) and known to bind to Bm/Px-ABCC2. The amino acid sequence of Cry1Aa is shown in SEQ ID NO: 3, and the nucleotide sequence of the protein coding region of cry1Aa is shown in SEQ ID NO:4.

The term “ATP-Binding Cassette Transporters” or “ABC Transporters” refers to a large family of transport system proteins represented in all extant phyla. The term “ABCC” refers to ABC transporter subfamily C, type 2 (ABCC2) protein and/or type 3 (ABCC3) protein (or, when italicized [ABCC], the gene encoding the protein). The amino acid sequence of Px-ABCC2 is shown in SEQ ID NO:5, and the nucleotide sequence of the protein coding region of Px-ABCC2 is shown in SEQ ID NO:6. The amino acid sequence of Bm-ABCC2 is shown in SEQ ID NO:7, and the nucleotide sequence of the protein coding region of Bm-ABCC2 is shown in SEQ ID NO:8. The amino acid sequence of Sf-ABCC2 is shown in SEQ ID NO: 9, and the nucleotide sequence of the protein coding region of Sf-ABCC2 is shown in SEQ ID NO: 10. The amino acid sequence of an Sf-ABCC2 mutant (referred to herein as “Sf-ABCC2-mutant”) from a Cry1Fa-resistant Sf strain is shown in SEQ ID NO:11, and the nucleotide sequence of the protein coding region of Sf-ABCC2-mutant is shown in SEQ ID NO:12. The amino acid sequence of Sf-ABCC3 is shown in SEQ ID NO:13, and the nucleotide sequence of the protein coding region of Sf-ABCC3 is shown in SEQ ID NO:14. As there are differences in the protein sequences within the same class of ABC transporters (e.g., ABCC2 proteins of different insect species), ABCCs are generally referred to herein with an insect species abbreviation, for example, Sf (Fall armyworm, Spodoptera frugiperda), Bm (Silkworm, Bombyx mori), Dv (Western corn rootworm (WCR), Diabrotica virgifera virgifera), Tc (Red flour beetle, Tribolium castaneum), etc., to indicate the source.

As used herein, the term “spore” without modification refers to a spore of any spore-producing microbe. Examples of spore-producing microbes include bacterial and fungal species, as detailed herein below. Spores from a particular source may be indicated herein with a genus or species name, for example, B. thuringiensis spores or Bacillus spores in general. In some embodiments, the present disclosure provides spores comprising a protein (e.g., a protein expressed by the spore which may be expressed on the surface of the spore) and/or a vector (e.g., where no protein is expected to be displayed on the surface of the spore). Such spores are indicated as “Spore-[protein name]” (e.g., Spore-Cry1Fa or Spore-Cry1Aa) or “Spore-[vector name]” (e.g., Spore-pSB634 or Spore-pHY300PLK).

The term “CotC” refers to Bacillus subtilis outer spore coat type C protein (or, when italicized [cotC], the gene encoding the protein). The amino acid sequence of CotC is shown in SEQ ID NO:15, and the nucleotide sequence of cotC (comprising its own promoter) is shown in SEQ ID NO: 16.

The term “CotG” refers to Bacillus subtilis outer spore coat type G protein (or, when italicized [cotG], the gene encoding the protein). The amino acid sequence of CotG is shown in SEQ ID NO:17, and the nucleotide sequence of cotG (comprising its own promoter) is shown in SEQ ID NO: 18.

The term “cry1Ac terminator” refers to the translation terminator of the Bt cry1Ac gene having a double hairpin RNA structure, the nucleotide sequence of which is shown in SEQ ID NO:19.

The terms “Cry1Aa leader sequence” and “Cry1Fa leader sequence” refer to short (˜27 amino acids) natural amino acid sequences attached to the N-terminus of the mature Cry1Aa and Cry1Fa proteins which are removed by protease digestion in the insect gut to convert the protoxin to the mature toxin. In some embodiments, the leader sequence serves as a flexible linker between Cot and Cry proteins. The Cry1Aa leader sequence is shown in SEQ ID NO:20, and the Cry1Fa leader sequence is shown in SEQ ID NO:21.

The term “spore-display vector” refers to a plasmid vector in which an insecticidal protein is cloned with a spore outer coat protein gene to express (or display) the protein (e.g., on the surface of a spores or spores). In the present disclosure, two Bacillus-E. coli shuttle vectors, pSB634 (SEQ ID NO: 22), which contains a Bacillus cereus pBC16.1 and E. coli pBluescript-KS, and pHY300PLK (SEQ ID NO: 23), which contains Streptococcus faecalis pAM-alpha and E. coli pACYC177 were used as examples.

The term “tet” refers to the antibiotic compound tetracycline.

The term “LB” refers to Luria broth. In some embodiments, LB comprises 10 g Tryptone, 5 g Yeast Extract, and 10 g NaCl in 1 L water. In the present disclosure, 1/2 LB means 2-fold diluted LB (e.g., diluted with water), and “LB-Agar” refers to solid LB media made with 1.5% agar.

The term “Sf9” refers to a clonal isolate insect cell line of parental cell line Spodoptera frugiperda Sf21 IPLB-Sf21-AE (derived from pupal ovarian tissue).

The terms “nucleic acid” and “polynucleotide” are used interchangeably and as used herein refer to both sense and anti-sense strands of RNA, cDNA, genomic DNA, mitochondrial DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA and/or RNA. In addition, a polynucleotide disclosed herein, e.g., a circular DNA template, a nucleic acid concatemer disclosed herein, can include either or both naturally occurring and modified nucleotides linked together by naturally occurring and/or non-naturally occurring nucleotide linkages. The nucleic acid molecules can be modified chemically or biochemically or can contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Nucleotide sequences are “complementary” when they specifically hybridize in solution (e.g., according to Watson-Crick base pairing rules). The term also includes codon-optimized nucleic acids that encode the same polypeptide sequence. It is also understood that nucleic acids can be unpurified, purified, or attached, for example, to a synthetic material such as a bead or column matrix.

Unless otherwise indicated, a particular nucleic acid sequence also implicitly encompasses conservatively modified variants thereof, alleles, orthologs, SNPs, and complementary sequences as well as the sequence explicitly indicated.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (e.g., BLAST), or by manual alignment and visual inspection.

In particular, algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

“Polypeptide,” “peptide,” and “protein” are used interchangeably herein to refer to a polymer of amino acid residues. As used herein, the terms encompass amino acid chains of any length, including full-length proteins, wherein the amino acid residues are linked by covalent peptide bonds.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-587 (2013); Xie et la. “Adding amino acids to the genetic repertoire,” 9(6): 548-54 (2005)); and all references cited therein. Beta and gamma amino acids are known in the art and are also contemplated herein as unnatural amino acids.

As used herein, a chemically modified amino acid refers to an amino acid whose side chain has been chemically modified. For example, a side chain can be modified to comprise a signaling moiety, such as a fluorophore or a radiolabel. A side chain can also be modified to comprise a new functional group, such as a thiol, carboxylic acid, or amino group. Post-translationally modified amino acids are also included in the definition of chemically modified amino acids.

Also contemplated are conservative amino acid substitutions. By way of example, conservative amino acid substitutions can be made in one or more of the amino acid residues, for example, in one or more lysine residues of any of the polypeptides provided herein. One of skill in the art would know that a conservative substitution is the replacement of one amino acid residue with another that is biologically and/or chemically similar. The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M).

By way of example, when an arginine to serine is mentioned, also contemplated is a conservative substitution for the serine (e.g., threonine). Nonconservative substitutions, for example, substituting a lysine with an asparagine, are also contemplated.

As used herein, the term “primer” refers to an oligonucleotide that is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to anlplify plant nucleic acids (e.g., using the polymerase chain reaction; PCR).

### III. Recombinant Nucleic Acids, Constructs, Vectors, and Spores

In one aspect, provided herein are recombinant nucleic acids encoding a fusion protein, wherein the recombinant nucleic acid comprises: a) a first nucleic acid sequence encoding a spore outer coat polypeptide; b) a second nucleic acid sequence encoding a linker; and c) a third nucleic acid sequence encoding a putative insecticidal polypeptide.

### A. Spore Outer Coat Polypeptide

In some embodiments of the recombinant nucleic acids, the spore outer coat polypeptide is from any microbe that produces spores. In some embodiments, the microbe is an endospore-producing bacterium. In some embodiments, the bacterium is a species of any of the following genuses: Acetonema, Actinomyces, Alkalibacillus, Ammoniphilus, Amphibacillus, Anaerobacter, Anaerospora, Aneurinibacillus, Anoxybacillus, Bacillus, Brevibacillus, Caldanaerobacter, Caloramator, Caminicella, Cerasibacillus, Clostridium, Clostridisalibacter, Cohnella, Coxiella (i.e., Coxiella burnetii), Dendrosporobacter, Desulfotomaculum, Desulfosporomusa, Desulfosporosinus, Desulfovirgula, Desulfunispora, Desulfurispora, Filifactor, Filobacillus, Gelria, Geobacillus, Geosporobacter, Gracilibacillus, Halobacillus, Halonatronum, Heliobacterium, Heliophilum, Laceyella, Lentibacillus, Lysinibacillus, Mahella, Metabacterium, Moorella, Natroniella, Oceanobacillus, Orenia, Ornithinibacillus, Oxalophagus, Oxobacter, Paenibacillus, Paraliobacillus, Pelospora, Pelotomaculum, Piscibacillus, Planmfilum, Pontibacillus, Propionispora, Salinibacillus, Salsuginibacillus, Seinonella, Shimazuella, Sporacetigenium, Sporoanaerobacter, Sporobacter, Sporobacterium, Sporohalobacter, Sporolactobacillus, Sporomusa, Sporosarcina, Sporotalea, Sporotomaculum, Syntrophomonas, Syntrophospora, Tenuibacillus, Tepidibacter, Terribacillus, Thalassobacillus, Thermoacetogenium, Thermoactinomyces, Thermoalkalibacillus, Thermoanaerobacter, Thermoanaeromonas, Thermobacillus, Thermoflavimicrobium, Thermovenabulum, Tuberibacillus, Virgibacillus, or Vulcanobacillus.

In some embodiments, the spore outer coat polypeptide is from a Bacillus species. In some embodiments, the spore outer coat polypeptide is from a Bacillus species (e.g., Bacillus subtilis) spore outer coat (Cot) protein. In some embodiments, the Cot protein is CotC, CotG, CotB, CotA, CotD, CotE, CotX, CotY, or CotZ. In some embodiments, the Cot protein is CotC or CotG.

In some embodiments of the recombinant nucleic acids provided herein, the first nucleic acid sequence encoding a spore outer coat polypeptide comprises a nucleic acid sequence having at least 60% identity (e.g., at least 65% identity, at least 70% identity, at least 75% identity, at least 80% identity, at least 85% identity, at least 90% identity, at least 95% identity, or at least 99% identity) to SEQ ID NO:16 or SEQ ID NO:18. In some embodiments, the first nucleic acid sequence encodes a spore outer coat polypeptide comprising an amino acid sequence having at least 60% identity to SEQ ID NO:15 or SEQ ID NO:17.

In some embodiments of the recombinant nucleic acids provided herein, the spore outer coat polypeptide is from a spore-producing fungus. In some embodiments, the fungus is from any of the following phyla: Zygemycota, Ascomycota, Basidiomycota, or Oomycota.

### B. Putative Insecticidal Polypeptide

The recombinant nucleic acids provided herein can encode a putative insecticidal polypeptide in any of the following structural classes: App, Cry, Cyt, Gpp, Mcf, Mpf, Mpp, Mtx, Pra, Prb, Spp, Tpp, Vip, Vpa, or Vpb. See, e.g., Crickmore, N., et al. “A structure-based nomenclature for Bacillus thuringiensis and other bacteria-derived pesticidal proteins.” Journal of Invertebrate Pathology, 107438 (2020); doi.org/10.1016/j.jip.2020.107438.

In some embodiments, the insecticidal polypeptide is a Bacillus thuringiensis insecticidal protein including but not limited to a Cry protein, a vegetative insecticidal protein (VIP) and insecticidal chimeras of any of the preceding insecticidal proteins. In some embodiments, the putative insecticidal polypeptide is a Bacillus thuringiensis crystal (Bt cry) protein.

Example Cry proteins include, but are not limited to, Cry1Aa, Cry1Ab, Cry1Ac, Cry1Ad, Cry1Ae, Cry1Af, Cry1Ag, Cry1Ah, Cry1Ai, Cry1Aj, Cry1Ba, Cry1Bb, Cry1Bc, Cry1Bd, Cry1Be, Cry1Bf, Cry1Bg, Cry1Bh, Cry1Bi, Cry1Ca, Cry1Cb, Cry1 Da, Cry1Db, Cry1Dc, Cry1Dd, Cry1Ea, Cry1Eb, Cry1Fa, Cry1Fb, Cry1Ga, Cry1Gb, Cry1Gc, Cry1Ha, Cry1Hb, Cry1Hc, Cry1Ia, Cry1Ib, Cry1Ic, Cry1Id, Cry1Ie, Cry1If, Cry1Ig, Cry1Ja, Cry1Jb, Cry1Jc, Cry1Jd, Cry1Ka, Cry1La, Cry1Ma, Cry1Na, Cry1Nb, Cry2Aa, Cry2Ab, Cry2Ac, Cry2Ad, Cry2Ae, Cry2Af, Cry2Ag, Cry2Ah, Cry2Ai, Cry2Aj, Cry2Ak, Cry2A1, Cry2Ba, Cry3Aa, Cry3Ba, Cry3Bb, Cry3Ca, Cry4Aa, Cry4Ba, Cry4Ca, Cry4Cb, Cry4Cc, Cry5Aa, Cry5Ab, Cry5Ac, Cry5Ad, Cry5Ba, Cry5Ca, Cry5 Da, Cry5Ea, Cry6Aa, Cry6Ba, Cry7Aa, Cry7Ab, Cry7Ac, Cry7Ba, Cry7Bb, Cry7Ca, Cry7Cb, Cry7 Da, Cry7Ea, Cry7Fa, Cry7Fb, Cry7Ga, Cry7Gb, Cry7Gc, Cry7Gd, Cry7Ha, Cry7Ia, Cry7Ja, Cry7Ka, Cry7Kb, Cry7La, Cry8Aa, Cry8Ab, Cry8Ac, Cry8Ad, Cry8Ba, Cry8Bb, Cry8Bc, Cry8Ca, Cry8 Da, Cry8Db, Cry8Ea, Cry8Fa, Cry8Ga, Cry8Ha, Cry8Ia, Cry8Ib, Cry8Ja, Cry8Ka, Cry8Kb, Cry8La, Cry8Ma, Cry8Na, Cry8 Pa, Cry8Qa, Cry8Ra, Cry8Sa, Cry8Ta, Cry9Aa, Cry9Ba, Cry9Bb, Cry9Ca, Cry9 Da, Cry9Db, Cry9Dc, Cry9Ea, Cry9Eb, Cry9Ec, Cry9Ed, Cry9Ee, Cry9Fa, Cry9Ga, Cry10Aa, Cry11Aa, Cry11Ba, Cry11Bb, Cry12Aa, Cry13Aa, Cry14Aa, Cry14Ab, Cry15Aa, Cry16Aa, Cry17Aa, Cry18Aa, Cry18Ba, Cry18Ca, Cry19Aa, Cry19Ba, Cry19Ca, Cry20Aa, Cry20Ba, Cry21Aa, Cry21Ba, Cry21Ca, Cry21 Da, Cry21Ea, Cry21Fa, Cry21Ga, Cry21Ha, Cry22Aa, Cry22Ab, Cry22Ba, Cry22Bb, Cry23Aa, Cry24Aa, Cry24Ba, Cry24Ca, Cry25Aa, Cry26Aa, Cry27Aa, Cry28Aa, Cry29Aa, Cry29Ba, Cry30Aa, Cry30Ba, Cry30Ca, Cry30 Da, Cry30Db, Cry30Ea, Cry30Fa, Cry30Ga, Cry31Aa, Cry31Ab, Cry31Ac, Cry31Ad, Cry32Aa, Cry32Ab, Cry32Ba, Cry32Ca, Cry32Cb, Cry32 Da, Cry32Ea, Cry32Eb, Cry32Fa, Cry32Ga, Cry32Ha, Cry32Hb, Cry32Ia, Cry32Ja, Cry32Ka, Cry32La, Cry32Ma, Cry32 Mb, Cry32Na, Cry32Oa, Cry32 Pa, Cry32Qa, Cry32Ra, Cry32Sa, Cry32Ta, Cry32Ua, Cry33Aa, Cry34Aa, Cry34Ab, Cry34Ac, Cry34Ba, Cry35Aa, Cry35Ab, Cry35Ac, Cry35Ba, Cry36Aa, Cry37Aa, Cry38Aa, Cry39Aa, Cry40Aa, Cry40Ba, Cry40Ca, Cry40 Da, Cry41Aa, Cry41Ab, Cry41Ba, Cry42Aa, Cry43Aa, Cry43Ba, Cry43Ca, Cry43Cb, Cry43Cc, Cry44Aa, Cry45Aa, Cry46Aa Cry46Ab, Cry47Aa, Cry48Aa, Cry48Ab, Cry49Aa, Cry49Ab, Cry50Aa, Cry50Ba, Cry51Aa, Cry52Aa, Cry52Ba, Cry53Aa, Cry53Ab, Cry54Aa, Cry54Ab, Cry54Ba, Cry55Aa, Cry56Aa, Cry57Aa, Cry57Ab, Cry58Aa, Cry59Aa, Cry59Ba, Cry60Aa, Cry60Ba, Cry61Aa, Cry62Aa, Cry63Aa, Cry64Aa, Cry65Aa, Cry66Aa, Cry67Aa, Cry68Aa, Cry69Aa, Cry69Ab, Cry70Aa, Cry70Ba, Cry70Bb, Cry71Aa, Cry72Aa, Cry73Aa, or any combination of the foregoing. A list of such Cry proteins and related information can be found, e.g., at the Bacterial Pesticidal Protein Resource Center (BPPRC) (Crickmore, N., Berry, C., Panneerselvam, S., Mishra, R., Connor, T. R. and Bonning, B. C. (2020). Bacterial Pesticidal Protein Resource Center, bpprc.org). Sequences are available through BPPRC and through the NCBI database (ncbi.nlm.nih.gov).

Non-limiting examples of members of the Vip3 class and their respective GenBank accession numbers, U.S. patent or patent publication number are Vip3Aa1 (AAC37036), Vip3Aa2 (AAC37037), Vip3Aa3 (see U.S. Pat. No. 6,137,033), Vip3Aa4 (AAR81079), Vip3Aa5 (AAR81080), Vip3Aa6 (AAR81081), Vip3Aa7 (AAK95326), Vip3Aa8 (AAK97481), Vip3Aa9 (CAA76665), Vip3Aa10 (AAN60738), Vip3Aa11 (AAR36859), Vip3Aa12 (AAM22456), Vip3Aa13 (AAL69542), Vip3Aa14 (AAQ12340), Vip3Aa15 (AAP51131), Vip3Aa16 (AAW65132), Vip3Aa17 (see U.S. Pat. No. 6,603,063), Vip3Aa18 (AAX49395), Vip3Aa19 (DQ241674), Vip3Aa19 (DQ539887), Vip3Aa20 (DQ539888), Vip3Aa21 (ABD84410), Vip3Aa22 (AAY41427), Vip3Aa23 (AAY41428), Vip3Aa24 (BI 880913), Vip3Aa25 (EF608501), Vip3Aa26 (EU294496), Vip3Aa27 (EU332167), Vip3Aa28 (FJ494817), Vip3Aa29 (FJ626674), Vip3Aa30 (FJ626675), Vip3Aa31 (FJ626676), Vip3Aa32 (FJ626677), Vip3Aa33 (GU073128), Vip3Aa34 (GU073129), Vip3Aa35 (GU733921), Vip3Aa36 (GU951510), Vip3Aa37 (HM132041), Vip3Aa38 (HM117632), Vip3Aa39 (HM117631), Vip3Aa40 (HM132042), Vip3Aa41 (HM132043), Vip3Aa42 (HQ587048), Vip3Aa43 (HQ594534), Vip3Aa44 (HQ650163), Vip3Ab1 (AAR40284), Vip3Ab2 (AAY88247), Vip3Ac1 (see U.S. Patent Application Publication 20040128716), Vip3Ad1 (see U.S. Patent Application Publication 20040128716), Vip3Ad2 (CAI43276), Vip3Ae1 (CAI43277), Vip3Af1 (see U.S. Pat. No. 7,378,493), Vip3Af2 (ADN08753), Vip3Af3 (HM117634), Vip3Ag1 (ADN08758), Vip3Ag2 (FJ556803), Vip3Ag3 (HM117633), Vip3Ag4 (HQ414237), Vip3Ag5 (HQ542193), Vip3Ah1 (DQ832323), Vip3Ba1 (AAV70653), Vip3Ba2 (HM117635), Vip3Bb1 (see U.S. Pat. No. 7,378,493), Vip3Bb2 (AB030520), and Vip3Bb3 (ADI48120).

In some embodiments, the insecticidal polypeptide can be a protein other than B. thuringiensis protein. For example, the insecticidal polypeptide can be an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin. In other embodiments, the insecticidal polypeptide can be at least one insecticidal protein from an insecticidal toxin complex (Tc) from Photorhabdus, Xenorhabus, Serratia, or Yersinia. In some embodiments, the insecticidal polypeptide can be an ADP-ribosyltransferase from an insecticidal bacteria, such as Photorhabdus ssp. In some embodiments, the insecticidal polypeptide can be a VIP protein, such as VIP1 and/or VIP2 from B. cereus. In some embodiments, the insecticidal polypeptide can be a binary toxin from an insecticidal bacteria, such as ISP1A and ISP2A from B. laterosporous or BinA and BinB from L. sphaericus. In some embodiments, the insecticidal polypeptide can be an engineered version or can be a hybrid or chimera of any of the preceding insecticidal polypeptides.

Other non-limiting example insecticidal polypeptides include DIG-657 (see US Patent Publication 2015366211); PtIP-96 (see US Patent Publication 2017233440); PIP-72 (see US Paten Publication US2016366891); PIP-83 (see US Patent Publication 2016347799); PIP-50 (see US Patent Publication 2017166921); IPD73 (see US Patent Publication 2019119334); IPD090 (see US Patent Publication 2019136258); IPD80 (see US Patent Publication 2019256563); IPD078, IPD084, IPD086, IPD087, IPD089 (see US Patent Publication 2020055906); IPD093 (see International Application Publication WO2018111551), IPD059 (see International Application Publication WO2018232072); IPD113 (see International Application Publication WO2019178042); IPD121 (see International Application Publication WO2018208882); IPD110 (see International Application Publication WO2019178038); IPD103 (see International Application Publication WO2019125717); IPD092; IPD095; IPD097; IPD099; IPD100, IPD105; IPD106; IPD107; IPD111; IPD112 (see International Application Publication WO2020055885); IPD102 (see International Application Publication WO2020076958) Cry1B.868 and Cry1 Da_7 (see US Patent Publication 2020-032289); TIC107 (see U.S. Pat. No. 8,049,071); Cry72Ab and Cry1A.105 (see U.S. patent Ser. No. 10/584,391): Cry1F, Cry34Ab1, Cry35Ab1 (see U.S. Pat. No. 10,407,688); TIC6757, TIC7472, TIC7473, TIC6757 (see US Patent Publication 2017058294); TIC3668, TIC3669, TIC3670 TIC4076, TIC4078, TIC4260, TIC4346, TIC4826, TIC4861, TIC4862, TIC4863, TIC-3668 (see US Patent Publication 2016319302); TIC7040, TIC7042, TIC7381, TIC7382, TIC7383, TIC7386, TIC7388, TIC7389 (see US Patent Publication 2018291305); TIC7941 (see US Patent Publication 2020229445) TIC836, TIC860, TIC867, TIC868, TIC869, and TIC1100 (see International Application Publication WO2016061391), TIC2160 (see International Application Publication WO2016061392), ET66, TIC400, TIC800, TIC834, TIC1415, AXMI-001, AXMI-002, AXMI-030, AXMI-035, AND AXMI-045 (see US Patent Publication 20130117884), AXMI-52, AXMI-58, AXMI-88, AXMI-97, AXMI-102, AXMI-112, AXMI-117, AXMI-100 (see US Patent Publication 201-0310543), AXMI-115, AXMI-113, AXMI-005 (see US Patent Publication 20130104259), AXMI-134 (see US Patent Publication 20130167264), AXMI-150 (see US Patent Publication 20100160231), AXMI-184 (see US Patent Publication 20100004176), AXMI-196, AXMI-204, AXMI-207, AXMI-209 (see US Patent Publication 2011-0030096), AXMI-218, AXMI-220 (see US Patent Publication 20140245491), AXMI-221z, AXMI-222z, AXMI-223z, AXMI-224z, AXMI-225z (see US Patent Publication 20140196175), AXMI-238 (see US Patent Publication 20140033363), AXMI-270 (see US Patent Publication 20140223598), AXMI-345 (see US Patent Publication 20140373195), AXMI-335 (see International Application Publication WO2013134523), DIG-3 (see US Patent Publication 20130219570), DIG-5 (see US Patent Publication 20100317569), DIG-11 (see US Patent Publication 20100319093), AfIP-1A (see US Patent Publication 20140033361), AfIP-1B (see US Patent Publication 20140033361), PIP-1APIP-1B (see US Patent Publication 20140007292), PSEEN3174 (see US Patent Publication 20140007292), AECFG-592740 (see US Patent Publication 20140007292), Pput_1063 (see US Patent Publication 20140007292), DIG-657 (see International Application Publication WO2015195594), Pput_1064 (see US Patent Publication 20140007292), GS-135 (see US Patent Publication 20120233726), GS153 (see US Patent Publication 20120192310), GS154 (see US Patent Publication 20120192310), GS155 (see US Patent Publication 20120192310), DIG-911 and DIG-180 (see US Patent Publication No. 20150264940); and the like.

In some embodiments, the putative insecticidal polypeptide comprises at least one modification relative to the wild type protein (i.e., the protein from which it is derived). In some embodiments, the modification is a result of DNA shuffling, block swapping, site-directed mutagenesis, saturation mutagenesis, random mutagenesis, or a combination thereof (e.g., as described in Cong et al., ed. Akhurst et al., 2002, Proceedings of the 4th Pacific Rim Conferences on Biotechnology of Bacillus thuringiensis and its environmental impact: 118-123, U.S. Pat. No. 8,530,411, Hou et al., 2019, Toxins 11(3):162, and U.S. Patent Application No. 20210147492). In some embodiments, the modification is a deletion of one or more nucleotides relative to the wild type protein. In some embodiments, the modification is an insertion of one or more nucleotides relative to the wild type protein. In some embodiments, the modification is a truncation, i.e., a deletion of one or more nucleotides from the 5′ end and/or the 3′ end of the wild type protein. In some embodiments, the putative insecticidal polypeptide is derived from a Bt Cry protein, and the modification relative to the wild type protein results in resistance to serine protease digestion (i.e., inhibition of serine protease activity). In some embodiments, serine protease inhibition potentiates the insecticidal activity of Bt Cry proteins (see, e.g., Pardo-López, et al., 2009, Peptides 30(3):589-595).

In some embodiments of the recombinant nucleic acids provided herein, the nucleic acid sequence encoding a putative insecticidal polypeptide comprises a nucleic acid sequence having at least 60% identity (e.g., at least 65% identity, at least 70% identity, at least 75% identity, at least 80% identity, at least 85% identity, at least 90% identity, at least 95% identity, or at least 99% identity) to SEQ ID NO:2 or SEQ ID NO:4. In some embodiments, the nucleic acid sequence encodes a putatitve insecticidal polypeptide comprising an amino acid sequence having at least 60% identity to SEQ ID NO:1 or SEQ ID NO:3.

One family of Cry proteins is known as the 3-domain Cry family and comprises proteins with three domains in the active toxin protein. These domains, referred to as domain I, domain II, and domain III, are described in, e.g., Bravo et al., 2008, Toxicon 49(4):423-435 and Palma et al., 2014, Toxins 6(12):3296-3325. Such Cry proteins can be expressed as protoxins comprising additional domains IV-VII, which are generally not required for toxicity. In some embodiments, the putative insecticidal polypeptides provided herein comprise domains I, II, and III. In some embodiments, the putative insecticidal polypeptides comprise domains I, II, III, and at least one of domains IV, V, VI, and VII.

### C. Insecticidal Protein Receptors

In some embodiments, the putative insecticidal polypeptides of the present disclosure are able to interact with a cell surface protein of a pest cell (e.g., an insect cell). Pests that can be targeted by the putative insecticidal proteins can include any of those listed below. In some embodiments, the pest cell is an epithelial cell. In some embodiments, the pest cell is part of pest digestive system. The cell surface proteins of the present disclosure can include any known receptor or any potential receptor of an insecticidal protein. In some embodiments, the cell surface protein is a receptor for any of the putative insecticidal proteins listed above. In some embodiments, the cell surface protein is a known Bt Cry protein receptor. In some embodiments, the cell surface protein is an ATP-Binding Cassette (ABC) transporter protein, a cadherin protein, an aminopeptidase N protein, or an alkaline phosphatase protein. In some embodiments, the cell surface protein is a VIP receptor such as, for example, VPAC1 and/or VPAC2. In some embodiments, the cell surface protein is a Scavenger Receptor (e.g., SR-C) (see, e.g., Wang et al., 2019, Appl. Environ. Microbiol. 85(16):e00579-19).

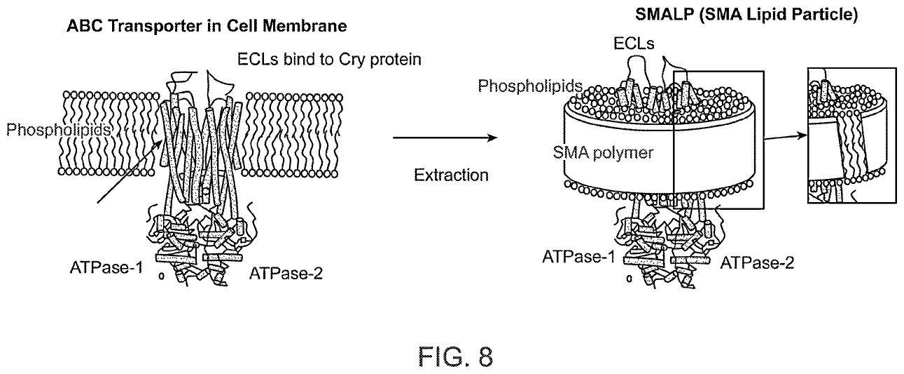

In some embodiments, the putative insecticidal polypeptides interact with an ABC transporter protein. Recently, ABC transporters have been identified as receptors of Bacillus thuringiensis (Bt) insecticidal proteins (Sato et al., 2019, Toxins 11:124 and Endo et al., 2018, J. Biol. Chem. 293:8569-8577). For example, a Bt insecticidal toxin called Cry1Fa (a Bt Cry insecticidal toxin) utilizes ABCC2 of Spodoptera frugiperda (Fall Armyworm) as its receptor to kill the insect. The Cry1Aa toxin binds to two of six extra cellular loops (ECLs) of Bm-ABCC2, ECL1 and ECL4, as described by Sato et al. (2019, Toxins 11:124). It is expected that Cry1Fa also binds to ECL1 and ECL4 of Sf-ABCC2 based on the structural similarity (FIG. 8). In some embodiments, as shown in FIG. 8, ABC transporter proteins have two membrane spanning alpha-helix bundles, where each bundle is made of 6 helices. In some embodiments, between the two bundles, there is a first ATPase (nucleotide binding) domain. In some embodiments, there is a second ATPase domain attached to the C′ terminal end of the last alpha-helix of the second alpha-helix bundle. In some embodiments, both ATPase domains are in the cytoplasm (i.e., on the intracellular side) (FIG. 8). In some embodiments, ABC transporter proteins without the second ATPase retain the same conformations of alpha-helix bundles and ECLs (i.e., relative to ABC transporter proteins with both ATPase domains). In some embodiments, the first ATPase domain (i.e., the ATPase domain in between the two alpha-helix bundles discussed above) is necessary to support the confirmation of ECL1 and ECL4. In some embodiments, the ABC transporter protein does not include the second ATPase domain.

In some embodiments, the ABC transporter can include a protein from any of the ABC transporter subfamilies, including ABCA, ABCB, ABCC, ABCD, ABCE, ABCF, ABCG, and ABCH. The ABC transporter protein can be from any of the pests listed below. In some embodiments, the ABC transporter protein is PxABCC2, Bm-ABCC2, Sf-ABCC2, Sf-ABCC3, Dv-ABCB1, Bm-ABCB1, Sf-ABCB1, Bm-ABCA2, or Tc-ABCC4. In some embodiments, the ABC transporter protein is PxABCC2, Bm-ABCC2, Sf-ABCC2, or Sf-ABCC3. In some embodiments, the ABC transporter protein retains ABC transporter function. In some embodiments, the ABC transporter protein does not retain ABC transporter function (e.g., ABC transporters without the second ATPase domain, as described above). In some embodiments, the ABC transporter protein comprises an amino acid sequence having at least 60% identity (e.g., at least 65% identity, at least 70% identity, at least 75% identity, at least 80% identity, at least 85% identity, at least 90% identity, at least 95% identity, or at least 99% identity) to any of SEQ ID NOs: 5, 7, 9, 11, or 13. In some embodiments, the ABC transporter protein is encoded by a nucleic acid sequence having at least 60% identity to any of SEQ ID NOs: 6, 8, 10, 12, or 14.

In some embodiments, the cell surface protein can be from a pest known to be resistant to one or more insecticides. In some embodiments, the cell surface protein is a mutant cell surface protein known to have reduced interaction with one or more insecticidal proteins of interest. For example, Cry1Fa-resistant colonies of S. frugiperda were found to have mutations in its ABCC2 to which Cry1Fa failed to bind (Banerjee et al., 2017, Sci. Rep. 7:10877). These reports indicate that the insecticidal proteins can be engineered to make it bind to a new receptor or a mutant of an existing receptor to overcome the resistance. For example, a new engineered protein that binds to the mutant of ABCC2 of Cry1Fa-resistant S. frugiperda may be able to overcome the resistance. Similarly, an engineered protein, which binds to S. frugiperda ABCC3, may be active to the Cry1Fa-resistant S. frugiperda. In some embodiments, the cell surface protein comprises a mutant form of an insecticidal protein receptor. In some embodiments, the cell surface protein comprises a mutant of ABCC2 from Cry1Fa-resistant S. frugiperda. In some embodiments, the Sf ABCC2 mutant comprises an amino acid sequence having at least 60% identity to SEQ ID NO:11. In some embodiments, the Sf ABCC2 mutant is encoded by a nucleic acid sequence having at least 60% identity to SEQ ID NO:12.

In some embodiments, the cell surface protein comprises one or more modifications relative to the wild type protein from which it is derived. In some embodiments, the modification is a result of DNA shuffling, block swapping, site-directed mutagenesis, saturation mutagenesis, random mutagenesis, or a combination thereof (e.g., as described in e.g., as described in Cong et al., ed. Akhurst et al., 2002, Proceedings of the 4th Pacific Rim Conferences on Biotechnology of Bacillus thuringiensis and its environmental impact: 118-123, U.S. Pat. No. 8,530,411, Hou et al., 2019, Toxins 11:162, and U.S. Patent Application No. 20210147492). In some embodiments, the modification is a deletion of one or more nucleotides relative to the wild type protein. In some embodiments, the modification is an insertion of one or more nucleotides relative to the wild type protein. In some embodiments, the modification is a truncation, i.e., a deletion of one or more nucleotides from the 5′ end and/or the 3′ end of the wild type protein.

### D. Target Pests

The insecticidal proteins provided herein can have activity against various target pests, including Lepidopteran pests, Coleopteran pests, Hemipteran pests, Dipteran pests, Lygus spp. Pests, and nematode pests.

In some embodiments, the insecticidal proteins can have activity against one or more of the following non-limiting examples of a Lepidopteran pest: Spodoptera spp. such as S. frugiperda (fall armyworm), S. littoralis (Egyptian cotton leafworm), S. ornithogalli (yellowstriped armyworm), S. praefica (western yellowstriped armyworm), S. eridania (southern armyworm), S. litura (Common cutworm/Oriental leafworm) and/or S. exigua (beet armyworm); Ostrinia spp. such as O. nubilalis (European com borer) and/or O. furnacalis (Asian corn borer); Plutella spp. such as P. xylostella (diamondback moth); Agrotis spp. such as A. ipsilon (black cutworm), A. segetum (common cutworm), A. gladiaria (claybacked cutworm), and/or A. orthogonia (pale western cutworm); Striacosta spp. such as S. albicosta (western bean cutworm); Helicoverpa spp. such as H. zea (corn earworm), H. punctigera (native budworm), and/or H. armigera (cotton bollworm); Heliothis spp. such as H. virescens (tobacco budworm); Diatraea spp. such as D. grandiosella (southwestern corn borer) and/or D. saccharalis (sugarcane borer); Trichoplusia spp. such as T. ni (cabbage looper); Sesamia spp. such as S. nonagroides (Mediterranean corn borer), S. inferens (Pink stem borer) and/or S. calamistis (pink stem borer); Pectinophora spp. such as P. gossypiella (pink bollworm); Cochylis spp. such as C. hospes (banded sunflower moth); Manduca spp. such as M. sexta (tobacco hornworm) and/or M. quinquemaculata (tomato hornworm); Elasmopalpus spp. such as E. lignosellus (lesser cornstalk borer); Pseudoplusia spp. such as P. includens (soybean looper); Anticarsia spp. such as A. gemmatalis (velvetbean caterpillar); Plathypena spp. such as P. scabra (green cloverworm); Pieris spp. such as P. brassicae (cabbage butterfly), Papaipema spp. such as P. nebris (stalk borer); Pseudaletia spp. such as P. unipuncta (common armyworm); Peridroma spp. such as P. saucia (variegated cutworm); Keiferia spp. such as K. lycopersicella (tomato pinworm); Artogeia spp. such as A. rapae (imported cabbageworm); Phthorimaea spp. such as P. operculella (potato tuberworm); Chrysodeixis spp. such as C. includens (soybean looper); Feltia spp. such as F. ducens (dingy cutworm); Chilo spp. such as C. suppressalis (striped stem borer), Cnaphalocrocis spp. such as C. medinalis (rice leaffolder), Conogethes spp. such as C. punctiferalis (Yellow peach moth), Mythimna spp. such as M. separata (Oriental armyworm), Athetis spp. such as A. lepigone (Two-spotted armyworm), or any combination of the foregoing.

In some embodiments, the insecticidal proteins can have activity against a coleopteran pest. In embodiments, the disclosed proteins have activity against Diabrotica spp. Diabrotica is a genus of beetles of the order Coleoptera commonly referred to as “corn rootworms” or “cucumber beetles.” Exemplary Diabrotica species include without limitation Diabrotica barberi (northern corn rootworm), D. virgifera virgifera (western corn rootworm), D. undecimpunctata howardii (southern corn rootworm), D. balteata (banded cucumber beetle), D. undecimpunctata undecimpunctata (western spotted cucumber beetle), D. significata (3-spotted leaf beetle), D. speciosa (chrysanthemum beetle), D. virgifera zeae (Mexican corn rootworm), D. beniensis, D. cristata, D. curviplustalata, D. dissimilis, D. elegantula, D. emorsitans, D. graminea, D. hispanloe, D. lemniscata, D. linsleyi, D. milleri, D. nummularis, D. occlusal, D. porrecea, D. scutellata, D. tibialis, D. trifasciata and D. viridula; and any combination thereof. Other nonlimiting examples of Coleopteran insect pests according to the present invention include Leptinotarsa spp. such as L. decemlineata (Colorado potato beetle); Chrysomela spp. such as C. scripta (cottonwood leaf beetle); Hypothenemus spp. such as H. hampei (coffee berry borer); Sitophilus spp. such as S. zeamais (maize weevil); Epitrix spp. such as E. hirtzpennis (tobacco flea beetle) and E. cucumeris (potato flea beetle); Phyllotreta spp. such as P. cruciferae (crucifer flea beetle) and P. pusilla (western black flea beetle); Anthonomus spp. such as A. eugenii (pepper weevil); Hemicrepidus spp. such as H. memnonius (wireworms); Melanotus spp. such as M. communis (wireworm); Ceutorhychus spp. such as C. assimiis (cabbage seedpod weevil); Phyllotreta spp. such as P. cruciferae (crucifer flea beetle); Aeolus spp. such as A. mellillus (wireworm); Aeolus spp. such as A. mancus (wheat wireworm); Horistonotus spp. such as H. uhlerii (sand wireworm); Sphenophorus spp. such as S. maidis (maize billbug), S. zeae (timothy billbug), S. parvulus (bluegrass billbug), and S. callosus (southern corn billbug); Phyllophaga spp. (White grubs); Chaetocnema spp. such as C. pulicaria (coin flea beetle); Popillia spp. such as P. japonica (Japanese beetle); Epilachna spp. such as E. varivestis (Mexican bean beetle); Cerotoma spp. such as C. trifurcate (Bean leaf beetle); Epicauta spp. such as F. pestifera and F. lemniscata (Blister beetles); and any combination of the foregoing.

The disclosed insecticidal proteins can be active against Hemipteran, Dipteran, Lygus spp., and/or other piercing and sucking insects, for example of the order Orthoptera or Thysanoptera. Insects in the order Diptera include but are not limited to any dipteran insect now known or later identified including but not limited to Liriomyza spp. such as L. trifolii (leafminer) and L. sativae (vegetable leafminer); Scrobipalpula spp. such as S. absoluta (tomato leafminer); Delia spp. such as D. platura (seedcorn maggot), D. brassicae (cabbage maggot) and D. radicum (cabbage root fly); Psilia spp. such as P. rosae (carrot rust fly); Tetanops spp. such as T. myopaeformis (sugarbeet root maggot); and any combination of the foregoing. Insects in the order Orthoptera include but are not limited to any orthopteran insect now known or later identified including but not limited to Melanoplus spp. such as M. differentialis (Differential grasshopper), M. femurrubrum (Redlegged grasshopper), M. bivittatus (Twostriped grasshopper); and any combination thereof. Insects in the order Thysanoptera include but are not limited to any thysanopteran insect now known or later identified including but not limited to Frankliniella spp. such as F. occidentalis (western flower thrips) and F. fusca (tobacco thrips); and Thrips spp. such as T. tabaci (onion thrips), T. palmi (melon thrips); and any combination of the foregoing.

The disclosed insecticidal proteins can be active against nematodes. The term “nematode” as used herein encompasses any organism that is now known or later identified that is classified in the animal kingdom, phylum Nematoda, including without limitation nematodes within class Adenophorea (including for example, orders Enoplida, Isolaimida, Mononchida, Dorylaimida, Trichocephalida, Mermithida, Muspiceida, Araeolaimida, Chromadorida, Desmoscolecida, Desmodorida and Monhysterida) and/or class Secementea (including, for example, orders Rhabdita, Strongylida, Ascaridida, Spirurida, Camallanida, Diplogasterida, Tylenchida and Aphelenchida). Nematodes include but are not limited to parasitic nematodes such as root-knot nematodes, cyst nematodes and/or lesion nematodes. Exemplary genera of nematodes according to the present invention include but are not limited to, Meloidogyne (root-knot nematodes), Heterodera (cyst nematodes), Globodera (cyst nematodes), Radopholus (burrowing nematodes), Rotylenchulus (reniform nematodes), Pratylenchus (lesion nematodes), Aphelenchoides (foliar nematodes), Helicotylenchus (spiral nematodes), Hoplolaimus (lance nematodes), Paratrichodorus (stubby-root nematodes), Longidorus, Nacobbus (false root-knot nematodes), Subanguina, Belonlaimus (sting nematodes), Criconemella, Criconemoides (ring nematodes), Ditylenchus, Dolichodorus, Hemicriconemoides, Hemicycliophora, Hirschmaniella, Hypsoperine, Macroposthonia, Melinius, Punctodera, Quinisulcius, Scutellonema, Xiphinema (dagger nematodes), Tylenchorhynchus (stunt nematodes), Tylenchulus, Bursaphelenchus (round worms), and any combination thereof. Exemplary plant parasitic nematodes according to the present disclosure include, but are not limited to, Belonolaimus gracihs, Belonolaimus longicaudatus, Bursaphelenchus xylophilus (pine wood nematode), Criconemoides ornata, Ditylenchus destructor (potato rot nematode), Ditylenchus dipsaci (stem and bulb nematode), Globodera pallida (potato cyst nematode), Globodera rostochiensis (golden nematode), Heterodera glycines (soybean cyst nematode), Heterodera schachtii (sugar beet cyst nematode); Heterodera zeae (corn cyst nematode), Heterodera avenae (cereal cyst nematode), Heterodera carotae, Heterodera trifolii, Hoplolaimus columbus, Hoplolaimus galeatus, Hoplolaimus magnistylus, Longidorus breviannulatus, Meloidogyne arenaria, Meloidogyne chitwoodi, Meloidogyne hapla, Meloidogyne incognita, Meloidogyne javanica, Mesocriconema xenoplax, Nacobbus aberrans, Naccobus dorsalis, Paratrichodorus christiei, Paratrichodorus minor, Pratylenchus brachyurus, Pratylenchus crenatus, Pratylenchus hexincisus, Pratylenchus neglectus, Pratylenchus penetrans, Pratylenchus projectus, Pratylenchus scribneri, Pratylenchus tenuicaudatus, Pratylenchus thornei, Pratylenchus zeae, Punctodera chaccoensis, Quinisulcius acutus, Radopholus similis, Rotylenchulus reniformis, Tylenchorhynchus dubius, Tylenchulus semipenetrans (citrus nematode), Szphmema americanum, X. mediterraneum, and any combination of the foregoing.

### E. Linkers

In some embodiments of the recombinant nucleic acids provided herein, the nucleic acid sequence encodes a linker. In some embodiments, the linker joins the spore outer coat polypeptide to the putative insecticidal polypeptide. In some embodiments, the spore outer coat polypeptide and the putative insecticidal polypeptide are structurally isolated by the linker. Polypeptides that are “structurally isolated” in a fusion protein, as described herein, are able to fold into their proper forms and thus maintain functionality. For example, an insecticidal polypeptide that is structurally isolated from a spore outer coat polypeptide is able to fold into its proper shape and maintain 1) its ability to bind to a target protein (e.g., an insect cell surface protein receptor) and 2) its insecticidal activity. Similarly, a spore outer coat polypeptide that is structurally isolated from a putative insecticidal protein is able to fold into its proper shape and maintain its ability to position the fusion protein in the outer coat of the spore. An important characteristic of the linkers described herein is the ability to keep the putative insecticidal polypeptide attached to the spore in which it is expressed. The linkers of the present disclosure can be optimized to produce desired effects (e.g., structural isolation) in the fusion proteins encoded by the recombinant nucleic acids. Aspects of linker design and considerations are described, for example, in Klein et al., 2014, Protein Eng. Des. Sel. 27(10):325-330. In some embodiments, the linker is resistant to protease digestion. In some embodiments, protease digestion resistance of the linker keeps the putative insecticidal polypeptide linked to the spore outer coat polypeptide (i.e., keeps the putative insecticidal protein attached to the surface of a spore in which it is expressed). In some embodiments, the linker is resistant to protease digestion in a protease-rich environment (e.g., a sporulated and/or lysed Bacillus culture medium).

In some embodiments, the linkers described herein comprise at least one amino acid. In some embodiments, the linker is a polypeptide comprising at least two amino acids. A linker sequence can increase the range of orientations that may be adopted by the polypeptides of the fusion protein. The peptide linker can be, for example, 1 to 100 or more amino acids in length (e.g., 1 aa, 2 aa, 3 aa, 4 aa, 5 aa, 10 aa, 15 aa, 20 aa, 25 aa, 30 aa, 35 aa, 40 aa, 45 aa, 50 aa, 55 aa, 60 aa, 65 aa, 70 aa, 75 aa, 80 aa, 85 aa, 90 aa, 95 aa, 100 aa, or more). Depending on length, linker sequence may have various conformations in secondary structure, such as helical, D-strand, coil/bend, and turns. In some instances, a linker sequence can have an extended conformation and function as an independent domain that does not interact with the adjacent protein domains. Linker sequences can be structurally flexible or structurally rigid. Flexible linkers provide a certain degree of movement or interaction between the polypeptide domains and are generally rich in small or polar amino acids such as Gly and Ser. A rigid linker can be used to keep a fixed distance between the domains and to help maintain their independent functions.

In some embodiments, the recombinant nucleic acids described herein encode a linker having at least 60% identity (e.g., at least 65% identity, at least 70% identity, at least 75% identity, at least 80% identity, at least 85% identity, at least 90% identity, at least 95% identity, or at least 99% identity) to any of the linkers in Table 1 or any of the linkers described in Klein et al., 2014, Protein Eng. Des. Set. 27(10):325-330. In some embodiments, the linker is a polypeptide comprising an amino acid sequence having at least 60% identity (e.g., at least 65% identity, at least 70% identity, at least 75% identity, at least 80% identity, at least 85% identity, at least 90% identity, at least 95% identity, or at least 99% identity) to any of SEQ ID Nos: 20, 21, or 38-61. In some embodiments, the linker comprises a sequence from a hydrophilic protein (e.g., maltose binding protein). In some embodiments, the linker comprises one or more repeats of the amino acid sequence GGS (SEQ ID NO:38), followed by an alanine residue and a serine residue. The number of repeats of GGGGS can be 1, 2, 3, 4, 5, 6, 7, 8, 9, 10, 11, 12, or more. In some embodiments, the linker comprises a Bt Cry protein leader sequence (e.g., the amino acid sequence of SEQ ID NO:20 or SEQ ID NO:21).

TABLE 1
Exemplary linker sequences.

| SEQ ID | |
|---|---|
| NO. | Linker sequence |
| 62 | AGSGGSGGSGGSPVPSTPPTNSSSTPPTPS |
| | PSPVPSTPPTNSSSTPPTPSPSPVPSTPPT |
| | NSSSTPPTPSPSAS |
| 39 | AGSGGSGGSGGSPVPSTPPTPSPSTPPTPS |
| | PSPVPSTPPTNSSSTPPTPSPSPVPSTPPT |
| | PSPSTPPTPSPSAS |
| 40 | AGSGGSGGSGGSPVPSTPPTPSPSTPPTPS |
| | PSGGSGNSSGSGGSPVPSTPPTPSPSTPPT |
| | PSPSAS |
| 41 | AGSGGSGGSGGSPVPSTPPTPSPSTPPTPS |
| | PSPVPSTPPTPSPSTPPTPSPSPVPSTPPT |
| | PSPSTPPTPSPSAS |
| 42 | AGSGGSGGSGGSPVPSTPPTPSPSTPPTPS |
| | PSIQRTPKIQVYSRHPAENGKSNFLNCYVS |
| | GFHPSDIEVDLLKNGERIEKVEHSDLSFSK |
| | DWSFYLLYYTEFTPTEKDEYACRVNHVTLS |
| | QPKIVKWDRDPVPSTPPTPSPSTPPTPSPS |
| | AS |
| 43 | AGSGGSGGSGGSPVPSTPPTPSPSTPPTPS |
| | PSIQRTPKIQVYSRHPAENGKSNFLNCYVS |
| | GFHPSDIEVDLLKNGERIEKVEHSDLSFSK |
| | DWSFYLLYYTEFTPTEKDEYACRVNHVTLS |
| | QPKIVKWDRDGGSGGSGGSGGSAS |
| 44 | AGPVPSTPPTPSPSTPPTPSPSIQRTPKIQ |
| | VYSRHPAENGKSNFLNCYVSGFHPSDIEVD |
| | LLKNGERIEKVEHSDLSFSKDWSFYLLYYT |
| | EFTPTEKDEYACRVNHVTLSQPKIVKWDRD |
| | GGSGGSGGSGGSIQRTPKIQVYSRHPAENG |
| | KSNFLNCYVSGFHPSDIEVDLLKNGERIEK |
| | VEHSDLSFSKDWSFYLLYYTEFTPTEKDEY |
| | ACRVNHVTLSQPKIVKWDRDGGSGGSGGSG |
| | AS |
| 45 | AGSGGSGGSGGSPVPSTPPTPSPSTPPTPS |
| | PSIQRTPKIQVYSRHPAENGKSNFLNCYVS |
| | GFHPSDIEVDLLKNGERIEKVEHSDLSFSK |
| | DWSFYLLYYTEFTPTEKDEYACRVNHVTLS |
| | QPKIVKWDRDGGSGGSGGSGGSIQRTPKIQ |
| | VYSRHPAENGKSNFLNCYVSGFHPSDIEVD |
| | LLKNGERIEKVEHSDLSFSKDWSFYLLYYT |
| | EFTPTEKDEYACRVNHVTLSQPKIVKWDRD |
| | PVPSTPPTPSPSTPPTPSPSAS |
| 46 | AGSGGSGGSGGSPVPSTPPTPSPSTPPTPS |
| | PSQIFVKTLTGKTITLEVEPSDTIENVKAK |
| | IQDKEGIPPDQQRLIFAGKQLEDGRTLSDY |
| | NIQKESTLHLVLRLRGGGGSGGSGGSGGSA |
| | S |
| 47 | AGSGGSGGSGGSPVPSTPPTPSPSTPPTPS |
| | PSDGRYSLTYIYTGLSKHVEDVPAFQALGS |
| | LNDLQFFRYNSKDRKSQPMGLWRQVEGMED |
| | WKQDSQLQKAREDIFMETLKDIVEYYNDSN |
| | GSHVLQGRFGCEIENNRSSGAFWKYYYDGK |
| | DYIEFNKEIPAWVPFDPAAQITKQKWEAEP |
| | VYVQRAKAYLEEECPATLRKYLKYSKNILD |
| | RQDPPSVVVTSHQAPGEKKKLKCLAYDFYP |
| | GKIDVHWTRAGEVQEPELRGDVLHNGNGTY |
| | QSWVVVAVPPQDTAPYSCHVQHSSLAQPLV |
| | VPWEASPVPSTPPTPSPSTPPTPSAS |
| 48 | AGSGGSGGSGGSGGSGGSGGSGGSDGRYSL |
| | TYIYTGLSKHVEDVPAFQALGSLNDLQFFR |
| | YNSKDRKSQPMGLWRQVEGMEDWKQDSQLQ |
| | KAREDIFMETLKDIVEYYNDSNGSHVLQGR |
| | FGCEIENNRSSGAFWKYYYDGKDYIEFNKE |
| | IPAWVPFDPAAQITKQKWEAEPVYVQRAKA |
| | YLEEECPATLRKYLKYSKNILDRQDPPSVV |
| | VTSHQAPGEKKKLKCLAYDFYPGKIDVHWT |
| | RAGEVQEPELRGDVLHNGNGTYQSWVVVAV |
| | PPQDTAPYSCHVQHSSLAQPLVVPWEASGG |
| | SGGSGGSGGSDGRYSLTYIYTGLSKHVEDV |
| | PAFQALGSLNDLQFFRYNSKDRKSQPMGLW |
| | RQVEGMEDWKQDSQLQKAREDIFMETLKDI |
| | VEYYNDSNGSHVLQGRFGCEIENNRSSGAF |
| | WKYYYDGKDYIEFNKEIPAWVPFDPAAQIT |
| | KQKWEAEPVYVQRAKAYLEEECPATLRKYL |
| | KYSKNILDRQDPPSVVVTSHQAPGEKKKLK |
| | CLAYDFYPGKIDVHWTRAGEVQEPELRGDV |
| | LHNGNGTYQSWVVVAVPPQDTAPYSCHVQH |
| | SSLAQPLVVPWEASPVPSTPPTPSPSTPPT |
| | PSPSAS |
| 49 | AGSGNSSGSGGSGGSGNSSGSGGSPVPSTP |
| | PTPSPSTPPTPSPSAS50KLSGGGGSGGGG |
| | SGGGGSAEAWYNLGNAYYKQGDYQKAIEYY |
| | QKALELDPNNAEAWYNLGNAYYKQGDYQKA |
| | IEYYQKALELDPNNAEAWYNLGNAYYKQGD |
| | YQKAIEDYQKALELDPNNLQRSAGGGGSGG |
| | GGSGGGGAS |
| 51 | KLSGGGGGGGGSGGGGSAEAWYNLGNAYYK |
| | QGDYQKAIEYYQKALELDPNNAEAWYNLGN |
| | AYYKQGDYQKAIEYYQKALELDPNNAEAWY |
| | NLGNAYYKQGDYQKAIEDYQKALELDPNNL |
| | QAEAWKNLGNAYYKQGDYQKAIEYYQKALE |
| | LDPNNASAWYNLGNAYYKQGDYQKAIEYYQ |
| | KALELDPNNAKAWYRRGNAYYKQGDYQKAI |
| | EDYQKALELDPNNRSRSAGGGGSGGGGSGG |
| | GGAS |
| 52 | KLSGGGGSGGGGSGGGGSAEAWYNLGNAYY |
| | KQGDYQKAIEYYQKALELDPNNAEAWYNLG |
| | NAYYKQGDYQKAIEYYQKALELDPNNAEAW |
| | YNLGNAYYKQGDYQKAIEDYQKALELDPNN |
| | LQAEAWKNLGNAYYKQGDYQKAIEYYQKAL |
| | ELDPNNASAWYNLGNAYYKQGDYQKAIEYY |
| | QKALELDPNNAKAWYRRGNAYYKQGDYQKA |
| | IEDYQKALELDPNNRSAEAWYNLGNAYYKQ |
| | GDYQKAIEYYQKALELDPNNAEAWYNLGNA |
| | YYKQGDYQKAIEYYQKALELDPNNAEAWYN |
| | LGNAYYKQGDYQKAIEDYQKALELDPNNLQ |
| | RSAGGGGSGGGGSGGGGAS |
| 53 | KLSGGGGSGGGGSGGGGSAEAWYNLGNAYY |
| | KQGDYQKAIEYYQKALELDPNNAEAWYNLG |
| | NAYYKQGDYQKAIEYYQKALELDPNNAEAW |
| | YNLGNAYYKQGDYQKAIEDYQKALELDPNN |
| | LQAEAWKNLGNAYYKQGDYQKAIEYYQKAL |
| | ELDPNNASAWYNLGNAYYKQGDYQKAIEYY |
| | QKALELDPNNAKAWYRRGNAYYKQGDYQKA |
| | IEDYQKALELDPNNRSAEAWYNLGNAYYKQ |
| | GDYQKAIEYYQKALELDPNNAEAWYNLGNA |
| | YYKQGDYQKAIEYYQKALELDPNNAEAWYN |
| | LGNAYYKQGDYQKAIEDYQKALELDPNNLQ |
| | AEAWKNLGNAYYKQGDYQKAIEYYQKALEL |
| | DPNNASAWYNLGNAYYKQGDYQKAIEYYQK |
| | ALELDPNNAKAWYRRGNAYYKQGDYQKAIE |
| | DYQKALELDPNNRSAGGGGSGGGGSGGGGA |
| | S |
| 54 | GGGGSAS |
| 55 | GGGGSGGGGSAS |
| 56 | GGGGSGGGGSGGGGSAS |
| 57 | GGGGSGGGGSGGGGSGGGGSGGGGSAS |
| 58 | GGGGSGGGGSGGGGSGGGGSGGGGSGGGGS |
| | AS |
| 59 | AGGGSGGGGSGGGGSGGGGSGGGGSGGGGS |
| | GGGGSAS |
| 60 | AGGGSGGGGSGGGGSGGGGSGGGGSGGGGS |
| | GGGGSGGGGSAS |
| 61 | AGGGSGGGGSGGGGSGGGGSGGGGSGGGGS |
| | GGGGSGGGGSGGGGSAS |

### F. DNA Constructs and Vectors

Also provided herein are DNA constructs comprising a promoter operably linked to any of the recombinant nucleic acids described herein. A nucleic acid is “operably linked” when it is placed into a functional relationship with another nucleic acid sequence. Numerous promoters can be used in the constructs described herein. A promoter is a region or a sequence located upstream and/or downstream from the start of transcription that is involved in recognition and binding of RNA polymerase and other proteins to initiate transcription. The promoter can be a eukaryotic or a prokaryotic promoter. In some embodiments the promoter is an inducible promoter. In some embodiments, the promoter is a constitutive promoter. In some embodiments, the promoter is an endogenous promoter for a spore outer coat polypeptide gene (i.e., the promoter driving expression of the gene in its natural genomic context). In some embodiments, the promoter is the endogenous promoter for the particular spore outer coat polypeptide gene encoded by the recombinant nucleic acid. In some embodiments, the promoter is an endogenous CotC or CotG promoter. In some embodiments, the promoter is a CotA, CotB, CotD, CotE, CotX, CotY, or CotZ promoter. In some embodiments, the promoter is a Bacillus species promoter. In some embodiments, the promoter is under sigma K factor control in Bacillus species.

In some embodiments, the promoter is a sporulation-specific promoter. Genes involved in spore synthesis and structure have been identified and cloned, and promoter sequences from such genes have been isolated and characterized. One of skill in the art will appreciate that by selecting among these promoters and regulatory sequences, it is possible to govern the physical location of expression of the polypeptide of interest in the spore or vegetative cell as well as the timing of expression in the life cycle of the spore and/or vegetative cell. See, for example, Hill et al., Soc. Appl. Bacteriol. Symp. Ser. 23: 129S-134S (1998), demonstrating that model proteins such as luciferase and beta-galactosidase can be directed to the endospore during the sporulation process by operably linking the nucleic acid sequences encoding these proteins to sporulation-specific promoters. Hill et al. further demonstrates that these model enzymes are stored effectively and retain their activities. See also U.S. Patent Application Publication 20030165538.

The recombinant nucleic acids provided herein can be included in expression cassettes for expression in a host cell or an organism of interest. The cassette can include 5′ and 3′ regulatory sequences operably linked to a recombinant nucleic acid provided herein that allows for expression of the fusion protein. The cassette can additionally contain at least one additional gene or genetic element to be cotransformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory regions. The expression cassette can include in the 5′ to 3′ direction of transcription: a transcriptional and translational initiation region (i.e., a promoter), a recombinant nucleic acid disclosed herein, and a transcriptional and translational termination region (i.e., termination region) functional in the cell or organism of interest. The promoters described herein are capable of directing or driving expression of a coding sequence in a host cell. The regulatory regions (i.e., promoters, transcriptional regulatory regions, and translational termination regions) can be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention.

Additional regulatory signals include, but are not limited to, transcriptional initiation start sites, operators, activators, enhancers, other regulatory elements, ribosomal binding sites, an initiation codon, termination signals, and the like. See Sambrook et al. (1992) Molecular Cloning: A Laboratory Manual, ed. Maniatis et al. (Cold Spring Harbor Laboratory Press, Cold Spring Harbor, N.Y.), Davis et al., eds. (1980) Advanced Bacterial Genetics (Cold Spring Harbor Laboratory Press), Cold Spring Harbor, N.Y., and the references cited therein.

The expression cassette can also comprise a selectable marker gene for the selection of transformed cells. Marker genes include genes conferring antibiotic resistance, such as those conferring hygromycin resistance, ampicillin resistance, gentamicin resistance, neomycin resistance, to name a few. Additional selectable markers are known and any can be used.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Further provided herein are vectors comprising a recombinant nucleic acid or expression cassette set forth herein. The vector is contemplated to have the necessary functional elements that direct and regulate transcription of the inserted nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers that can serve to select for cells containing the vector or the vector containing the insert, RNA splice junctions, a transcription termination region, or any other region that may serve to facilitate the expression of the inserted gene or hybrid gene (See generally, Sambrook et al. Molecular Cloning: A Laboratory Manual, 4^{th} ed., Cold Spring Harbor Laboratory Press, Cold Spring Harbor, 2012). The vector, for example, can be a plasmid.

A host cell comprising a recombinant nucleic acid, a DNA construct, or a vector described herein is also provided. In some embodiments, the host cell is a bacterial cell or a fungal cell. The host cell can be any spore-producing microbial cell, including, for example, a cell from any of the bacterial or fungal species listed above. The host cell can be an in vitro, ex vivo, or in vivo host cell. Populations of any of the host cells described herein are also provided. A cell culture comprising one or more host cells described herein is also provided. Methods for the culture and production of many cells, including cells of bacterial (for example E. coli and other bacterial strains) origin are available in the art. See e.g., Sambrook (supra), Ausubel et al., (2010) Current protocols in molecular biology, John Wiley and Sons, New York, and Berger and Kimmel (1987) Guide to Molecular Cloning Techniques, Methods in Enzymology 152:3-812, as well as Freshney (1994) Culture of Animal Cells, a Manual of Basic Technique, 3^{rd} Ed., Wiley-Liss, NY and the references cited therein; Doyle and Griffiths (1997) Mammalian Cell Culture: Essential Techniques John Wiley and Sons, NY; Humason (1979) Animal Tissue Techniques, 4^{th} Ed. W.H. Freeman and Company; and Ricciardelli, et al., (1989) In vitro Cell Dev. Biol. 25:1016-1024.

As used herein, the phrase “introducing” in the context of introducing a nucleic acid into a cell refers to the translocation of the nucleic acid sequence from outside a cell to inside the cell. Various methods of such translocation are contemplated, including but not limited to, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (CRISPR-Cas9), a transcription activator-like effector nuclease (TALEN), a zinc finger nuclease (ZFN), or a megaTAL (MT) (Li et al. Signal Transduction and Targeted Therapy 5, Article No. 1 (2020)) can also be used to introduce a nucleic acid, for example, a nucleic acid encoding a recombinant protein described herein, into a host cell.

### G. Fusion Proteins

Also provided herein are fusion proteins encoded by any of the recombinant nucleic acids, DNA constructs, or vectors described herein.

Any of the polypeptides or proteins described herein can further comprise a detectable moiety, for example, a fluorescent protein or fragment thereof. Examples of fluorescent proteins include, but are not limited to, yellow fluorescent protein (YFP, for example, Venus), green fluorescent protein (GFP), and red fluorescent protein (RFP) as well as derivatives, for example, mutant derivatives, of these proteins. See, for example, Chudakov et al. “Fluorescent Proteins and Their Applications in Imaging Living Cells and Tissues,” Physiological Reviews 90(3): 1103-1163 (2010); and Specht et al., “A Critical and Comparative Review of Fluorescent Tools for Live-Cell Imaging,” Annual Review of Physiology 79: 93-117 (2017).

Any of the polypeptides or proteins described herein can further comprise a domain or sequence useful for protein isolation. In some embodiments, the polypeptides comprise an affinity tag, for example a FLAG tag (SEQ ID NO:24), a Myc tag (EQKLISEEDL (SEQ ID NO:26)), a polyhistidine tag (e.g., 8×His tag (SEQ ID NO:27)), an albumin-binding protein, an alkaline phosphatase, an AU1 epitope, an AU5 epitope, a biotin-carboxy carrier protein (BCCP), to name a few. In some embodiments, the affinity tags are useful for protein isolation. See, Kimple et al. “Overview of Affinity Tags for Protein Purification,” Curr. Protoc. Protein Sci. 73: Unit-9.9 (2013). In some embodiments, the polypeptides or proteins described herein comprise a signal sequence useful for protein isolation. See, for example, Low et al. “Optimisation of signal peptide for recombinant protein secretion in bacterial hosts,” Applied Microbiology and Biotechnology 97:3811-3826 (2013). In some embodiments, the polypeptides or proteins described herein comprise a protease recognition site. Such protease recognition sites may be useful for, among other things, allowing removal of a signal peptide or affinity purification tag following protein isolation.

### H. Spores

Also provided herein are microbial spores comprising any of the recombinant nucleic acids, DNA constructs, vectors, or fusion proteins described herein. In some embodiments, the fusion protein is expressed as a component of the spore. In some embodiments, the fusion protein is displayed on the surface of the spore.

In some embodiments, the spore is a bacterial spore or a fungal spore. In some embodiments, the spore is from any of the spore-producing microbes listed in Section A of this disclosure. In some embodiments, the spore is from a Bacillus species that is not known to produce any insecticidal protein in its spores.

Also provided herein are compositions comprising a mixture of spores, wherein at least two of the spores in the mixture comprise different putative insecticidal polypeptides. In some embodiments, the composition comprises a plurality of the spores described herein, wherein the plurality comprises spores comprising recombinant nucleic acids encoding different putative insecticidal polypeptide. For example, the mixture or plurality of spores can comprise spores comprising a first recombinant nucleic acid encoding a first putative insecticidal polypeptide and spores comprising a second recombinant nucleic acid encoding second putative insecticidal polypeptide. The mixture or plurality of spores can comprise spores encoding a wide range of putative insecticidal polypeptides. In some embodiments, the mixture or plurality of spores comprises spores comprising up to 10×10\hat{ }8 different putative insecticidal polypeptides (e.g., up to 10×10\hat{ }7, up to 10×10\hat{ }6, or up to 10×10\hat{ }5). In some embodiments, the composition comprises a plurality of the spores described herein, wherein each spore in the plurality comprises a recombinant nucleic acid encoding a different putative insecticidal polypeptide.

### IV. Methods of Identifying Insecticidal Proteins and Insecticidal Protein Receptors

In another aspect, provided herein are methods of screening for and identifying insecticidal proteins. In some embodiments, the methods comprise display of an insecticidal protein library on bacterial or fungal spores (e.g., spores of Bacillus species) and screening against one or more receptor proteins. In some embodiments, the methods comprise contacting a fusion protein or a spore comprising a fusion protein as described above with an insecticidal protein receptor as described above. In some embodiments, the insecticidal protein receptor protein is an ABC transporter protein. In some embodiments, the methods comprise detecting an interaction between the fusion protein or spore and the insecticidal protein receptor. In some embodiments, such an interaction indicates that the putative insecticidal protein of the fusion protein or spore is an insecticidal protein.

In some embodiments, the fusion protein or spore is contacted with a purified insecticidal protein receptor protein. In some embodiments, the fusion protein or spore is contacted with a crude extract comprising an insecticidal protein receptor protein. In some embodiments, the fusion protein or spore is contacted with the insecticidal protein receptor in an environment simulating a relevant biological environment. For example, the environment simulated can be an insect midgut environment. The environment can be a neutral or alkaline pH (i.e., a pH level of 7.0 or higher) environment, such as a buffered solution. In some embodiments, the pH is maintained at 7.5 pH using Tris buffered saline (TBS). In some embodiments, the pH is maintained at 8.0 pH to 11.0 pH. For example, the pH can be maintained at up to 11.0 pH (e.g., at 11 pH, at 10.5 pH, at 10 pH, at 9.5 pH, at 9 pH, at 8.5 pH, or at 8 pH). Suitable alkaline buffers (e.g., a buffer with a pKa value at or above 8.2) are known in the art, and include but are not limited to CAPS, CABS, cholamine chloride, MOPS, BES, TES, HEPES, DIPSO, MOBS, acetamidoglycine, TAPSO, TEA, POPSO, HEPPSO, EPS, HEPPS, tricine, tris, glycinamide, glycylglycine, HEPBS, bicine, TAPS, AMPB, CHES, CAPSO, and AMP). In some embodiments, the pH is maintained at around 10 pH using CAPS or CABS buffers.

In some embodiments, the insecticidal protein receptor is a membrane protein. Specialized production and/or isolation methods may be used to purify membrane proteins (i.e., to maintain the structure and/or function of the membrane protein). In some embodiments, the protein is purified as part of a copolymer. For example, in some embodiments, the insecticidal protein receptor is purified as part of a styrene maleic acid (SMA) lipid particle (SMALP) (e.g., according to the methods described in Example 7 herein). In some embodiments, the receptor protein is purified as part of an SMA copolymer with a particular S:M ratio (i.e., the ratio of styrene to maleic-anhydride in the copolymer). This ratio may be determined via experimentation by one of skill in the art. In some embodiments, the S:M ratio for purification of an ABC transporter protein is between 2:1 and 3:1, inclusive (e.g., 2:1, 2.1:1, 2.2:1, 2.3:1, 2.4:1, 2.5:1, 2.6:1, 2.7:1, 2.8:1, 2.9:1, or 3:1). Preparing insecticidal protein receptor proteins (e.g., an ABC transporter protein) in a SMALP can preserve the structure and function of the receptor protein. Use of SMALP-ABC transporter proteins in Cry binding assays has not been previously described. Previously, detergents such as DDM have been used to purify ABC transporters for Cry binding assays. Without being bound to any particular theory, it is likely the structure and function of ABC transporter is affected negatively by detergents, which replace or strip insect phospholipids that support the structure of ABC transporter.

The insecticidal protein receptor proteins of the present disclosure can be expressed in and/or purified from any suitable cell (e.g., insect cells, prokaryotic cells, eukaryotic cells, etc.). In some embodiments, the receptor proteins are expressed in insect cells. In some embodiments, the receptor proteins are expressed from baculovirus expression vectors. Recombinant proteins produced in insect cells with baculovirus vectors undergo post-translational modifications. Suitable vectors are known to one of ordinary skill in the art. Insect cells useful for expression of the receptor proteins herein include Sf9 cells, Sf21 cells, silkworm pupae cells, or other insect cell lines known to one of ordinary skill in the art. Other cell that may be useful for expression of the receptor proteins herein include Expi293 cells and HEK293 cells. See Willcoxon et al., 2016, J. Biotech 217:72-81 and Niu et al., 2020, Sci. Reports 2020(10):15830.

In some embodiments, the receptor proteins are expressed in and purified from prokaryotic cells. There are numerous E. coli expression vectors known to one of ordinary skill in the art, which are useful for the expression and purification of a receptor protein. Other microbial hosts suitable for use include bacilli, such as Bacillus subtilis, and other enterobacteriaceae, such as Salmonella, Senatia, and various Pseudomonas species. In these prokaryotic hosts, one can also make expression vectors, which typically contain expression control sequences compatible with the host cell (e.g., an origin of replication). In addition, a promoter is operably linked to the sequence encording the receptor protein. Exemplary well-known promoters include the lactose promoter system, a tryptophan (Trp) promoter system, a beta-lactamase promoter system, and a promoter system from phage lambda. Additionally, yeast expression can be used, e.g., the receptor protein can be expressed by Pichia pastoris or S. cerevisiae.

Mammalian cells also permit the expression and purification of proteins. Vectors useful for the expression of active proteins in mammalian cells are known in the art and can contain genes conferring hygromycin resistance, geneticin or G418 resistance, or other genes or phenotypes suitable for use as selectable markers, or methotrexate resistance for gene amplification. A number of suitable host cell lines capable of secreting intact proteins have been developed in the art, and include CHO cells, HeLa cells, HEK-293 cells, HEK-293T cells, U2OS cells, or any other primary or transformed cell line. Other suitable host cell lines include COS-7 cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences.

Expression vectors for expression of the proteins described herein can also include nucleic acids encoding the proteins as described herein under the control of an inducible promoter such as the tetracycline inducible promoter or a glucocorticoid inducible promoter. The nucleic acids can also be under the control of a tissue-specific promoter to promote expression of the nucleic acid in specific cells, tissues or organs. Any regulatable promoter, such as a metallothionein promoter, a heat-shock promoter, and other regulatable promoters, of which many examples are well known in the art are also contemplated. Furthermore, a Cre-loxP inducible system can also be used, as well as a Flp recombinase inducible promoter system, both of which are known in the art.

In some embodiments of the methods provided herein, the fusion protein or spore is contacted with an insecticidal protein receptor on the surface of a cell (i.e., rather than a purified protein or a protein present in a cell lysate). In some embodiments, the cell is an insect cell (e.g., from any of the insects listed in Section D: Target pests above). In some embodiments, the cell is part of a cell culture. In some embodiments, the cell is present in a live insect.

In some embodiments, detecting the interaction between the fusion protein or spore and the insecticidal protein receptor comprises performing immuno-magnetic separation, flow cytometry, cytotoxicity assays, sequencing, or a combination thereof. In some embodiments, an epitope on the insecticidal protein receptor protein (e.g., a FLAG epitope) is used with an antibody against the epitope to purify insecticidal protein receptors (e.g., via immuno-magnetic separation). When a fusion protein or spore has interacted with a receptor, it will be purified along with the receptor protein. In some embodiments, spores that interact with a receptor protein can be identified via flow cytometry. In some embodiments, spores that interact with a receptor protein can be identified via a cytotoxicity assay (e.g., as described in Example 5 herein).

In some embodiments of the methods provided herein, because the putative insecticidal polypeptide is tethered to a spore (e.g., via fusion to the spore outer coat polypeptide), the putative insecticidal polypeptide can be identified after the bound spores (i.e., spores which have interacted with a receptor protein) have been sorted. This aspect enables high-throughput screening and differentiates the methods herein from previous insect-active toxin screening methods, in which putative insecticidal proteins had to be produced individually in host microbes (e.g., in 96-well plates where each well has host cells expressing a different putative insecticidal protein) and tested separately against receptor proteins.

In some embodiments, detecting the interaction between the fusion protein or spore and the insecticidal protein receptor further comprises isolating the recombinant nucleic acid encoding the fusion protein that interacts with the insecticidal protein receptor protein and performing sequencing to determine the identity of the insecticidal protein.

In some embodiments, the methods provided herein comprise the following steps (e.g., as demonstrated in the Examples herein), though while numbered, certain steps can be performed in a different order than listed and/or concurrently:
1. A library of diversified insecticidal proteins is made by employing various protein engineering technologies and isolating naturally occurring insecticidal proteins;
2. The library of Step 1 is cloned in a spore display vector to display the proteins on the surface of spores;
3. A protein sample of the desired receptor (e.g., ABCC2-mutant from Cry1Fa-resistant S. frugiperda colonies to overcome the resistance) is produced in a receptor expression system (e.g., insect cell line such as Sf9 or live insects such as silkworm using a baculovirus expression system);
4. The receptor protein is extracted from the cell membrane such that its native protein structure is preserved and purified for the next step;
5. Spores produced from Step 2 will be screened for the capability of binding to the receptor produced in Step 4 using a cell sorting technology such as immuno-magnetic separation and flow cytometry;
6. Steps 1-5 above are repeated until one or more insecticidal protein(s) are obtained.

In some embodiments, provided herein is a method of identifying an insecticidal protein capable of binding to a specific insecticidal protein receptor, wherein the receptor is expressed on the surface of insect cells, the method comprising: (a) displaying a library of putative insecticidal proteins on the surface of microbial endospores from a spore display vector cloned in the host microbe in an orientation such that the receptor binding moiety of the putative insecticidal protein is exposed to the solvent and capable of binding to insect cell receptor protein; (b) isolating and preparing an insecticidal protein receptor protein (i.e., a toxin receptor) in a form that retains the native receptor protein structure and is capable of binding to the putative insecticidal protein; (c) sorting the putative insecticidal-carrying spores using a cell (particle) sorting technique (e.g., flow cytometry or magnetic sorting), based on the binding capability of the putative insecticidal protein to the insect cell membrane protein prepared in (b); (d) optionally determining the gene sequence of the putative insecticidal proteins tethered on spores that bind to the receptor protein; and, optionally, (e) determining the cytotoxicity of toxin proteins on the spores sorted in (c) using cultured cells such as Sf9 and HEK293 in which the toxin receptor is cloned and expressed.

Also contemplated herein are methods of identifying insecticidal protein receptors for a particular insecticidal protein. In some embodiments, a fusion protein or spore (e.g., as described herein) comprising a known or putative insecticidal polypeptide can be contacted with a library of receptor proteins. In some embodiments, the library includes known receptors or engineered synthetic receptors (e.g., produced via DNA shuffling, block swapping, site-directed mutagenesis, saturation mutagenesis, random mutagenesis, or a combination thereof). In some embodiments, interaction between the fusion protein or spore is detected as described above (e.g., using an epitope on the fusion protein in immune-magnetic separation). In some embodiments, such an interaction indicates that the receptor protein is a receptor for the insecticidal polypeptide of the fusion protein or spore.

### V. Methods for Pest Control

Also contemplated herein are methods of using the fusion proteins or spores described herein as biological agents for pest control. As used herein, “pest control” refers to reduction of a negative impact of a pest on a plant. A pest or plant pest is a species, strain, or biotype of plant, animal, or pathogenic agent that directly or indirectly injures, causes damage to, or causes disease to plants or plant products. In the context of this disclosure, a “pest” is an insect pest. In some embodiments, the pest is any of the pests listed in Section III.D above. In some embodiments, pest control is achieved by biocidal activity (i.e., by killing the pest). With regards to insect pests, biocidal activity can be referred to as insecticidal activity. In some embodiments, pest control is achieved by reducing the impact of a pest on the plant.

In some embodiments, a composition comprising a fusion protein or spore described herein is applied to a plant, a plant seed, or a plant propagation material (e.g., a seed, a leaf, a stem, a root, or any other plant tissue). In some embodiments, the composition comprises bacteria that produce a spore described herein. In some embodiments, the composition includes other insecticidal agents known in the art. In some embodiments, the composition comprises additional auxiliary components conventionally used in the art, such as, for example, excipients, stabilizers, carriers, dispersants, fertilizers, nutrients, growth additives, stabilizers, “slow release” auxiliaries, colourants and, if appropriate, surface-active substances (surfactants). Suitable auxiliary components include all those substances which are conventionally used for crop protection products.

In some embodiments, the compositions comprise one or more excipients. The one or more excipients can be one or more stabilizers, one or more additives, one or more carriers, one or more dispersants, one or more fertilizers, or any combination thereof. In one example, one or more excipients comprise acetone.

In some embodiments, the compositions comprise one or more stabilizers and/or other additives. The stabilizers and/or additives can include, but are not limited to, penetration agents, adhesives, anticaking agents, dyes, dispersants, wetting agents, emulsifying agents, defoamers, antimicrobials, antifreeze, pigments, colorants, buffers, and carriers. The compositions may further comprise surfactants and/or adjuvants.

In some embodiments, the compositions disclosed herein may further comprise one or more carriers. Examples of carriers include, but are not limited to, solid carriers, sponges, textiles, and synthetic materials. The synthetic material may be a porous synthetic material. Additional carriers can include organic carriers, such as waxes, linolin, paraffin, dextrose granules, sucrose granules and maltose-dextrose granules. Alternatively, the carrier can be an anorganic carrier such as natural clays, kaolin, pyrophyllite, bentonite, alumina, montmorillonite, kieselguhr, chalk, diatomaceous earths, calcium phosphates, calcium and magnesium carbonates, sulphur, lime, flours or talc. The composition may be adsorbed into the carrier.

In some embodiments, the compositions comprise one or more dispersants. The dispersant may be a negatively charged anion dispersant. The dispersant may be a nonionic dispersant.

In some embodiments, the compositions comprise a fertilizer, nutrient, or other growth additive. The fertilizer may be a chemical fertilizer. The fertilizer may be an organic fertilizer. The fertilizer may be an inorganic fertilizer. The fertilizer may be a granulated or powdered fertilizer. The fertilizer may be a liquid fertilizer. The fertilizer may be a slow-release fertilizer.

In some embodiments, the pest control methods provided herein comprise treating a plant, a plant seed, or a plant propagation material with a pest control-effective amount of a fusion protein or spore described herein (or a composition comprising a fusion protein or spore described herein). A “pest control-effective amount,” as used herein, refers to an amount of a fusion protein, spore, or composition capable of killing, controlling, or infecting the target pest, retarding the growth or reproduction of the target pest, reducing the population of the target pest, and/or reducing damage to plants caused by the target pest. The pest control-effective amount of a given fusion protein, spore, or composition will vary, depending upon factors including, but not limited to, the plant species, the surface area of the plant, plant seed, or plant propagation material, the type of carrier, presence or absence of other active ingredients, the method of formulation, the route of delivery, the specific fusion protein or spore used, the source of the fusion protein or spore (i.e., the spore-producing microbe producing the fusion protein or spore), the target pest species, and the seriousness of the pest infection or damage to the plant(s).

The pest control methods provided herein may be carried out in any suitable manner known to those skilled in the art, depending on the intended aims and prevailing circumstances, that is to say by spraying, wetting, injecting, atomizing, dusting, brushing on, seed dressing, scattering or pouring of a composition comprising a fusion protein or spore described herein. In some embodiments, the composition is applied directly to a plant, plant seed, or plant propagation material. In some embodiments, the composition is applied indirectly to a plant, plant seed, or plant propagation material (e.g., by adding the composition to irrigation water, plant habitat, soil, plant growth container, etc.). In the case of spore forming bacteria and fungi, the application rates with respect to plant propagation material (e.g. seed treatment) may range from about 1×10⁴ to 1×10¹² (or more) spores/seeds. In additional embodiments, the spore concentration may range from about 1×10⁶ to about 1×10¹¹ spores/seed, 1×10⁶ to about 1×10¹⁰ spores/seed, 1×10⁶ to about 1×10⁹ spores/seed, 1×10⁶ to about 1×10⁸ spores/seed, or 1×10⁶ to about 1×10⁷ spores/seed. In additional embodiments, the spore concentration may range from about 1×10⁷ to about 1×10¹¹ spores/seed, 1×10⁷ to about 1×10¹⁰ spores/seed, 1×10⁷ to about 1×10⁹ spores/seed, or 1×10⁷ to about 1×10⁸ spores/seed. In additional embodiments, the spore concentration may be about 1×10⁷ spores/seed.

The propagation material can be treated with the composition prior to use of the propagation material for plant propagation, for example, seed being dressed prior to sowing. The active ingredient may also be applied to seed kernels (coating), either by soaking the kernels in a liquid composition or by coating them with a solid composition. Examples of formulations of compositions comprising a fusion protein or spore described herein that can be used in the methods provided herein include, but are not limited to, solutions, granules, pellets, beads, dusts, sprayable powders, emulsion concentrates, coated granules and suspension concentrates.

### EXAMPLES

The following examples are offered to illustrate, but not to limit the claimed invention.

### Example 1. Cloning Bt cry1Aa and cry1Fa Genes in Spore Display Vectors

Bt Cry insecticidal proteins were displayed on the surface of Bacillus subtilis spores. In order to make the displayed Cry proteins capable of binding to the ABC transporter receptors, a structurally flexible linker including Cry protein's own leader sequences was inserted between spore outer coat (CotC/G) and Cry proteins.

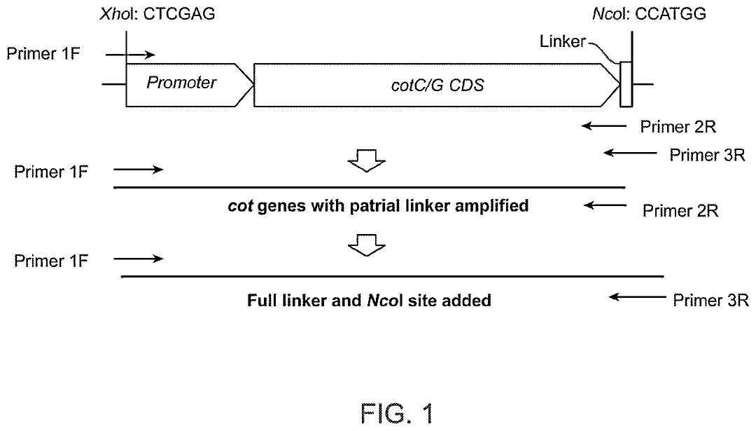

The cry1Aa (SEQ ID NO: 4) and cry1Fa (SEQ ID NO: 2) genes including the leader sequences were synthesized by GenScript Japan (Tokyo, Japan). In order to make Cry1Aa and Cry1Fa resistant to serine protease, R28L or R27L mutation was included in the synthesized cry1Aa or cry1Fa gene. The cotC (SEQ ID NO: 16) and cotG (SEQ ID NO: 18) genes including their promoters (5′ upstream regions) were amplified from a genomic DNA preparation of Bacillus subtilis ISW1214 using Primer 1Fs (SEQ ID NOs: 28 and 29) and Primer 2Rs (SEQ ID NOs: 30 and 31) in Table 2. These primers were designed to link cotC and cotG with nucleotides encoding L1 (short: GGGGSAS [SEQ ID NO:54]) and L2 (long: GGGGSGGGGSAS [SEQ ID NO:55]) linker peptides in the scheme shown in FIG. 1.

As shown in FIG. 1, two-step amplifications were made to obtain promoter-cotC/G-linker (L1 or L2). The first amplification with Primer 3R L1 (SEQ ID NO:32) added a part of the linker, and the second amplification with Primer3R L2 (SEQ ID NO:33) (Table 2) was for the full linker sequence and an NcoI site. The product of the second amplification was cloned in pGEM-T easy (Promega, Madison, WI, USA).

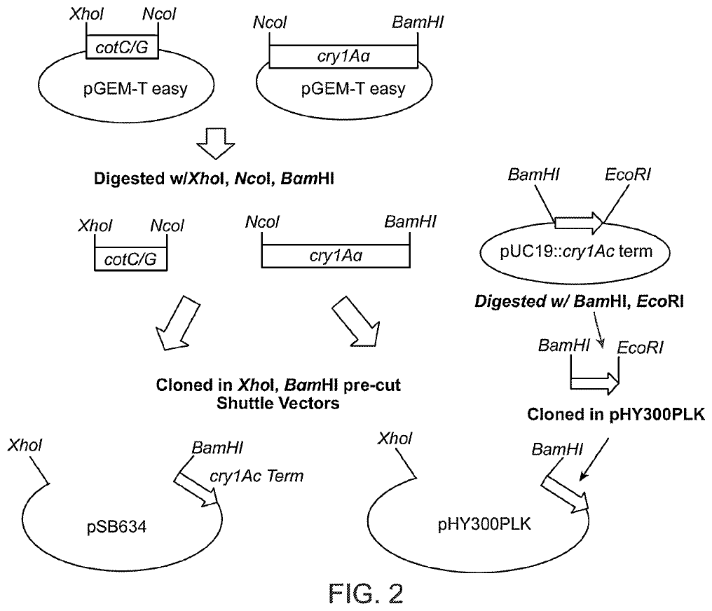

The cry1Aa gene was amplified from the synthetic cry1Aa gene using Primer 4F (SEQ ID NO:34) and Primer 5R (SEq ID NO:35) in Table 2 to add NcoI and BamHI restriction sites. The PCR product with an NcoI site at the 5′ end and BamHI site at the 3′ end of the protein coding sequence was cloned in pGEM-T-easy. These restriction enzyme sites were used to clone other cry genes, such as cry1Fa. These two pGEM-T clones, one containing the cotC/G-L1/L2 genes was digested with XhoI and NcoI, and the other containing cry1Aa was digested with NcoI and BamHI. These two DNA fragments, XhoI-cotC/G-linker-Nco1 and NcoI-cry1Aa-BamHI were cloned by three-way ligation in pSB634 (SEQ ID NO: 22) which had been digested with XhoI and BamHI. This pSB634 plasmid described by Sasaki et al. (1996, Curr. Microbiol. 32: 195-200). In addition, the cot-cry fragment was cloned in pHY300PLK (SEQ ID NO: 23) in which the synthetic cry1Ac-terminator and a XhoI site had been cloned. The cloning scheme is shown in FIG. 2.

Since pHY300PLK does not have any XhoI site in its multiple cloning site (MCS), an XhoI site was inserted between XbaI and BamHI sites in the MCS by cloning a small DNA piece made of Linker F and Linker R oligonucleotides (SEQ ID NOs: 36 and 37) as shown in Table 2.

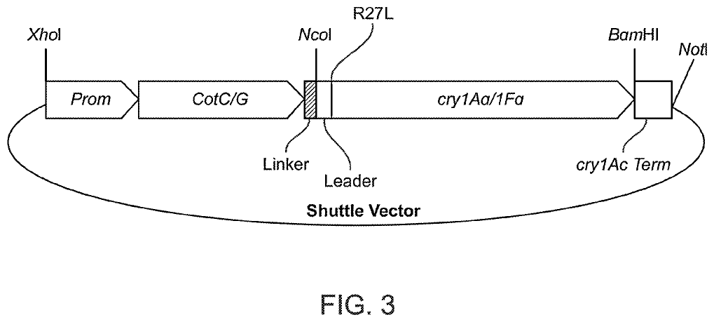

The final spore display plasmids are illustrated in FIG. 3. In this figure, “Shuttle Vector” means pSB634 or pHY300PLK. To clone other cry genes, the cry1Aa gene in the final constructs of pSB634 and pHY300PLK with cotC/G-L1/2-cry1Aa was digested out and a new gene such as cry1Fa was cloned utilizing the NcoI and BamHI sites.

TABLE 2
Primer and linker sequences.

| SEQ | | | |
|---|---|---|---|
| ID | Name | Sequence (5′ to 3′) | Notes |
| 28 | Primer 1F | GTATctcgagAAATCGTTTGGGCCG | Lowercase: XhoI |
| | CotC-F | | restriction enzyme |
| | | | recognition sequence |
| 29 | Primer 1F | GTATctcgagGGATGAACAAACAGC | Lowercase: XhoI |
| | CotG-F | | restriction enzyme |
| | | | recognition sequence |
| 30 | Primer 2R | TGAGCCGCCTCCTCCGTAGTGTT | Bold: partial linker |
| | CotC-R | TTTTATG | |
| 31 | Primer 2R | TGAGCCGCCTCCTCCTTTGTATTT | Bold: partial linker |
| | CotG-R | CTTTTT | |
| 32 | Primer 3R | CATAccatggTGATGCTGAGCCGCC | Bold: full linker; |
| | L1-R | TCCTCC | lowercase: NcoI |
| | | | restriction enzyme |
| | | | recognition sequence |
| 33 | Primer 3R | CATAccatggTGATGCTGATCCTCCT | Bold: full linker; |
| | L2-R | CCTCCTGAGCCGCCTCCTCC | lowercase: NcoI |
| | | | restriction enzyme |
| | | | recognition sequence |
| 34 | Primer 4F | GTATccatggATGGATAACAATCCG | Bold: crylAa coding |
| | | | sequence; lowercase: NcoI |
| | | | restriction enzyme |
| | | | recognition site |
| 35 | Primer 5R | CATAAggatccTTACTTTTGTGCTCT | Bold: cry1Aa coding |
| | | | sequence; lowercase: |
| | | | BamHI restriction enzyme |
| | | | recognition site |
| 36 | Linker F | CTAGACTCGAGAGTCTGACAGctcga | Underline: XbaI and |
| | | gGTCAAGCGTAG | BamHI restriction enzyme |
| | | | recognition sites; |
| | | | lowercase: XhoI |
| | | | restriction enzyme |
| | | | recognition site |
| 37 | Linker R | GATCCTACGCTTGACctcgagCTGTCA | Underline: XbaI and |
| | | GACTCTCGAGT | BamHI restriction enzyme |
| | | | recognition sites; |
| | | | lowercase: XhoI |
| | | | restriction enzyme |
| | | | recognition site |

### Example 2. Preparation of Spore-pSB634, Spore-Cry1Aa and Spore-Cry1Fa

A method of isolating B. subtilis spores expressing a Cry protein on their surface was established. Bacillus cultures were grown on the surface of ½ LB agar plates for full and synchronous sporulation to prevent any contamination of vegetative cells.

After cot-cry genes were cloned in pSB634 and pHY300PLK as shown in FIG. 3, Bacillus subtilis ISW1214 (host strain) was transformed with these plasmid constructs by electroporation. Preparing electrocompetent Bacillus cells and electroporation were done according to a protocol of TaKaRa Bio Inc., Shiga, Japan, from which pHY300PLK and the Bacillus subtilis host strain were purchased. Transformed Bacillus cells were spread on ½ LB-tet agar plates to have confluent growth and incubated at 37° C. After full sporulation, spores were collected with 1 ml TBS (20 mM Tris-HCl, pH 7.5 containing 150 mM NaCl) per plate and transferred in a 15-ml centrifuge tube. The tube was filled with TBS up to 14 ml, and spores were suspended. The spores were centrifuged at 14,000×g for 30 min to save precipitate. The spores in the precipitate was gently but fully suspended in 14-ml TBS without forming air bubbles and centrifuged again at the same speed and time. This procedure of suspending and centrifuging is called “washing,” and this term is used in other Examples meaning the same or similar procedure. The final precipitate was suspended in 1 ml TBS and kept frozen at −20° C. when a long term storage was necessary. This spore suspension was examined under the microscope and the spore count was determined. There was no difference in preparation of Spore-pSB634, except that no cot-cry genes were cloned in pSB634. The wild-type spores of B. subtilis ISW1214 were prepared in the same way except for using 1/2 LB only (no tet) plates.

### Example 3. Checking the Expression of Cry1Aa and Cry1Fa on the Spore Surface

Expression of Bt Cry proteins on the surface of B. subtilis spores was confirmed by microscopic observation and flow cytometry using fluorescent anti-Cry-antibodies. B. subtilis spores expressing a Cry protein could be sorted by flow cytometry.

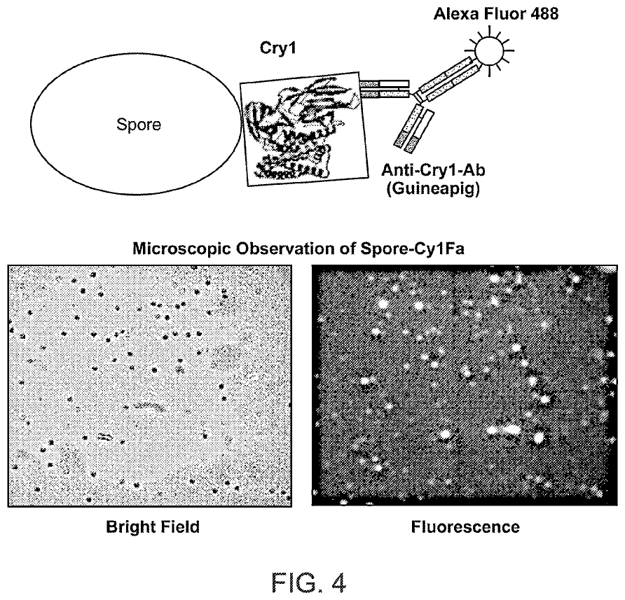

Soluble, spore-free Cry1Aa and Cry1Fa proteins were made by cloning those genes in pSB634 with the cry1Ca promoter as described by Sasaki et al. (1996, Curr. Microbiol. 32:195-200), and Cry proteins were purified according to a method reported by Yamamoto et al. (1989, ACS Symposium Series 432:46-60). Using these purified Cry proteins, antibodies against Cry1Aa and Cry1Fa were made in guinea pig and labeled with Alexa Fluor 488 (“Alexa”) using a labelling kit (Molecular Probe Inc., Eugene, OR, USA). A few μl of 10⁹ spores/ml Spore-Cry1Aa/Cry1Fa suspension in TBS were mixed with an equal volume of the antisera and incubated at 25° C. for 1 hr. Then, the spores were washed in TBS by centrifugation as described in Example 2 and observed under the fluorescence microscope. A model shown on the top panel of FIG. 4 illustrates how the Cry protein on Bacillus spores was detected by the fluorescent antibody. This model shows only one Cry protein molecule on the spore surface, but there are supposed to be multiple molecules. The fluorescent microscopy (FIG. 4, bottom) showed most, if not all, spores were fluorescent confirming that the Cry protein was expressed and localized on the spore surface.

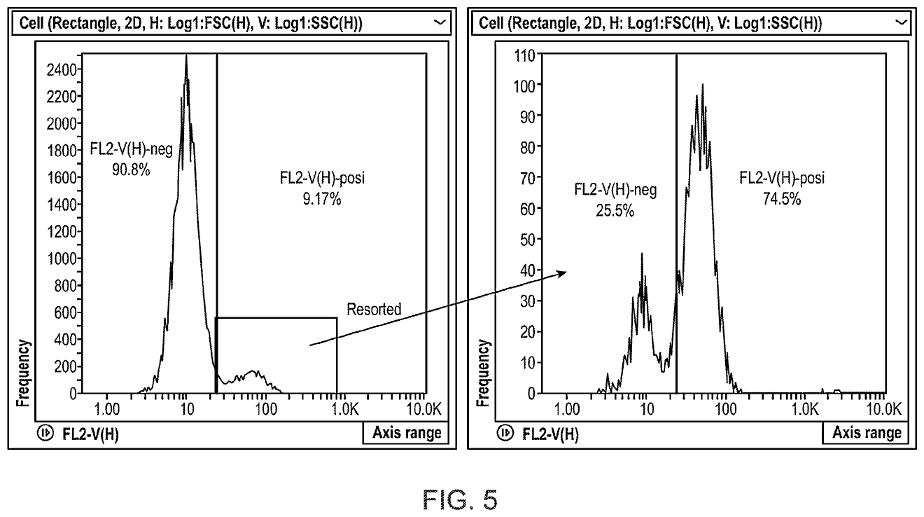

The localization of Cry1Fa on the spore surface was confirmed by flow cytometry using On-chip Sort microchip cell sorter at On-Chip Biotechnologies Co. Ltd., Tokyo, Japan. In this experiment, 10% Spore-Cry1Fa, on which Alexa-labeled anti-Cry1Fa-antibody was bound, were mixed with Spore-Cry1Fa without antibody. As shown in FIG. 5 (left panel), the sorter separated 9.17% fluorescent spores as a small peak. Remaining 90.8% spores (main peak) were not fluorescent. These 9.17% fluorescent spores were resorted (FIG. 5, right panel) and found that their purity was 74.5%. Note the spore count scales were different between two graphs. The result confirmed the microscopic observation that Bacillus spores are indeed expressing the Cry protein. In addition, the result confirmed that Bacillus spores can be sorted by flow cytometry.

### Example 4. Binding Between Sf9 Cells Expressing an ABC Transporter and Bacillus Spores Displaying a Cry Protein

Bt Cry proteins displayed on the surface of B. subtilis spores were capable of binding to their respective receptors which was expressed functionally on the surface of Sf9 insect cells.

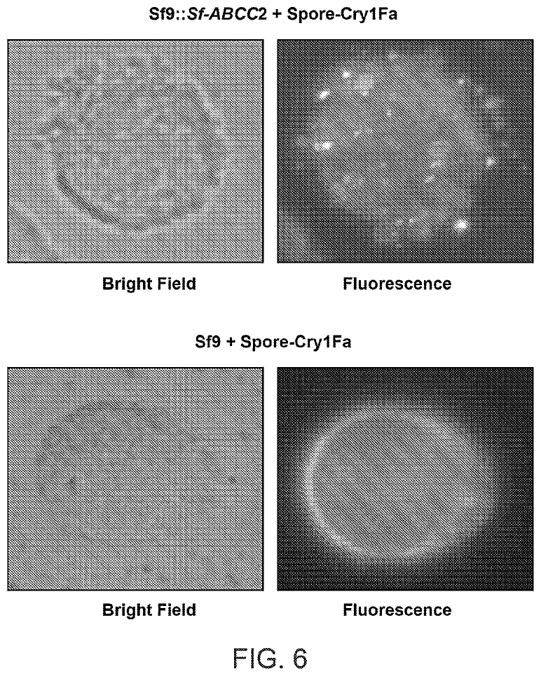

To express ABC transporter proteins in Sf9 cells, genes encoding Bm-ABCC2 (SEQ ID NO: 8), Sf-ABCC2 (SEQ ID NO: 10), Sf-ABCC2-Mutant (SEQ ID NO 12) and Sf-ABCC3 (SEQ ID NO: 14) were synthesized by GenScript Japan, Tokyo, Japan. As shown in these SEQ IDs, these transporters were labeled with a FLAG tag attached to the C-terminus. These genes were cloned in Bac-to-Bac™ Baculovirus Expression System of Thermo-Fisher Scientific, Tokyo, Japan, following their published protocol. Briefly, transporter genes were cloned in pFastBac™ utilizing AcMNPV polyhedrin promoter. EGFP (Enhanced Green Fluorescent Protein) gene, which served as a marker, was also cloned in the same vector between NheI and SphI sites. The GFP gene was amplified from pEGFP-1 (Clontech, Mountain View, CA, USA). After the pFastBac-expression plasmid was transformed into DH10Bac™ cells, transposition occurred between the mini-Tn7 element on the pFastBac vector and the mini-attTn7 target site on the bacmid to generate a recombinant bacmid. After the transposition reaction was completed, the high molecular weight recombinant bacmid DNA was isolated, and the Sf9 cells were transfected with the bacmid DNA using the ExpiFectamine™ Transfection Reagent to generate a recombinant baculovirus. After 72 hours, the media covering the cells was collected and clarified by centrifugation at 500×g for 5 mi. The supernatant was used as a virus solution. Nakaishi et al. (2018, Journal of Insect Biotechnology and Sericology 87(2):45-51), who had cloned Px-ABCC2 with a FLAG tag (SEQ ID NO: 6) in the baculovirus, provided the virus. Sf9 cells expressing this transporter protein was produced by the same way as other transporter genes and used to test the cytotoxicity of Spore-Cry1Aa. Sf9 cells were maintained in Grace's Supplemented Insect Media with 10% heat inactivated fetal bovine serum (Thermo-Fisher) at 26° C. For Spore-Cry binding and cell viability assays, Sf9 cells with >80% confluency were infected with the virus suspension. Infected Sf9 cells were incubated at 26° C. for 72 h. To see if Spore-Cry binds to Sf9 cells expressing ABC transporters, Spore-Cry suspended in PBS (8.1 mM disodium phosphate-1.5 mM monopotassium phosphate buffer, pH 7.4, 2.7 mM potassium chloride, 137 mM NaCl) was mixed with Sf9 cell suspension in insect media and observed under the microscope. In order to confirm that particles seen on the Sf9 cell surface were spores expressing a Cry protein, Alexa-labeled anti-Cry-antibody was added to the spores and observed by fluorescent microscopy. FIG. 6 is an example of Spore-Cry1Fa bound on Sf9 expressing Sf-ABCC2. The bound spores were seen as gray spots on the cell by bright field microscopy (FIG. 6, top left panel) and white spots by fluorescence microscopy (FIG. 6, top right panel). Alexa-labeled anti-Cry1Fa-antibody bound on Cry1Fa on the spores which bound on the Sf9 cell surface. No binding of Spore-Cry1Fa was observed with Sf9 cells without Sf-ABCC2 (FIG. 6, bottom left and bottom right panels) or Sf9 cells expressing Sf-ABCC2-Mutant. The Sf9 cells were visible under the fluorescence microscope, because EGFP was co-cloned with the cry gene.

### Example 5. Cytotoxicity of Bacillus Spores Expressing a Cry Protein

Bt Cry proteins displayed on the surface of Bacillus spores were toxic to the Sf9 insect cells only when the cells were expressing the receptor of the Cry protein.

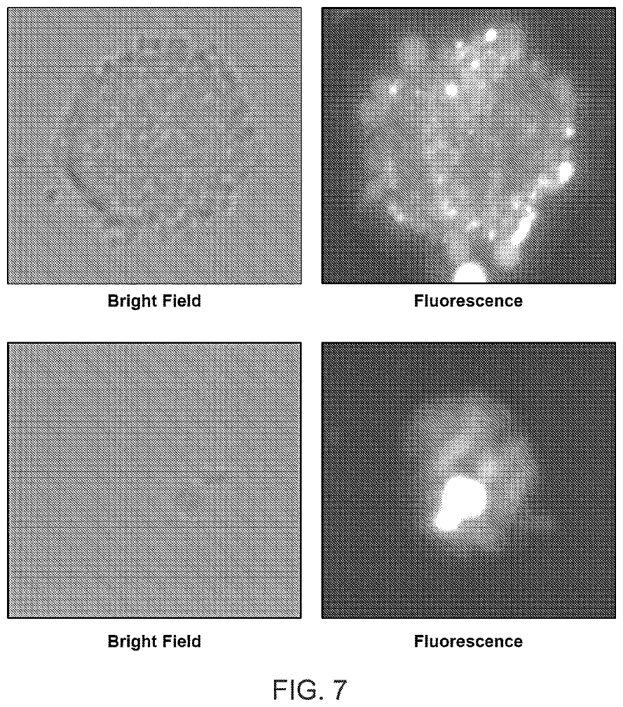

After mixing the cells with Spore-Cry1Fa, the mixture was incubated at 26° C. for 60 min. As shown in FIG. 7, the Sf9 cells expressing Sf-ABCC2 were swollen (FIG. 7, top left and top right panels) and then disrupted completely (FIG. 7, bottom left and bottom right panels). FIG. 7, bottom right panel shows GFP leaked out of the cell indicating the cell wall was completely disintegrated.

The cytotoxicity of Spore-Cry1Fa was not seen with Sf9 cells expressing no ABC transporter nor those expressing Sf-ABCC2-Mutant. Similar observation was seen with the combinations of Spore-Cry1Aa and Sf9 expressing Bm-ABCC2. The cytotoxicity assay of Spore-Cry is summarized as follows: Spore-Cry1Aa was cytotoxic to Px-ABCC2, Bm-ABCC2, Sf-ABCC3; and Spore-Cry1Fa was cytotoxic to Sf-ABCC2 but not to Sf-ABCC2-Mutant from Cry1Fa-resistant S. frugiperda colonies.

### Example 6. Production of ABC Transporter Proteins in Silkworm Pupae

Receptors of Bt Cry insecticidal proteins were expressed in silkworm pupae utilizing a baculovirus expression system to produce a large amount of receptor proteins.

The synthesized Bm-ABCC2, Sf-ABCC2 and Sf-ABCC3 genes were sent to Sysmex Corporation, Kobe, Japan to produce the proteins utilizing their ProCuber™ Technology. Sysmex's ProCube Technology is explained in their website (Sysmex Corporation ProCube Technology, procube.sysmex.co.jp/eng/p/e_silkworm_outline/). Briefly, ABC transporter genes were cloned in a baculovirus expression system to produce viruses. The recombinant baculovirus was propagated in B. mori (silkworm) cells and injected to 10 silkworm pupae. The virus-infected silkworm pupae were homogenized by ultrasonication in TBS containing a protease inhibitor cocktail and a melanization inhibitor. The cell membrane fragments produced by sonication were collected by differential centrifugations as follows. The homogenized pupal tissue was centrifuged at 1,000×g to remove large size materials. The precipitate were further sonicated and centrifuged two more times. The supernatants of the low speed centrifugations were combined and centrifuged at 100,000×g for 1 hr to collect the precipitate. The precipitate was then suspended in 10 ml TBS, divided in ten 1-ml aliquots (1 ml per pupa) and kept frozen at −80° C. This sample, called “Membrane Fragments,” was used to extract and purify the ABC transporter proteins.

### Example 7. Extraction and Purification of ABC Transporter Proteins

ABC transporters produced in silkworm pupae cell membrane were extracted and packaged in SMALP (Styrene Malecic Acid Lipid Particles) and purified by affinity chromatography utilizing a FLAG tag attached to the C-terminus of the second ATPase of the transporter protein. The FLAG tag bound to the anti-FLAG antibody meaning the tag is exposed to the solvent. The result supports the expected structure of ABC transporter proteins in SMALP.

ABC transporter protein was extracted with SMA (Styrene Maleic Acid). In this Example, “solubilized with SMA” or “extracted with SMA” means the ABC transporter protein is extracted from insect cell membrane and packaged in SMALP (Styrene Maleic Acid Lipid Particle) as shown in FIG. 8. Ready-to-use-powder SMA reagents were purchased from Cube Biotech (Monheim, Germany) and dissolved in TBS at 5%. SMA reagents of three different S:M ratios were used to extract Sf-ABCC2. The S:M ratio is styrene (S) to maleic-anhydride (M) ratio in the SMA copolymer. For example S:M=3:1 means a copolymer [Styrene]n-[Maleic-anhydride]m in which n=3 and m=1. The Membrane Fragment suspension prepared in Example 6 was mixed with the equal volume of 5% SMA in TBS and incubated at 25° C. for 2 hr. The mixture was centrifuged at 100,000×g for 30 min at 4° C. The supernatant and precipitate were analyzed by SDS-PAGE and Western blotting using anti-FLAG-antibody to confirm that Sf-ABCC2 in the supernatant. Only SMAs having S:M ratios 2.3:1 and 3:1 extracted Sf-ABCC2, and no Sf-ABCC2 was extracted with SMA of S:M=1.4:1. SMA is supposed to punch out ABC transporter from the cell membrane as a tiny membrane fragment with cell's phospholipid bilayer as shown in FIG. 8. This fragment is in SMALP. In this model (FIG. 8, right panel), ECLs (Extra-Cellular Loops), which bind to the Domain II loop region of Cry protein, and transporter's ATPase regions (nucleotide binding regions) along with the FLAG tag attached to the second ATPase are exposed to the solvent allowing binding to both Cry protein and anti-FLAG-antibody. All ABC transporters used in this invention have a FLAG tag attached at the C-terminus of the second ATPase.

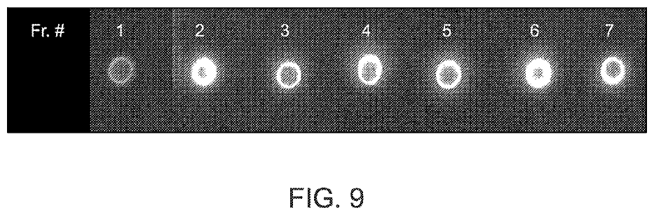

The size of SMALP is determined by the S:M ratio and must be big enough to contain a lipid particle with ABC transporter protein. It seems SMA with S:M ratio 1.4:1 produces lipid particles of an inappropriate size for ABC transporters. SMALP containing Sf-ABCC2 was purified by affinity chromatography using anti-FLAG-antibody-immobilized agarose gel obtained from MBL International Corp., Woburn, MA, USA. One aliquot of 1 ml cell Membrane Fragments sample was divided into two 500 μl aliquots. In one 500 ul aliquot, it was estimated to contain about 150 μg ABC transporter protein by SDS-PAGE. For affinity chromatography, one 500 μl aliquot was mixed with 500 μl 5% SMA, incubated and centrifuged to produce approx. 1 ml SMA-extracted Sf-ABCC2 (i.e., SMALP-Sf-ABCC2). SDS-PAGE indicated that about 50% of the transporter protein were extracted. The SMALP-Sf-ABCC2 was loaded on 200 μl anti-FLAG-antibody-immobilized agarose gel packed in an 1 ml Pierce™ Spin Column (Thermo-Fisher). The column was washed with TBS containing 10% glycerol (TBS-glycerol), and SMALP-Sf-ABCC2 was eluted with 1.2 ml elution buffer (0.1 mg/ml FLAG peptide in TBS—with 10% glycerol). Column eluate was fractionated in six 200 μl fractions and analyzed by dot blotting as shown in FIG. 9. In FIG. 9, all 200 μl fractions (“Fr.”) including the final spin off (Fr. 7) but the first one (Fr. 1) showed strong fluorescent signal confirming Sf-ABCC2 was successfully eluted from the affinity column.

All fractions except for Fr. 1 were combined and concentrated in an Amicon Ultra Centrifugal Filter (50k Da cut off, purchased from MilliporeSigma, Saint Louis, MO, USA) down to 300 ul. The concentrated column eluate was then desalted in a 2 ml Zeba Spin Desalting Column (Thermo-Fisher) to remove FLAG peptide. There was about 300 μl of this affinity purified ABC transporter called SMALP-Sf-ABCC2. SDS-PAGE and Western blotting using anti-FLAG antibody showed one band around 150 kDa. It was divided into six 50 μl aliquots, flash frozen in liquid nitrogen and stored at −80° C. The successful purification of SMALP-Sf-ABCC2 by anti-FLAG-antibody affinity chromatography indicates that the FLAG tag is exposed to the solvent. It supports the SMALP model shown in FIG. 8. It is likely based on the SMALP model that Extra-Cellular Loops (ECLs) of ABCC2 are also exposed to the solvent and are capable of binding to the Cry protein.

### Example 8. Sorting of Bacillus Spores Expressing a Cry Protein Based on Interaction Between the Cry Protein and its Specific Receptor

Bacillus subtilis spores expressing a Cry protein, on which its receptor were bound specifically, were isolated by immuno-magnetic separation using a FLAG tag attached to the receptor and anti-FLAG antibody immobilized on the magnetic beads. Also, it was demonstrated that the spores, which bound to the receptor, could be sorted by flow cytometry.

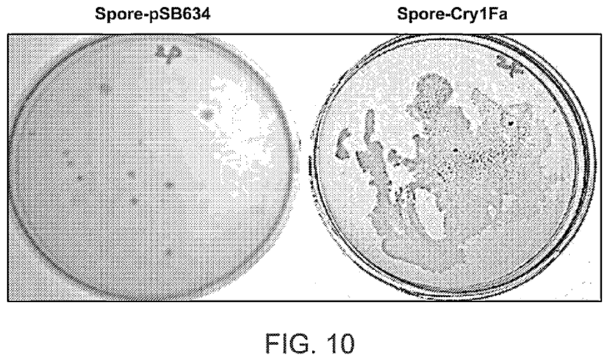

Spore-sorting experiments were done by Immuno-magnetic separation and flow cytometry. The goal of sorting is to pull out Bacillus spores expressing a Cry protein utilizing the affinity between a Cry protein on the spore and a specific ABC transporter protein. To demonstrate the feasibility of spore sorting, Spore-Cry1Fa was sorted with Sf-ABCC2 and Spore-Cry1Aa with Bm-ABCC2. Immuno-magnetic separation was performed with Spore-Cry1Fa, on which SMALP-Sf-ABCC2 bound. The transporter-bound spores were pulled out with anti-FLAG-antibody-immobilized magnetic beads as follows. About 10⁶ spores of Spore-Cry1Fa suspended in 100 μl TBS were mixed with 50 μl affinity-purified SMALP-Sf-ABCC2 from Example 7, incubated for 2 hr at 25° C. and washed twice in 1.5 ml TBS by centrifugation at 14,000×g for 30 min. The washed spores were suspended in 100 μl TBS and mixed with 5 d of anti-FLAG-antibody-immobilized magnetic beads (Cat #M8823 of MilliporeSigma). Spores bound on magnetic beads were held on a magnet and washed with TBS five times. The washed spores were suspended in 100 W TBS. SMALP-Sf-ABCC2 bound Spore-Cry1Fa were detached from anti-FLAG-antibody-immobilized magnetic beads with 1.5 mg/ml FLAG peptide in 10 mM TBS. This final spore suspension was plated on LB-tet plate. The exactly same experiment was done with Spore-pSB634 which has no Cry protein expressed. The results are shown in FIG. 10.

As shown in FIG. 10, the magnetic beads pulled out only 9 spores (colonies) from 10⁶ spores of Spore-pSB634 which have no Cry protein expressed (FIG. 10, left panel). The number of spores was much fewer than expected as a background level of nonspecific binding. On the other hand, the beads captured numerous spores (i.e., too-many-to-count, confluent growth) in the Spore-Cry1Fa starting sample (FIG. 10, right panel).

Similar experiments were done with mixtures of Spore-Cry1Fa and wild-type spores to see if magnetic separation can isolate Spore-Cry1Fa selectively. In Mixture-1, there were 10⁶ (Approx. 10%) Spore-Cry1Fa spores and 10⁷ wild-type spores. Mixture-2 consisted of 2×10³ (0.1%) Spore-Cry1Fa and 2×10⁶ wild-type spores. Mixture-3 contained 2×10³ (0.01%) Spore-Cry1Fa and 2×10⁷ wild-type spores. Mixture-4 had 2×10³ (0.001%) Spore-Cry1Fa and 2×10⁸ wild-type spores. Since these samples were for separating spores between tet-resistant Spore-Cry1Fa and tet-sensitive wild-type spores, the sorted spores could be identified by tet-selection. The spores separated with immuno-magnetic beads were plated on LB plate (no tet) first and then transferred to LB-tet plate. Mixture-1 produced 359 spores (colonies) and Mixture-2 180 spores (colonies), Mixture-3 1500 spores (colonies) and Mixture-4 1700 spores (colonies) on LB-tet plates. In order to increase the recovery of 0.01% and 0.001% Spore-Cry1Fa in Mixure-3 and 4, 50 μl anti-FLAG-antibody-immobilized magnetic beads were used instead of 5 μl. These numbers of spores captured by the magnetic beads indicated that the separation was selective. It was found 70% of spores from Mixture-1, 61% of spores from Miture-2, 60% of spores from Mixture-3 and 70% of spores from Mixture-4 were tetracycline-resistant Spore-Cry1Fa. It strongly indicates that SMALP-Sf-ABCC2 bound selectively to Spore-Cry1Fa confirming the SMALP-ABC transporter model shown in FIG. 8.

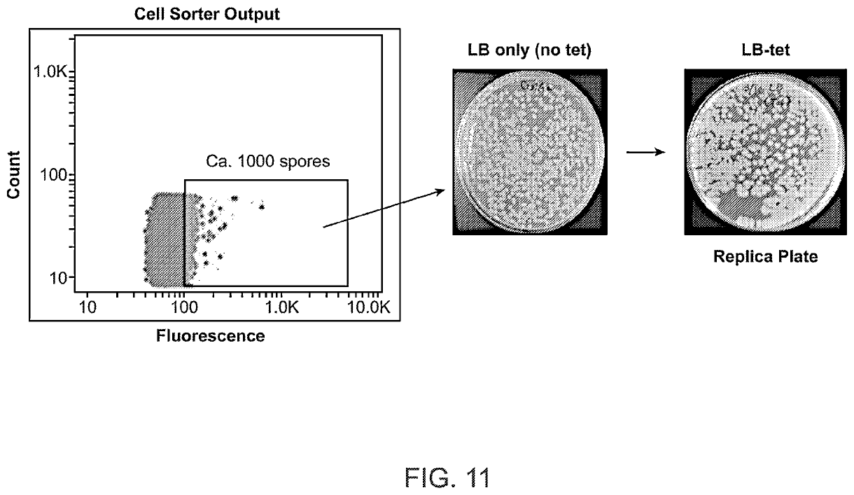

Sorting of Bacillus spores expressing a Cry protein based on the receptor affinity was conducted by flow cytometer. In this case, Spore-Cry1Aa was used along with Bm-ABCC2, which had been solubilized in 1% Triton X100 followed by affinity purification just like SMALP-Sf-ABCC2. Spore-Cry1Aa suspended in PBS was labeled with 5 μl Bm-ABCC2 (approx. 0.3 mg/ml) in PBS containing 1% Triton X100 by simply mixing and incubating at 25° C. for 2 hr. Unbound Bm-ABCCs was removed by washing the spores in 10 mM sodium phosphate buffer, pH 7.4 containing 0.2% SDS (Sodium Dodecyl Sulfate) and 500 mM NaCl. Then, Spore-Cry1Aa-Bm-ABCC2 complex was made fluorescent by mixing with anti-FLAG-mouse-antibody and Alexa-labeled anti-mouse IgG-antibody sequentially. The spore complex was washed in PBS containing 0.02% Triton X100 and observed by fluorescence microscopy. Since the observation revealed only a small number of spores retained the fluorescent dye, those fluorescent spores were sorted by a flow cytometer using On-chip Sort cell sorter (On-Chip Biotechnologies). The cell sorter pulled out about 1000 spores as fluorescent, presumably bound to Bm-ABCC2. Use of 0.2% SDS in the wash solution detached Bm-ABCC2 from many spores, but it was done intentionally to see if the cell sorter could sort out a few remaining fluorescent spores. As shown in FIG. 11, 1000 fluorescent spores were separated and plated on a LB plate. The LB plate was copied on a LB-tet plate. It was found over 800 spores were germinated to form colonies on the tet-selection plate. Some of these colonies were picked, and the plasmid in those spores were confirmed to be the cry1Aa gene by PCR and sequencing (Example 9). This experiment with Spore-Cry1Aa confirmed that Bacillus spores expressing a Cry protein could be sorted by flow cytometry based on the receptor affinity.

### Example 9. Identification of the Cry Gene in the Sorted Spores

In order to identify the Cry protein displayed on the spore surface, the plasmid in the spores sorted by flow cytometry was isolated and sequenced.

To identify the cry gene in the spores sorted by flow cytometry, about 10 colonies on the LB-tet plate shown in FIG. 10 were picked and grown in 1 ml LB-tet broth for a few hours at 37° C. in a shaker until the cell density reached OD_{600 nm}=0.2˜0.3. The cells were collected by centrifugation, and plasmids were isolated from those colonies following a method described by Hou et al. (2019, Toxins 11:162). This plasmid isolation method of Hou et al. is for Bacillus thuringiensis, but it worked well with B. subtilis. The isolated plasmid was sequenced to determine the cry gene in the spore.

All patents, patent publications, patent applications, journal articles, books, technical references, and the like discussed in the instant disclosure are incorporated herein by reference in their entirety for all purposes.

It is to be understood that the figures and descriptions of the disclosure have been simplified to illustrate elements that are relevant for a clear understanding of the disclosure. It should be appreciated that the figures are presented for illustrative purposes and not as construction drawings. Omitted details and modifications or alternative embodiments are within the purview of persons of ordinary skill in the art.

It can be appreciated that, in certain aspects of the disclosure, a single component may be replaced by multiple components, and multiple components may be replaced by a single component, to provide an element or structure or to perform a given function or functions. Except where such substitution would not be operative to practice certain embodiments of the disclosure, such substitution is considered within the scope of the disclosure.

The examples presented herein are intended to illustrate potential and specific implementations of the disclosure. It can be appreciated that the examples are intended primarily for purposes of illustration of the disclosure for those skilled in the art. There may be variations to these diagrams or the operations described herein without departing from the spirit of the disclosure. For instance, in certain cases, method steps or operations may be performed or executed in differing order, or operations may be added, deleted or modified.

Where a range of values is provided, it is understood that each intervening value, to the smallest fraction of the unit of the lower limit, unless the context clearly dictates otherwise, between the upper and lower limits of that range is also specifically disclosed. Any narrower range between any stated values or unstated intervening values in a stated range and any other stated or intervening value in that stated range is encompassed. The upper and lower limits of those smaller ranges may independently be included or excluded in the range, and each range where either, neither, or both limits are included in the smaller ranges is also encompassed within the technology, subject to any specifically excluded limit in the stated range. Where the stated range includes one or both of the limits, ranges excluding either or both of those included limits are also included.

In the foregoing description, numerous specific details are set forth to provide a more thorough understanding of the present invention. However, it will be apparent to one of skill in the art that the invention described in this disclosure may be practiced without one or more of these specific details. In other instances, well-known features and procedures well known to those skilled in the art have not been described in order to avoid obscuring the invention. Embodiments of the disclosure have been described for illustrative and not restrictive purposes. Although the present invention is described primarily with reference to specific embodiments, it is also envisioned that other embodiments will become apparent to those skilled in the art upon reading the present disclosure, and it is intended that such embodiments be contained within the present inventive methods. Accordingly, the present disclosure is not limited to the embodiments described above or depicted in the drawings, and various embodiments and modifications can be made without departing from the scope of the claims below.

INFORMAL SEQUENCE LISTING:

| SEQ | | |
|---|---|---|
| ID | | |
| NO | Name | Sequence |
| 1 | Cry 1Fa | MENNIQNQCVPYNCLNNPEVEILNEELSTG |
| | | RLPLDISLSLTRFLLSEFVPGVGVAFGLFD |
| | | LIWGFITPSDWSLFLLQIEQLIEQRIETLE |
| | | RNRAITTLRGLADSYEIYIEALREWEANPN |
| | | NAQLREDVRIRFANTDDALITAINNFTLTS |
| | | FEIPLLSVYVQAANLHLSLLRDAVSFGQGW |
| | | GLDIATVNNHYNRLINLIHRYTKHCLDTYN |
| | | QGLENLRGTNTRQWARFNQFRRDLTLTVLD |
| | | IVALFPNYDVRTYPIQTSSQLTREIYTSSV |
| | | IEDSPVSANIPNGFNRAEFGVRPPHLMDFM |
| | | NSLFVTAETVRSQTVWGGHLVSSRNTAGNR |
| | | INFPSYGVFNPGGAIWIADEDPRPFYRTLS |
| | | DPVFVRGGFGNPHYVLGLRGVAFQQTGTNH |
| | | TRTFRNSGTIDSLDEIPPQDNSGAPWNDYS |
| | | HVLNHVTFVRWPGEISGSDSWRAPMFSWTH |
| | | RSATPTNTIDPERITQIPLVKAHTLQSGTT |
| | | VVRGPGFTGGDILRRTSGGPFAYTIVNING |
| | | QLPQRYRARIRYASTTNLRIYVTVAGERIF |
| | | AGQFNKTMDTGDPLTFQSFSYATINTAFTF |
| | | PMSQSSFTVGADTFSSGNEVYIDRFELIPV |
| | | TATFEAEYDLERAQK* |
| 2 | Cry 1Fa | ATGGAGAATAATATTCAAAATCAATGCGTA |
| | | CCTTACAATTGTTTAAATAATCCTGAAGTA |
| | | GAAATATTAAATGAAGAATTAAGTACTGGC |
| | | AGATTACCGTTAGATATATCCTTATCGCTT |
| | | ACACGTTTCCTTTTGAGTGAATTTGTTCCA |
| | | GGTGTGGGAGTTGCGTTTGGATTATTTGAT |
| | | TTAATATGGGGTTTTATAACTCCTTCTGAT |
| | | TGGAGCTTATTTCTTTTACAGATTGAACAA |
| | | TTGATTGAGCAAAGAATAGAAACATTGGAA |
| | | AGGAACCGGGCAATTACTACATTACGAGGG |
| | | TTAGCAGATAGCTATGAAATTTATATTGAA |
| | | GCACTAAGAGAGTGGGAAGCAAATCCTAAT |
| | | AATGCACAATTAAGGGAAGATGTGCGTATT |
| | | CGATTTGCTAATACAGACGACGCTTTAATA |
| | | ACAGCAATAAATAATTTTACACTTACAAGT |
| | | TTTGAAATCCCTCTTTTATCGGTCTATGTT |
| | | CAAGCGGCGAATTTACATTTATCACTATTA |
| | | AGAGACGCTGTATCGTTTGGGCAGGGTTGG |
| | | GGACTGGATATAGCTACTGTTAATAATCAT |
| | | TATAATAGATTAATAAATCTTATTCATAGA |
| | | TATACGAAACATTGTTTGGACACATACAAT |
| | | CAAGGATTAGAAAACTTAAGAGGTACTAAT |
| | | ACTCGACAATGGGCAAGATTCAATCAGTTT |
| | | AGGAGAGATTTAACACTTACTGTATTAGAT |
| | | ATCGTTGCTCTTTTTCCGAACTACGATGTT |
| | | AGAACATATCCAATTCAAACGTCATCCCAA |
| | | TTAACAAGGGAAATTTATACAAGTTCAGTA |
| | | ATTGAGGATTCTCCAGTTTCTGCTAATATA |
| | | CCTAATGGTTTTAATAGGGCGGAATTTGGA |
| | | GTTAGACCGCCCCATCTTATGGACTTTATG |
| | | AATTCTTTGTTTGTAACTGCAGAGACTGTT |
| | | AGAAGTCAAACTGTGTGGGGAGGACACTTA |
| | | GTTAGTTCACGAAATACGGCTGGTAACCGT |
| | | ATAAATTTCCCTAGTTACGGGGTCTTCAAT |
| | | CCTGGTGGCGCCATTTGGATTGCAGATGAA |
| | | GATCCACGTCCTTTTTATCGGACATTATCA |
| | | GATCCTGTTTTTGTCCGAGGAGGATTTGGG |
| | | AATCCTCATTATGTACTGGGGCTTAGGGGA |
| | | GTAGCATTTCAACAAACTGGTACGAACCAC |
| | | ACCCGAACATTTAGAAATAGTGGGACCATA |
| | | GATTCTCTAGATGAAATCCCACCTCAGGAT |
| | | AATAGTGGGGCACCTTGGAATGATTATAGT |
| | | CATGTATTAAATCATGTTACATTTGTACGA |
| | | TGGCCAGGTGAGATTTCAGGAAGTGATTCA |
| | | TGGAGAGCTCCAATGTTTTCTTGGACGCAC |
| | | CGTAGTGCAACCCCTACAAATACAATTGAT |
| | | CCGGAGAGGATTACTCAAATACCATTGGTA |
| | | AAAGCACATACACTTCAGTCAGGTACTACT |
| | | GTTGTAAGAGGGCCCGGGTTTACGGGAGGA |
| | | GATATTCTTCGACGAACAAGTGGAGGACCA |
| | | TTTGCTTATACTATTGTTAATATAAATGGG |
| | | CAATTACCCCAAAGGTATCGTGCAAGAATA |
| | | CGCTATGCCTCTACTACAAATCTAAGAATT |
| | | TACGTAACGGTTGCAGGTGAACGGATTTTT |
| | | GCTGGTCAATTTAACAAAACAATGGATACC |
| | | GGTGACCCATTAACATTCCAATCTTTTAGT |
| | | TACGCAACTATTAATACAGCTTTTACATTC |
| | | CCAATGAGCCAGAGTAGTTTCACAGTAGGT |
| | | GCTGATACTTTTAGTTCAGGGAATGAAGTT |
| | | TATATAGACAGATTTGAATTGATTCCAGTT |
| | | ACTGCAACATTTGAAGCAGAATATGATTTA |
| | | GAAAGAGCACAAAAGTAA |
| 3 | Cry 1Aa | MDNNPNINECIPYNCLSNPEVEVLGGELIE |
| | | TGYTPIDISLSLTQFLLSEFVPGAGFVLGL |
| | | VDIIWGIFGPSQWDAFPVQIEQLINQRIEE |
| | | FARNQAISRLEGLSNLYQIYAESFREWEAD |
| | | PTNPALREEMRIQFNDMNSALTTAIPLLAV |
| | | QNYQVPLLSVYVQAANLHLSVLRDVSVFGQ |
| | | RWGFDAATINSRYNDLTRLIGNYTDYAVRW |
| | | YNTGLERVWGPDSRDWVRYNQFRRELTLTV |
| | | LDIVALFSNYDSRRYPIRTVSQLTREIYTN |
| | | PVLENFDGSFRGMAQRIEQNIRQPHLMDIL |
| | | NSITIYTDVHRGFNYWSGHQITASPVGFSG |
| | | PEFAFPLFGNAGNAAPPVLVSLTGLGIFRT |
| | | LSSPLYRRIILGSGPNNQELFVLDGTEFSF |
| | | ASLTTNLPSTIYRQRGTVDSLDVIPPQDNS |
| | | VPPRAGFSHRLSHVTMLSQAAGAVYTLRAP |
| | | TFSWQHRSAEFNNIIPSSQITQIPLTKSTN |
| | | LGSGTSVVKGPGFTGGDILRRTSPGQISTL |
| | | RVNITAPLSQRYRVRIRYASTTNLQFHTSI |
| | | DGRPINQGNFSATMSSGSNLQSGSFRTVGF |
| | | TTPFNFSNGSSVFTLSAHVFNSGNEVYIDR |
| | | IEFVPAEVTFEAEYDLERAQK* |
| 4 | Cry 1Aa | ATGGATAACAATCCGAACATCAATGAATGC |
| | | ATTCCTTATAATTGTTTAAGTAACCCTGAA |
| | | GTAGAAGTATTAGGTGGAGAATTAATAGAA |
| | | ACTGGTTACACCCCAATCGATATTTCCTTG |
| | | TCGCTAACGCAATTTCTTTTGAGTGAATTT |
| | | GTTCCCGGTGCTGGATTTGTGTTAGGACTA |
| | | GTTGATATAATATGGGGAATTTTTGGTCCC |
| | | TCTCAATGGGACGCATTTCTTGTACAAATT |
| | | GAACAGTTAATTAACCAAAGAATAGAAGAA |
| | | TTCGCTAGGAACCAAGCCATTTCTAGATTA |
| | | GAAGGACTAAGCAATCTTTATCAAATTTAC |
| | | GCAGAATCTTTTAGAGAGTGGGAAGCAGAT |
| | | CCTACTAATCCAGCATTAAGAGAAGAGATG |
| | | CGTATTCAATTCAATGACATGAACAGTGCC |
| | | CTTACAACCGCTATTCCTCTTTTGGCAGTT |
| | | CAAAATTATCAAGTTCCTCTTTTATCAGTA |
| | | TATGTTCAAGCTGCAAATTTACATTTATCA |
| | | GTTTTGAGAGATGTTTCAGTGTTTGGACAA |
| | | AGGTGGGGATTTGATGCCGCGACTATCAAT |
| | | AGTCGTTATAATGATTTAACTAGGCTTATT |
| | | GGCAACTATACAGATTATGCTGTGCGCTGG |
| | | TACAATACGGGATTAGAGCGTGTATGGGGA |
| | | CCGGATTCTAGAGATTGGGTAAGGTATAAT |
| | | CAATTTAGAAGAGAGCTAACACTTACTGTA |
| | | TTAGATATCGTTGCTCTATTCTCAAATTAT |
| | | GATAGTCGAAGGTATCCAATTCGAACAGTT |
| | | TCCCAATTAACAAGAGAAATTTATACGAAC |
| | | CCAGTATTAGAAAATTTTGATGGTAGTTTT |
| | | CGTGGAATGGCTCAGAGAATAGAACAGAAT |
| | | ATTAGGCAACCACATCTTATGGATATCCTT |
| | | AATAGTATAACCATTTATACTGATGTGCAT |
| | | AGA |
| 5 | Px-ABCC2 | GGCTTTAATTATTGGTCAGGGCATCAAATA |
| | | ACAGCTTCTCCTGTAGGGTTTTCAGGACCA |
| | | GAATTCGCATTCCCTTTATTTGGGAATGCG |
| | | GGGAATGCAGCTCCACCCGTACTTGTCTCA |
| | | TTAACTGGTTTGGGGATTTTTAGAACATTA |
| | | TCTTCACCTTTATATAGAAGAATTATACTT |
| | | GGTTCAGGCCCAAATAATCAGGAACTGTTT |
| | | GTCCTTGATGGAACGGAGTTTTCTTTTGCC |
| | | TCCCTAACGACCAACTTGCCTTCCACTATA |
| | | TATAGACAAAGGGGTACAGTCGATTCACTA |
| | | GATGTAATACCGCCACAGGATAATAGTGTA |
| | | CCACCTCGTGCGGGATTTAGCCATCGATTG |
| | | AGTCATGTTACAATGCTGAGCCAAGCAGCT |
| | | GGAGCAGTTTACACCTTGAGAGCTCCAACG |
| | | TTTTCTTGGCAGCATCGCAGTGCTGAATTT |
| | | AATAATATAATTCCTTCATCACAAATTACA |
| | | CAAATACCTTTAACAAAATCTACTAATCTT |
| | | GGCTCTGGAACTTCTGTCGTTAAAGGACCA |
| | | GGATTTACAGGAGGAGATATTCTTCGAAGA |
| | | ACTTCACCTGGCCAGATTTCAACCTTAAGA |
| | | GTAAATATTACTGCACCATTATCACAAAGA |
| | | TATCGGGTAAGAATTCGCTACGCTTCTACT |
| | | ACAAATTTACAATTCCATACATCAATTGAC |
| | | GGAAGACCTATTAATCAGGGTAATTTTTCA |
| | | GCAACTATGAGTAGTGGGAGTAATTTACAG |
| | | TCCGGAAGCTTTAGGACTGTAGGTTTTACT |
| | | ACTCCGTTTAACTTTTCAAATGGATCAAGT |
| | | GTATTTACGTTAAGTGCTCATGTCTTCAAT |
| | | TCAGGCAATGAAGTTTATATAGATCGAATT |
| | | GAATTTGTTCCGGCAGAAGTAACCTTTGAG |
| | | GCAGAATATGATTTAGAAAGAGCACAAAAG |
| | | TAAMENGSGARKESEEKKEVKKGKPNVLSR |
| | | LFMCWVCPVLVGGNRRDVEERDLIPPPSAK |
| | | YKSESLGDKFERYWLEELGLATQRGVAPSL |
| | | WRALRRAFWLSYMPGALLLLGNAIPRTIQP |
| | | LLFTRLLSYWSADSTMTRLEAGYWAMGMLL |
| | | CNFLAMVCHHHNTLFVGRFGMKVRIACCSL |
| | | LYRKLLRLNQRSLQSTAAGKLVNLMSNDVA |
| | | RFDYAFMFLHYFWMIPLQSAAVLYFMFRAA |
| | | GWAPIVGLFSVMLLILPIQAGLTKLTAVYR |
| | | RETAQRTDKRIKLMSEIINGIQVIKMYAWE |
| | | IPFQKVVGSSRAHEVEALKRASFVQGTFGG |
| | | FMLFTERTSLFLTVMTLVLTGSMATATTVY |
| | | PIQQYFSIIQSNLALILPIAIAQLTEMLVS |
| | | LERLQEFLMLDEREDLSVMPGGQADTAPVA |
| | | FKYTKETTAPAYIVSKRYSKKEDDTGLAAE |
| | | LVERKSTSEFAVELNDVSASWGGEGDKDQH |
| | | TLRGVSLRVRRGKLAAIIGPVGSGKSSLLQ |
| | | VLLKELPVSSGTVGVHGQISYACQESWLFS |
| | | ATVRDNILFGLPYDSKKYKKVCDACCLQPD |
| | | FKQFPYGDLSLVGERGVSLSGGQRARINLA |
| | | RAVYRDADIYIFDDPLSAVDANVGRQLFEG |
| | | CINGYLRGRTRVLVTHQIHFLKAADYIVIL |
| | | NEGAIENMGTYDDLTKLENSLLLPKQQEGS |
| | | GDDSKDELAIPNAANKPIMERGVSVISVKS |
| | | EDNGEARKEQVQAAEERASGNLKWEVFARY |
| | | LVSVDSWAIVALTLTAMLITQGAASSTDYW |
| | | LSFWTNQVDGYIQDLPDGEEPDPSLGTQTG |
| | | ILLTGQYVYIYGALVLTIIVMSFMRLFGFV |
| | | TMTMRAAANIHDLMFRNLIRATMRFFDTNP |
| | | SGRVLNRFSKDMGGMDELLPRSILQAFQMY |
| | | LSMASVLTLNAVSLPWTLIPTVLLLGLFIR |
| | | YLKWYLNAAQSVKRLEGTTKSPVFGMIGST |
| | | LSGMSTIRSSDSQDRLIKNFDDCQNLHTSA |
| | | FHTYIGGATAFGFYLDMICLVYLASILSIF |
| | | ILIDFADVIPVGSVGLAVSQSMVLTVLLQL |
| | | AARFTSDFLAQMTAVERVLEYTKLPHEENI |
| | | NDGPTQPPKTWPAEGNIKFENVFLTYSLED |
| | | PPVLKNINFEIQSGWKVGVVGRTGAGKSSL |
| | | ISALFRLTNLDGSIKIDGIDTIGIAKQELR |
| | | AKISIIPQEPVLFSATLRYNLDPFDLYSDD |
| | | DIWRALEQVELKDVVPALDYKVSEGGSNFS |
| | | VGQRQLLCLARAVLRSNKILVMDEATANVD |
| | | PQTDALIQSTIRRQFAACTVLTIAHRLNTV |
| | | MDSDRVLVMDKGEVVEFDHPYTLLSAPGSH |
| | | LNFMVEETGDNMSKALYDMAKKKYFDDHPQ |
| | | DYKDDDDK |
| 6 | Px-ABCC2 | ATGGAAAACGGAAGCGGAGCCCGGAAAGAG |
| | | TCGGAAGAGAAGAAAGAGGTGAAGAAAGGC |
| | | AAGCCCAACGTGCTGTCCCGGCTGTTCATG |
| | | TGCTGGGTGTGCCCCGTGCTGGTGGGCGGC |
| | | AACCGGCGCGACGTGGAGGAGCGGGACCTC |
| | | ATACCGCCGCCCAGCGCCAAGTACAAGTCC |
| | | GAGTCTCTCGGAGACAAGTTTGAAAGGTAC |
| | | TGGCTAGAAGAGCTGGGTCTGGCGACGCAG |
| | | CGCGGGGTGGCGCCGTCGTTGTGGCGTGCG |
| | | CTGCGCCGCGCCTTCTGGCTGTCCTACATG |
| | | CCGGGCGCGCTGCTGCTGCTCGGGAACGCC |
| | | ATACCCAGGACCATCCAGCCGCTGCTATTC |
| | | ACGCGACTGCTCTCCTATTGGTCGGCGGAC |
| | | AGCACCATGACCCGACTGGAGGCCGGCTAT |
| | | TGGGCCATGGGCATGCTGCTGTGCAACTTC |
| | | CTGGCCATGGTGTGCCACCACCACAACACG |
| | | CTATTCGTCGGCCGCTTCGGCATGAAAGTC |
| | | AGGATTGCTTGCTGCTCGCTGCTCTATCGG |
| | | AAGCTGCTCCGTCTAAACCAGAGGTCCCTG |
| | | CAAAGCACGGCAGCGGGCAAGCTGGTGAAC |
| | | CTGATGTCGAACGACGTGGCGCGCTTCGAC |
| | | TACGCCTTCATGTTCCTGCACTACTTCTGG |
| | | ATGATCCCGCTGCAGTCGGCCGCCGTGCTG |
| | | TACTTCATGTTCCGCGCCGCCGGCTGGGCG |
| | | CCCATCGTGGGCCTGTTCTCTGTCATGCTG |
| | | CTCATACTGCCTATACAAGCCGGCCTCACC |
| | | AAGCTCACAGCTGTCTACAGGAGGGAGACT |
| | | GCACAAAGGACAGATAAGAGAATTAAACTA |
| | | ATGAGCGAAATTATCAATGGTATTCAGGTG |
| | | ATAAAGATGTACGCGTGGGAGATCCCCTTC |
| | | CAGAAGGTGGTGGGTTCCTCCCGCGCGCAC |
| | | GAGGTGGAGGCGTTGAAGCGGGCGTCCTTC |
| | | GTGCAGGGCACCTTCGGGGGGTTCATGCTG |
| | | TTCACGGAGCGCACCTCGCTCTTCCTCACC |
| | | GTCATGACGCTCGTGCTGACTGGGAGCATG |
| | | GCTACTGCTACTACGGTGTATCCAATCCAA |
| | | CAATACTTTAGTATAATCCAATCCAATTTG |
| | | GCGCTCATCCTCCCTATCGCCATCGCTCAA |
| | | CTCACTGAGATGTTGGTCTCATTGGAAAGA |
| | | CTTCAGGAGTTTCTGATGTTAGACGAGAGG |
| | | GAAGACCTGTCGGTGATGCCGGGCGGGCAG |
| | | GCGGACACCGCGCCCGTGGCCTTCAAGTAC |
| | | ACGAAGGAGACCACGGCGCCCGCCTACATC |
| | | GTGTCCAAGAGGTACTCCAAGAAGGAGGAC |
| | | GACACCGGCCTGGCTGCGGAGCTGGTGGAG |
| | | CGCAAATCCACGAGCGAGTTCGCGGTCGAG |
| | | CTGAACGACGTGAGTGCGTCGTGGGGGGGT |
| | | GAGGGGGACAAGGACCAGCACACGCTGCGG |
| | | GGGGTGAGCCTGCGCGTGCGCCGCGGCAAG |
| | | CTGGCCGCCATCATCGGACCCGTCGGCTCG |
| | | GGGAAGTCGTCGCTACTCCAAGTGTTGCTG |
| | | AAGGAGCTTCCGGTGTCTTCCGGCACCGTG |
| | | GGTGTGCACGGCCAGATCTCGTACGCGTGT |
| | | CAGGAGTCCTGGCTCTTCTCCGCCACGGTT |
| | | CGGGATAACATCCTCTTCGGACTGCCGTAT |
| | | GATTCGAAGAAATATAAGAAGGTGTGCGAC |
| | | GCGTGCTGCCTGCAGCCCGACTTCAAGCAG |
| | | TTCCCGTACGGAGACCTGTCGCTGGTGGGC |
| | | GAGCGCGGCGTGTCGCTGTCCGGGGGGCAG |
| | | CGCGCCCGGATCAACCTCGCGCGCGCCGTG |
| | | TACCGGGACGCCGACATCTACATATTCGAT |
| | | GACCCCTTATCGGCGGTGGACGCGAATGTG |
| | | GGTCGGCAGCTGTTCGAGGGCTGCATCAAC |
| | | GGCTACCTGCGCGGCCGCACGCGCGTGCTC |
| | | GTCACGCATCAGATACACTTCCTCAAGGCC |
| | | GCAGACTACATAGTCATACTCAACGAGGGT |
| | | GCCATTGAAAATATGGGCACGTACGATGAT |
| | | TTAACGAAGCTGGAGAATTCGCTGCTGCTG |
| | | CCCAAGCAACAGGAGGGTTCCGGCGATGAC |
| | | AGTAAGGATGAACTAGCTATTCCGAATGCA |
| | | GCAAACAAGCCGATAATGGAACGGGGGGTA |
| | | TCAGTGATCTCAGTGAAGAGCGAAGACAAC |
| | | GGTGAGGCTCGCAAGGAACAGGTGCAGGCG |
| | | GCGGAGGAGCGCGCCTCCGGGAACCTCAAG |
| | | TGGGAGGTGTTCGCGAGGTACCTGGTCTCC |
| | | GTGGACTCCTGGGCCATCGTGGCGCTCACG |
| | | CTCACCGCGATGCTCATCACGCAGGGGGCG |
| | | GCGTCGTCCACCGACTACTGGCTTAGCTTC |
| | | TGGACCAATCAAGTTGATGGATACATACAA |
| | | GACCTGCCAGATGGGGAGGAGCCAGATCCA |
| | | AGTCTCGGCACGCAAACGGGCATCCTGCTG |
| | | ACGGGCCAGTACGTGTACATCTACGGCGCG |
| | | CTGGTGCTCACCATCATCGTGATGTCCTTC |
| | | ATGCGGCTGTTCGGCTTCGTGACCATGACC |
| | | ATGCGCGCCGCCGCCAACATCCACGACCTC |
| | | ATGTTCCGAAACCTCATCCGCGCCACCATG |
| | | CGCTTCTTTGACACCAATCCTTCAGGTCGC |
| | | GTGCTAAACCGGTTCTCCAAAGACATGGGC |
| | | GGCATGGACGAGCTGCTGCCCAGGTCCATC |
| | | CTGCAGGCCTTCCAGATGTACCTGTCCATG |
| | | GCCAGCGTGCTCACGCTGAACGCCGTCTCC |
| | | CTGCCCTGGACCCTCATACCCACGGTGCTG |
| | | CTGCTGGGACTCTTCATCAGGTACCTCAAG |
| | | TGGTACCTGAACGCTGCGCAGTCTGTGAAG |
| | | AGGCTGGAGGGCACAACCAAGAGTCCAGTG |
| | | TTTGGAATGATCGGCTCTACTCTATCGGGA |
| | | ATGTCGACAATCAGAAGTTCAGACTCACAG |
| | | GACAGACTAATCAAAAACTTCGACGACTGC |
| | | CAGAATCTCCATACTTCTGCCTTCCACACT |
| | | TACATCGGGGGAGCCACGGCCTTCGGATTC |
| | | TATTTGGATATGATATGCCTCGTCTACCTC |
| | | GCGTCTATATTATCAATTTTCATCCTTATC |
| | | GACTTCGCGGACGTGATCCCGGTGGGCAGC |
| | | GTGGGGCTGGCGGTGAGCCAGTCCATGGTG |
| | | CTGACGGTGCTGCTGCAGCTGGCGGCGCGG |
| | | TTCACCAGCGACTTCCTGGCGCAGATGACC |
| | | GCCGTGGAGCGCGTGCTCGAGTACACTAAG |
| | | CTGCCGCACGAGGAGAATATTAATGATGGC |
| | | CCTACGCAGCCGCCCAAGACATGGCCTGCT |
| | | GAAGGGAACATCAAATTTGAAAACGTCTTC |
| | | CTGACTTACTCGTTGGAGGACCCACCCGTG |
| | | CTCAAAAATATTAACTTCGAGATCCAAAGC |
| | | GGTTGGAAGGTGGGCGTGGTCGGCAGGACC |
| | | GGGGCCGGGAAGTCATCTCTCATATCTGCC |
| | | CTTTTCCGATTGACCAATTTAGACGGAAGC |
| | | ATTAAAATTGATGGGATCGATACAATAGGA |
| | | ATAGCTAAGCAGGAGCTCAGGGCGAAGATT |
| | | TCAATTATACCGCAAGAGCCTGTTCTGTTC |
| | | TCGGCCACGCTGCGCTACAACCTGGACCCC |
| | | TTCGACTTGTACAGTGACGACGATATTTGG |
| | | AGAGCTTTGGAACAGGTGGAGTTGAAAGAC |
| | | GTGGTTCCGGCGCTCGACTACAAAGTGTCG |
| | | GAAGGAGGTAGCAATTTCTCCGTGGGACAG |
| | | CGGCAGCTGCTGTGCCTGGCGCGCGCCGTG |
| | | CTGCGCTCCAACAAGATACTAGTCATGGAC |
| | | GAGGCCACCGCCAATGTCGACCCGCAGACG |
| | | GACGCGCTGATCCAGTCGACGATCCGGCGG |
| | | CAGTTCGCGGCCTGCACCGTGCTCACCATC |
| | | GCGCATCGTCTCAACACCGTCATGGACTCC |
| | | GACCGAGTGCTCGTCATGGACAAGGGTGAA |
| | | GTGGTCGAGTTCGACCACCCGTACACGCTG |
| | | CTGTCAGCCCCCGGCAGCCACCTCAACTTC |
| | | ATGGTGGAGGAGACCGGAGACAACATGAGC |
| | | AAGGCCCTTTATGACATGGCCAAGAAGAAA |
| | | TACTTCGACGACCATCCTCAAGACTACAAA |
| | | GACGATGACGACAAGTAG |
| 7 | Bm-ABCC2 | MNSDGRAGENSSAETRRKPHKPNILSRIFL |
| | | WWMCPVLVKGNQRDTVEDDLIIPKKSFNSE |
| | | NQGEYLERYWLQEYEAAIKEKREPSLWTAL |
| | | RKAYWLGYMPGAIYLIIISVFRIIQPLVFA |
| | | ELLSYWSVEATITRLEASYYALALLGINFI |
| | | NMMCQHHNSLFVARFGLKVKVACSSLVYRK |
| | | LLRMDQVALGDVSGGKLVNLLSNDVARFDY |
| | | AFMFLHYLWVVPVQAAVVLYFLYISAGYAP |
| | | FVGFFGVVILILPIQAGLTKLTSVVRRETA |
| | | QRTDRRIKLMTEIINGIQVIKMYAWEKPFQ |
| | | AIVKVARNFEMIALRKSIFIRSVFLGFMLF |
| | | TERSIIFITCLTFLLTGNLVTATLIYPIQQ |
| | | YISIIQINLTMILPLAIASLSEMLVSLERI |
| | | QNFLVKDEREDVQITPKSYGDDNRLIFNNK |
| | | ASLGPQNEIIPKKYLATDGQLASTLTNEPV |
| | | LSTDPAVCDYPIELSKVDATWSSSTDTSEM |
| | | TLRNITLRIGRGKLCAIIGPVGSGKSSILQ |
| | | VLLKELPVCGGSLRINGRLSYACQESWLFP |
| | | ATVRENILFGLPYDSQKYHEVCKACSLLPD |
| | | FKQFPYGDLSLVGERGVSLSGGQRARINLA |
| | | RAVYREADIYLLDDPLSAVDANVGRQLFDG |
| | | CIKGYLRGRTCVLVTHQIHYLKAADIIVIL |
| | | NEGAIENVGSYDDLVNTGTEFSKLLTNQES |
| | | NDNENGPEKNFLRAISKISTKSVEDPDNEK |
| | | VQVEEEEKRAKGNLKFSVLYKYLSAVKSWF |
| | | LVFLMVVTLVITQGCATFIDYWLSFWTNQV |
| | | DEYEQSLAEGEEPSTSLDTQAGAFTLGVYL |
| | | WTYGGVILILIVISHVRILTFVITTMRASS |
| | | NFHDTVYKKLIITVMRFFDMNPSGRVLNRF |
| | | SKDMGAMDEFLPRSLFETVQMYLTLCSILI |
| | | LNAIALPWTLIPTAVLLILFFVLLKWYLNA |
| | | AQAVKRLEGTTKSPVLGMINSTLTGLSTIR |
| | | SSNSQGRLLQMFDNAQNLHTSAFYTFVGGS |
| | | TAFGLYLDALCLVYLGVILTIFLVIDFSTL |
| | | IPVGSVGLAVSQSMVLTMMLQMAARFTADF |
| | | LGQMTAVERVLEYTELPMEENMYDGSQLPK |
| | | DWPTHGRIEFQNLFLNYSQEDPPVLKDLNF |
| | | VIENGWKVGVVGRTGAGKSSMISALFRLYE |
| | | LQGHIRIDGIDTNIIAKTELRSKISIIPQE |
| | | PILFSASVRYNLDPFDSYSDDEIWRALEQV |
| | | ELKEVIPALDYKVSEGGSNFSVGQRQLVCL |
| | | ARAVLRSNKILVMDEATANVDPQTDALIQT |
| | | TIRREFASCTVITIAHRLNTIMDSDRVLVM |
| | | DKGVVAEYDTPYALLSDPNSIFSSMVRETG |
| | | DTMSKVLFRVAEDKHLGRNTEKDYKDDDDK |
| 8 | Bm-ABCC2 | ATGAATAGTGATGGGAGAGCCGGAGAAAAT |
| | | TCTAGTGCTGAGACGCGAAGGAAGCCCCAT |
| | | AAGCCGAACATTTTGTCTCGTATATTTCTA |
| | | TGGTGGATGTGCCCCGTACTCGTTAAAGGA |
| | | AATCAAAGAGACACTGTCGAGGATGACCTC |
| | | ATAATTCCGAAAAAGTCATTTAATTCTGAA |
| | | AATCAAGGAGAGTATTTAGAAAGGTATTGG |
| | | CTCCAAGAATATGAAGCCGCGATAAAGGAG |
| | | AAAAGGGAGCCGTCATTGTGGACGGCTTTA |
| | | CGAAAAGCTTACTGGTTGGGATACATGCCG |
| | | GGAGCTATCTACTTGATAATCATATCTGTC |
| | | TTCAGGATAATTCAGCCGCTAGTATTCGCC |
| | | GAACTGCTTTCCTACTGGTCAGTAGAAGCA |
| | | ACTATAACTCGACTAGAAGCAAGCTATTAT |
| | | GCTCTAGCACTTTTAGGTATAAACTTCATT |
| | | AATATGATGTGTCAACATCACAATTCATTG |
| | | TTTGTAGCGCGCTTTGGACTCAAGGTTAAA |
| | | GTGGCTTGCTCATCTTTAGTATATAGAAAG |
| | | TTGTTGCGCATGGACCAAGTAGCCTTAGGA |
| | | GATGTAAGCGGAGGGAAACTGGTGAACCTG |
| | | TTATCTAATGATGTTGCTAGATTCGACTAC |
| | | GCATTCATGTTTTTGCACTACCTCTGGGTG |
| | | GTACCAGTTCAAGCGGCTGTAGTACTATAT |
| | | TTCTTATATATTTCTGCGGGATACGCACCG |
| | | TTCGTCGGCTTCTTTGGGGTTGTGATTTTA |
| | | ATTCTACCTATACAAGCTGGTCTCACAAAA |
| | | TTGACCTCTGTGGTCAGAAGAGAAACTGCT |
| | | CAGAGAACCGACCGAAGAATAAAATTGATG |
| | | ACGGAAATTATTAATGGCATTCAGGTTATT |
| | | AAAATGTACGCATGGGAGAAACCGTTCCAA |
| | | GCAATCGTCAAGGTGGCAAGAAACTTCGAA |
| | | ATGATAGCACTCAGGAAGTCTATATTCATT |
| | | CGGAGTGTTTTCCTTGGTTTCATGCTGTTC |
| | | ACTGAAAGAAGTATAATATTTATAACATGT |
| | | TTGACATTTTTACTTACGGGTAACCTTGTT |
| | | ACAGCTACTCTGATTTACCCTATTCAACAA |
| | | TACATCAGCATTATTCAAATCAATTTGACT |
| | | ATGATACTGCCTCTTGCCATTGCAAGTTTG |
| | | TCTGAAATGCTGGTCTCGTTGGAACGCATT |
| | | CAAAATTTCTTGGTGAAAGATGAAAGAGAG |
| | | GACGTCCAAATAACACCAAAATCTTATGGA |
| | | GATGATAATAGATTGATTTTCAACAACAAG |
| | | GCATCGCTTGGCCCTCAAAACGAAATTATC |
| | | CCAAAGAAATATTTAGCCACCGATGGCCAA |
| | | CTTGCCTCAACATTAACGAATGAACCAGTG |
| | | CTTTCAACTGACCCAGCCGTGTGTGACTAT |
| | | CCCATAGAATTATCTAAAGTAGACGCCACA |
| | | TGGTCGTCATCAACAGATACTTCAGAAATG |
| | | ACGCTAAGGAATATTACTCTCAGAATAGGC |
| | | AGAGGCAAACTTTGTGCTATAATTGGACCT |
| | | GTTGGATCAGGAAAGTCATCCATTTTGCAA |
| | | GTTCTTCTGAAAGAACTGCCGGTTTGCGGT |
| | | GGTAGCCTGAGAATAAATGGGAGGCTTTCC |
| | | TACGCGTGTCAAGAGTCTTGGCTGTTCCCT |
| | | GCTACTGTGAGAGAAAACATTTTGTTTGGT |
| | | TTGCCGTACGACTCACAGAAATATCACGAG |
| | | GTGTGTAAAGCTTGTTCGTTGCTTCCTGAT |
| | | TTCAAACAATTTCCGTACGGAGACCTCTCC |
| | | TTAGTTGGAGAGCGAGGAGTATCCTTGTCA |
| | | GGGGGACAGCGTGCCAGAATTAATCTCGCT |
| | | AGGGCTGTTTACCGAGAGGCCGACATTTAC |
| | | TTGCTAGATGATCCTCTCTCAGCAGTGGAT |
| | | GCCAACGTTGGCAGGCAGCTGTTTGACGGT |
| | | TGTATCAAAGGATATCTGCGGGGTCGAACT |
| | | TGTGTCCTTGTAACCCATCAAATCCACTAT |
| | | CTTAAAGCAGCTGACATTATTGTCATACTG |
| | | AACGAGGGAGCAATTGAAAACGTTGGTAGC |
| | | TACGATGATTTGGTCAACACCGGAACAGAG |
| | | TTTTCGAAGCTGTTGACTAATCAAGAAAGC |
| | | AACGATAATGAGAATGGTCCGGAAAAGAAT |
| | | TTCTTACGGGCTATATCGAAAATATCTACT |
| | | AAGAGTGTTGAAGATCCAGATAACGAGAAG |
| | | GTACAAGTGGAGGAAGAAGAGAAACGTGCC |
| | | AAAGGAAATTTGAAATTCTCTGTTTTATAC |
| | | AAGTATTTGTCTGCAGTGAAATCATGGTTT |
| | | CTAGTATTTCTGATGGTAGTCACATTAGTT |
| | | ATCACACAAGGATGTGCGACGTTCATAGAT |
| | | TATTGGCTGAGTTTTTGGACAAATCAAGTT |
| | | GATGAGTATGAACAATCTTTAGCTGAGGGA |
| | | GAAGAACCGAGCACAAGTTTGGACACGCAA |
| | | GCCGGAGCGTTCACTTTAGGAGTATACCTT |
| | | TGGACTTATGGCGGAGTCATCTTAATCCTC |
| | | ATAGTGATCAGTCACGTCAGAATTTTAACT |
| | | TTCGTCATTACGACTATGAGAGCTTCATCT |
| | | AATTTTCATGATACCGTTTATAAAAAGTTG |
| | | ATTATCACAGTTATGCGATTCTTTGACATG |
| | | AATCCCTCAGGTCGTGTGTTGAATCGTTTT |
| | | TCAAAGGATATGGGTGCGATGGACGAATTC |
| | | CTTCCGAGGAGTTTATTTGAAACTGTTCAG |
| | | ATGTATTTAACATTATGCAGTATACTTATT |
| | | TTAAATGCTATAGCATTACCGTGGACGTTG |
| | | ATCCCAACAGCGGTACTTTTGATTCTCTTC |
| | | TTCGTTCTCCTGAAATGGTACTTAAATGCT |
| | | GCTCAAGCTGTAAAAAGATTAGAAGGCACA |
| | | ACCAAGAGTCCAGTACTGGGCATGATCAAC |
| | | TCAACCCTCACAGGTCTGTCTACGATCCGA |
| | | AGTTCTAACTCACAAGGACGACTGCTACAA |
| | | ATGTTTGATAACGCACAGAATCTCCACACG |
| | | AGTGCATTTTATACGTTTGTTGGTGGATCA |
| | | ACTGCATTTGGTTTATATCTGGATGCGCTT |
| | | TGCCTAGTTTATTTGGGAGTGATATTGACG |
| | | ATCTTCCTTGTAATCGACTTCAGTACTTTA |
| | | ATTCCCGTCGGTAGCGTGGGCTTGGCTGTG |
| | | AGTCAGTCTATGGTGTTAACGATGATGCTG |
| | | CAAATGGCGGCTCGGTTCACAGCCGATTTC |
| | | CTCGGACAAATGACGGCTGTCGAGAGAGTT |
| | | CTCGAATATACCGAGCTTCCGATGGAAGAA |
| | | AACATGTATGATGGATCCCAACTTCCCAAG |
| | | GACTGGCCTACACACGGTCGTATTGAATTT |
| | | CAAAATCTATTTTTGAACTATTCTCAAGAA |
| | | GATCCACCAGTACTCAAAGATTTAAACTTT |
| | | GTAATTGAGAATGGTTGGAAGGTTGGAGTA |
| | | GTCGGTCGAACCGGTGCCGGCAAGTCTTCT |
| | | ATGATATCCGCACTGTTCAGGCTGTACGAA |
| | | CTGCAAGGACACATCAGAATAGACGGAATC |
| | | GATACCAACATCATCGCTAAAACGGAGCTA |
| | | AGATCGAAGATTTCTATCATTCCACAAGAA |
| | | CCGATTTTGTTCTCTGCATCGGTGCGATAC |
| | | AATTTGGATCCCTTCGACAGTTATAGCGAC |
| | | GACGAAATTTGGAGAGCTTTGGAACAGGTT |
| | | GAACTGAAAGAGGTAATTCCAGCGCTCGAT |
| | | TATAAAGTGTCAGAAGGTGGCAGCAACTTC |
| | | TCCGTCGGTCAGCGACAACTGGTATGTTTG |
| | | GCTCGAGCTGTTTTGCGTTCTAACAAAATC |
| | | CTTGTAATGGACGAGGCGACAGCCAACGTT |
| | | GATCCTCAAACTGACGCGTTGATTCAGACG |
| | | ACGATACGTCGCGAGTTTGCGTCATGTACG |
| | | GTGATCACGATCGCGCACCGACTCAACACG |
| | | ATCATGGATTCTGATCGCGTCCTGGTCATG |
| | | GACAAAGGAGTTGTTGCCGAATACGATACT |
| | | CCTTATGCTCTGCTCTCCGACCCCAATTCG |
| | | ATCTTCAGCTCGATGGTGCGCGAAACAGGA |
| | | GATACAATGAGCAAAGTCCTCTTCCGAGTG |
| | | GCTGAAGATAAACATCTTGGTAGAAATACA |
| | | GAAAAAGACTACAAAGACGATGACGACAAG |
| | | TAG |
| 9 | Sf-ABCC2 | MDKSNKNTAANGNGGQRAGEPKKRVRKKPN |
| | | ILSRIFVWWIFPVLITGNKRDVEEDDLIVP |
| | | SKKFNSERQGEYFERYWFEEVAIAEREDRD |
| | | PSLWKAMRRAYWLQYMPGAIFVLLISGLRT |
| | | AQPLLFSQLLSYWSVDSEMSQQDAGLYALA |
| | | MLGINFITMMCTHHNNLFVMRFSMKVKIAA |
| | | SSLLFRKLLRMSQVSVGDVAGGKLVNLLSN |
| | | DVARFDYAFMFLHYLWVVPLQVGVVLYFVY |
| | | DAAGWAPYVGLFGVIILIMPLQAGLTKLTG |
| | | VVRRMTAKRTDKRIKLMSEIINGIQVIKMY |
| | | AWEKPFQLVVKAARAYEMSALRKSIFIRSM |
| | | FLGFMLFTERSVMFLTVLTLALTGNMISAT |
| | | LIYPIQQYFGIITMNVTLILPMAFASFSEM |
| | | LISLERIQGFLLLDERSDIQITPKVVNGAG |
| | | SKLFNNSKKEGGLETGIVLPTKYSPTEASI |
| | | ARPVQDEPNMADYPVQLNKVNATWADLNDN |
| | | KEMTLKNISLRVRKNKLCAVIGPVGSGKTS |
| | | LLQLLLRELPVTSGNLSISGTVSYASQEPW |
| | | LFPATVRENILFGLEYNVAKYKEVCKVCSL |
| | | LPDFKQFPYGDLSLVGERGVSLSGGQRARI |
| | | NLARAVYREADIYLLDDPLSAVDANVGRQL |
| | | FDGCIKGYLSGKTCILVTHQIHYLKAADFI |
| | | VVLNEGSVENMGSYDELMKTGTEFSMLLSD |
| | | QASEGSDTDKKERPAMMQGISKMSVKSDDE |
| | | EGEEKVQVLEAEERQSGSLKWDVLGRYMKS |
| | | VNSWCMVVMAFLVLVITQGAATTTDYWLSF |
| | | WTNQVDGYIQTLPEGESPNPELDTQVGLLT |
| | | TGQYLIVHGSVVLAIIILTQVRILSFVVMT |
| | | MRASENLHNTIYEKLIVAVMRFFDTNPSGR |
| | | VLNRFSKDMGAMDELLPRSMLETVQMYLSL |
| | | ASVLVLNAIALPWTLIPTTVLMFIFVFLLK |
| | | WYINAAQAVKRLEGTTKSPVFGMINSTISG |
| | | LSTIRSSNSQDRLLNSFDDAQNLHTSAFYT |
| | | FLGGSTAFGLYLDTLCLIYLGIIMSIFILG |
| | | DFGELIPVGSVGLAVSQSMVLTMMLQMAAK |
| | | FTADFLGQMTAVERVLEYTKLPTEENMETG |
| | | PTTPPKGWPSAGEVTFSNVYLKYSPDDPPV |
| | | LKDLNFAIKSGWKVGVVGRTGAGKSSLISA |
| | | LFRLSDITGSIKIDGLDTQGIAKKLLRSKI |
| | | SIIPQEPVLFSASLRYNLDPFDNYNDEDIW |
| | | RALEQVELKESIPALDYKVSEGGTNFSMGQ |
| | | RQLVCLARAILRSNKILIMDEATANVDPQT |
| | | DALIQKTIRKQFANCTVLTIAHRLNTIMDS |
| | | DRVLVMDQGVAAEFDHPYILLSNPNSKFSS |
| | | MVKETGDNMSRILFEVAKTKYESDSKTADY |
| | | KDDDDK |
| 10 | Sf-ABCC2 | ATGGACAAATCTAATAAAAATACCGCGGCA |
| | | AATGGCAATGGTGGTCAACGTGCGGGCGAA |
| | | CCAAAGAAGAGAGTAAGAAAAAAACCAAAC |
| | | ATATTGTCACGTATATTTGTTTGGTGGATA |
| | | TTTCCTGTGCTCATTACTGGTAACAAGCGA |
| | | GACGTAGAAGAAGACGATTTAATTGTTCCA |
| | | AGTAAAAAATTTAACTCTGAAAGACAAGGA |
| | | GAATATTTTGAAAGATATTGGTTCGAGGAG |
| | | GTAGCAATTGCTGAGAGAGAAGACAGAGAT |
| | | CCTTCACTATGGAAAGCGATGCGGCGCGCT |
| | | TACTGGCTGCAATACATGCCGGGAGCTATC |
| | | TTCGTGCTGCTCATTTCTGGATTAAGGACA |
| | | GCTCAGCCATTGCTGTTCTCTCAGCTCCTG |
| | | TCCTACTGGTCGGTGGACAGTGAGATGTCG |
| | | CAGCAGGATGCTGGTCTCTATGCCCTGGCC |
| | | ATGTTGGGGATTAACTTTATCACCATGATG |
| | | TGCACGCACCACAACAACTTGTTCGTCATG |
| | | CGATTCAGTATGAAAGTCAAAATCGCCGCC |
| | | TCGTCACTCTTATTTAGAAAGTTGCTCCGC |
| | | ATGAGTCAAGTGTCAGTGGGCGATGTCGCC |
| | | GGTGGAAAGTTAGTGAACTTGCTGTCCAAC |
| | | GATGTCGCGAGGTTTGACTATGCGTTCATG |
| | | TTCCTACACTACCTGTGGGTGGTACCGCTG |
| | | CAAGTCGGAGTTGTCCTGTACTTCGTGTAC |
| | | GATGCTGCTGGGTGGGCGCCATATGTCGGA |
| | | CTCTTTGGAGTGATTATATTAATCATGCCA |
| | | CTTCAAGCCGGTCTAACTAAACTCACGGGT |
| | | GTTGTGAGACGAATGACAGCTAAGAGGACC |
| | | GACAAAAGAATTAAGCTTATGAGTGAAATT |
| | | ATCAACGGTATACAGGTTATCAAAATGTAC |
| | | GCTTGGGAGAAACCCTTCCAGCTAGTGGTG |
| | | AAGGCGGCTCGTGCGTATGAAATGAGTGCT |
| | | CTTAGGAAATCTATCTTCATCAGGAGCATG |
| | | TTCCTCGGGTTTATGTTGTTCACCGAGCGA |
| | | AGTGTCATGTTTCTGACTGTGCTGACTCTA |
| | | GCGCTGACTGGAAACATGATTAGTGCCACT |
| | | CTGATTTATCCCATCCAACAGTACTTCGGT |
| | | ATTATCACAATGAACGTTACCCTTATTTTA |
| | | CCGATGGCGTTTGCAAGTTTCTCTGAGATG |
| | | TTGATATCGTTGGAACGTATTCAAGGATTC |
| | | CTACTTTTGGACGAGCGTTCAGACATTCAA |
| | | ATTACACCAAAAGTGGTGAATGGTGCTGGA |
| | | AGCAAATTGTTCAACAATTCCAAGAAGGAG |
| | | GGAGGCCTCGAGACTGGCATTGTGTTGCCA |
| | | ACAAAATACTCACCTACCGAAGCAAGTATT |
| | | GCCAGACCCGTGCAGGATGAGCCTAACATG |
| | | GCCGACTATCCGGTGCAACTTAACAAAGTG |
| | | AATGCAACATGGGCGGACCTCAATGATAAC |
| | | AAAGAAATGACACTCAAGAATATATCTTTA |
| | | CGTGTTCGCAAAAACAAACTATGCGCTGTT |
| | | ATTGGACCTGTGGGATCAGGAAAGACCTCT |
| | | CTTCTTCAGCTCCTTTTGAGGGAGTTGCCC |
| | | GTGACTAGCGGCAACCTCAGCATATCTGGT |
| | | ACCGTATCCTATGCTAGTCAGGAGCCTTGG |
| | | CTGTTCCCTGCGACCGTGCGGGAGAACATT |
| | | CTGTTTGGTTTGGAATACAATGTCGCCAAA |
| | | TATAAAGAGGTTTGTAAAGTCTGCTCATTA |
| | | CTGCCAGACTTTAAGCAGTTCCCGTACGGT |
| | | GACTTGTCTCTGGTTGGGGAGCGAGGTGTG |
| | | TCACTGTCTGGTGGTCAGAGGGCCAGGATC |
| | | AATTTGGCCAGAGCTGTCTACCGCGAGGCT |
| | | GATATTTACTTGCTTGACGATCCTCTATCG |
| | | GCTGTAGACGCAAACGTCGGCAGGCAACTA |
| | | TTCGACGGCTGCATCAAAGGATACCTCAGT |
| | | GGCAAAACATGCATCCTGGTCACCCACCAA |
| | | ATTCACTACCTTAAAGCTGCAGACTTTATT |
| | | GTAGTCCTAAACGAGGGTTCCGTCGAAAAT |
| | | ATGGGCTCGTATGATGAACTAATGAAAACT |
| | | GGAACGGAATTCTCGATGCTGCTCTCTGAC |
| | | CAAGCTAGTGAAGGCTCTGACACTGACAAA |
| | | AAAGAACGGCCAGCAATGATGCAAGGAATA |
| | | TCAAAGATGTCAGTCAAGAGTGACGATGAG |
| | | GAAGGTGAGGAGAAGGTTCAAGTATTGGAG |
| | | GCCGAAGAGAGACAGTCGGGCAGCCTGAAG |
| | | TGGGATGTGCTTGGGAGGTACATGAAGTCG |
| | | GTCAACTCCTGGTGCATGGTGGTAATGGCA |
| | | TTCCTCGTGTTGGTGATCACGCAGGGTGCT |
| | | GCCACCACTACCGACTACTGGCTTAGTTTC |
| | | TGGACTAATCAAGTGGATGGATACATACAA |
| | | ACTTTACCCGAAGGAGAAAGCCCAAATCCT |
| | | GAGTTGGATACTCAAGTCGGTCTATTAACC |
| | | ACGGGACAGTACCTCATTGTGCACGGCAGC |
| | | GTAGTACTAGCCATTATAATATTGACGCAA |
| | | GTCAGAATACTTTCCTTCGTAGTGATGACT |
| | | ATGCGAGCTTCGGAAAATCTTCATAACACC |
| | | ATTTACGAGAAATTGATAGTGGCTGTAATG |
| | | AGATTTTTCGATACCAATCCATCGGGTCGT |
| | | GTATTGAACAGATTCTCAAAAGATATGGGT |
| | | GCTATGGATGAGCTGTTGCCGCGAAGCATG |
| | | TTGGAGACTGTTCAGATGTACCTGTCTCTA |
| | | GCCAGTGTCCTTGTGCTGAACGCCATAGCC |
| | | TTACCTTGGACATTGATACCCACCACAGTG |
| | | TTGATGTTTATATTTGTATTCCTATTGAAG |
| | | TGGTACATTAACGCTGCTCAAGCTGTGAAA |
| | | CGATTGGAAGGAACTACAAAGAGTCCAGTG |
| | | TTTGGAATGATCAACTCTACTATATCTGGA |
| | | CTCTCCACCATAAGAAGCTCCAACTCTCAG |
| | | GACCGACTTCTAAACTCATTTGATGATGCA |
| | | CAGAATCTCCATACTAGCGCTTTCTACACG |
| | | TTTTTGGGTGGTTCAACCGCATTTGGTCTG |
| | | TACCTGGACACCCTCTGTTTAATCTACCTC |
| | | GGAATTATCATGTCCATCTTCATTCTTGGT |
| | | GATTTCGGTGAGCTGATACCGGTTGGTAGC |
| | | GTCGGTCTGGCCGTCAGTCAGTCCATGGTG |
| | | CTCACCATGATGTTGCAAATGGCCGCCAAG |
| | | TTCACTGCCGACTTCCTGGGACAGATGACA |
| | | GCCGTCGAGAGGGTCCTGGAGTACACCAAG |
| | | CTACCTACCGAAGAAAACATGGAGACTGGA |
| | | CCGACAACGCCACCAAAGGGCTGGCCAAGT |
| | | GCTGGAGAGGTGACGTTCTCAAACGTGTAC |
| | | CTCAAGTATTCTCCTGACGACCCACCAGTA |
| | | CTGAAGGACTTGAACTTTGCAATCAAGAGT |
| | | GGATGGAAGGTCGGAGTAGTTGGTAGAACT |
| | | GGTGCTGGCAAGTCCTCGTTGATATCAGCT |
| | | CTGTTCCGGCTCAGCGACATTACAGGCAGT |
| | | ATCAAAATTGATGGCCTGGATACTCAAGGA |
| | | ATTGCCAAGAAGCTTTTGAGATCAAAAATA |
| | | TCAATAATTCCGCAAGAGCCAGTGTTGTTC |
| | | TCTGCCTCGCTGCGTTACAATCTGGACCCA |
| | | TTCGACAACTACAACGATGAAGATATTTGG |
| | | AGAGCATTGGAACAGGTGGAACTAAAGGAG |
| | | AGTATACCAGCCCTCGATTACAAAGTGTCG |
| | | GAAGGCGGCACCAACTTCTCAATGGGACAA |
| | | CGTCAGCTGGTGTGCCTGGCGCGCGCCATC |
| | | CTCCGCTCAAATAAAATTCTCATCATGGAC |
| | | GAAGCCACCGCTAACGTCGATCCTCAGACG |
| | | GATGCTTTGATCCAGAAAACAATTCGTAAA |
| | | CAATTTGCCAACTGCACCGTGCTCACGATC |
| | | GCGCATCGACTGAACACCATTATGGATTCA |
| | | GATCGGGTACTAGTCATGGACCAGGGAGTG |
| | | GCCGCGGAGTTCGACCACCCCTACATCTTG |
| | | CTGTCCAACCCCAACAGCAAGTTCTCCTCA |
| | | ATGGTGAAGGAAACAGGCGACAACATGTCC |
| | | AGAATCTTGTTCGAAGTAGCCAAAACAAAA |
| | | TATGAAAGTGATTCCAAAACCGCTGACTAC |
| | | AAAGACGATGACGACAAGTAG |
| 11 | Sf-ABCC2-Mut | MDKSNKNTAANGNGGQRAGEPKERVRKKPN |
| | | ILSRIFVWWIFPVLITGNKRDVEEDDLIVP |
| | | SKKFNSERQGEYFERYWFEEVAIAEREDRD |
| | | PSLWKAMRRAYWLQYMPGAIFVLLISGLRT |
| | | AQPLLFSQLLSYWSVDSEMSQQDAGLYALA |
| | | MLGINFITMMCTHHNNLFVMRFSMKVKIAA |
| | | SSLLFRKLLRMSQVSVGDVAGGKLVNLLSN |
| | | DVARFDYAFMFLHYLWVVPLQVGVVLYFVY |
| | | DAAGWAPYVGLFGVIILIMPLQAGLTKLTG |
| | | VVRRMTAKRTDKRIKLMSEIINGIQVIKMY |
| | | AWEKPFQLVVKAARAYEMSALRKSIFIRSM |
| | | FLGFMLFTERSVMFLTVLTLALTGNMISAT |
| | | LIYPIQQYFGIITMNVTLILPMAFASFSEM |
| | | LISLERIQGFLLLDERSDIQITPKVVNGAG |
| | | SKLENNSKKEGGLETGIVLPTKYSPTEANM |
| | | ARPMQDEPNMADYPVQLNKVNATWADLNDN |
| | | KEMTLKNISSRVRKNKLCAVIGPVGSGKTS |
| | | LLQLLLRELPVTSGNLSISGTVSYASQEPW |
| | | LFPATVRENILFGLEYNVAKYKEVCKVCSL |
| | | LPDFKQFPYGDLSLVGERGVSLSGGQRARI |
| | | NLARAVYREADIYLLDDPLSAVDANVGRQL |
| | | FDGCIKGYLSGKTCILVTHQIHYLKAADFI |
| | | VVLNEGSVENMGSYDELMKTGTEFSMLLSD |
| | | QASEGSDTDKKERPAMMRGISKMSVKSDDE |
| | | EGEEKVQVLEAEERQSGSLKWDVLGRYMKS |
| | | VNSWCMVVMAFLVLVITQGAATTTDYWLSF |
| | | WTNQVDIQTLPEGESKNPELNTQVGLLTTG |
| | | QYLIVHGSVVLAIIILTQVRILSFVVMTMR |
| | | ASENLHNTIYEKLIVAVMRFFDTNPSGRVL |
| | | NRFSKDMGAMDELLPRSMLETVQMYLSLAS |
| | | VLVLNAIALPWTLIPTTVLMFIFVFLLKWY |
| | | INAAQAVKRLEGTTKSPVFGMINSTISGLS |
| | | TIRSSNSQDRLLNSFDDAQNLHTSAFYTFL |
| | | GGSTAFGLYLDTLCLIYLGIIMSIFILGDF |
| | | GELIPVGSVGLAVSQSMVLTMMLQMAAKFT |
| | | ADFLGQMTAVERVLEYTKLPTEENMETGPT |
| | | TPPKDWPSAGEVTFSNVYLKYSPDDPPVLK |
| | | DLNFAIKSGWKVGVVGRTGAGKSSLISALF |
| | | RLSDITGSIKIDGLDTQGIAKKLLRSKISI |
| | | IPQEPVLFSASLRYNLDPFDNYNDEDIWRA |
| | | LEQVELKESIPALDYKVSEGGTNFSMGQRQ |
| | | LVCLARAILRSNKILIMDEATANVDPQTDA |
| | | LIQKTIRKQFATCTVLTIAHRLNTIMDSDR |
| | | VLVMDQGVAAEFDHPYILLSNPNSKFSSMV |
| | | KETGDNMSRILFEVAKTKYESDSKTADYKD |
| | | DDDK |
| 12 | Sf-ABCC2-Mut | ATGGACAAATCTAATAAAAATACCGCGGCA |
| | | AATGGCAATGGTGGTCAACGTGCGGGCGAA |
| | | CCAAAGGAGAGAGTAAGAAAAAAACCAAAC |
| | | ATATTATCACGTATATTTGTTTGGTGGATA |
| | | TTCCCTGTGCTCATCACTGGCAACAAGCGA |
| | | GATGTAGAAGAAGACGATCTAATTGTTCCG |
| | | AGTAAAAAATTCAATTCTGAAAGACAAGGA |
| | | GAATATTTTGAAAGATATTGGTTCGAGGAG |
| | | GTAGCAATTGCTGAGAGAGAAGACCGAGAT |
| | | CCTTCGTTATGGAAAGCGATGCGCCGCGCT |
| | | TACTGGCTGCAATACATGCCGGGAGCTATC |
| | | TTCGTGCTGCTCATTTCTGGATTAAGGACA |
| | | GCTCAGCCATTGCTGTTCTCTCAGCTCCTG |
| | | TCCTACTGGTCGGTGGACAGCGAGATGTCG |
| | | CAGCAGGATGCTGGTCTCTATGCCCTGGCC |
| | | ATGCTGGGGATCAACTTCATCACCATGATG |
| | | TGCACGCACCACAACAACTTGTTCGTTATG |
| | | CGGTTCAGTATGAAAGTCAAAATCGCCGCC |
| | | TCGTCACTCTTATTTAGAAAGTTGCTCCGC |
| | | ATGAGTCAAGTGTCAGTGGGCGATGTCGCT |
| | | GGTGGAAAGTTAGTGAACTTGCTGTCTAAC |
| | | GATGTCGCGAGGTTTGACTACGCGTTCATG |
| | | TTCCTGCACTACCTGTGGGTGGTACCGCTG |
| | | CAAGTCGGAGTCGTCCTGTACTTCGTGTAC |
| | | GATGCTGCTGGGTGGGCGCCATATGTCGGT |
| | | CTCTTTGGAGTCATCATCTTAATCATGCCA |
| | | CTTCAAGCCGGTCTAACTAAACTCACGGGT |
| | | GTTGTGAGACGGATGACAGCTAAGAGGACT |
| | | GACAAAAGAATTAAGCTTATGAGTGAAATT |
| | | ATCAACGGTATACAGGTCATCAAAATGTAC |
| | | GCTTGGGAGAAACCCTTCCAGCTAGTGGTG |
| | | AAGGCGGCTCGTGCGTATGAAATGAGTGCT |
| | | CTTAGGAAGTCTATCTTCATCAGGAGCATG |
| | | TTCCTCGGGTTTATGTTGTTCACCGAGCGA |
| | | AGTGTCATGTTTCTGACTGTGCTGACTCTA |
| | | GCGTTGACTGGAAATATGATTAGTGCCACT |
| | | CTGATTTATCCCATCCAACAGTACTTCGGT |
| | | ATTATCACAATGAACGTTACCCTCATTTTA |
| | | CCGATGGCGTTTGCAAGTTTCTCTGAGATG |
| | | TTGATATCCTTGGAACGTATTCAGGGATTC |
| | | CTTCTTTTGGACGAGCGTTCAGACATTCAA |
| | | ATTACACCAAAAGTGGTGAATGGTGCTGGA |
| | | AGTAAATTGTTCAACAATTCCAAGAAGGAG |
| | | GGAGGCCTCGAGACTGGCATTGTGCTGCCA |
| | | ACAAAATACTCACCTACTGAAGCGAATATG |
| | | GCCAGACCCATGCAGGATGAGCCTAACATG |
| | | GCTGACTATCCTGTGCAACTTAACAAAGTT |
| | | AATGCAACATGGGCGGACCTCAATGATAAC |
| | | AAAGAGATGACACTTAAGAATATATCTTCA |
| | | CGTGTTCGCAAAAACAAACTATGCGCTGTT |
| | | ATTGGACCTGTGGGATCAGGAAAGACCTCT |
| | | CTTCTTCAGCTCCTTTTGAGAGAATTGCCT |
| | | GTAACTAGTGGTAACCTCAGCATATCTGGT |
| | | ACCGTATCCTACGCTAGTCAGGAGCCGTGG |
| | | CTGTTCCCTGCGACCGTGCGGGAGAACATT |
| | | CTGTTTGGTTTGGAATACAATGTCGCCAAA |
| | | TATAAAGAGGTTTGTAAAGTCTGCTCATTA |
| | | CTGCCAGACTTTAAGCAGTTCCCGTACGGT |
| | | GACTTGTCTCTGGTTGGAGAACGAGGTGTG |
| | | TCCCTGTCTGGTGGTCAGAGGGCCAGGATC |
| | | AATTTGGCCAGAGCTGTTTACCGCGAGGCT |
| | | GATATTTACTTGCTTGACGATCCTCTATCG |
| | | GCTGTAGATGCAAACGTCGGCAGGCAACTA |
| | | TTCGACGGCTGCATCAAAGGATACCTAAGC |
| | | GGCAAGACATGCATCCTGGTCACCCATCAG |
| | | ATTCACTACCTTAAAGCTGCAGACTTTATC |
| | | GTAGTCCTAAACGAGGGTTCCGTCGAAAAT |
| | | ATGGGCTCGTATGATGAACTAATGAAAACT |
| | | GGAACGGAATTCTCGATGCTGCTCTCTGAC |
| | | CAAGCTAGTGAAGGCTCTGACACTGACAAA |
| | | AAAGAACGGCCAGCAATGATGCGAGGAATA |
| | | TCAAAGATGTCAGTCAAGAGTGACGATGAG |
| | | GAAGGTGAGGAGAAGGTTCAAGTATTGGAG |
| | | GCCGAAGAGAGACAGTCGGGCAGTCTGAAG |
| | | TGGGATGTGCTTGGGAGGTACATGAAGTCA |
| | | GTCAACTCCTGGTGCATGGTGGTAATGGCA |
| | | TTCCTCGTGTTGGTGATCACGCAGGGTGCT |
| | | GCCACCACTACCGACTACTGGCTTAGTTTC |
| | | TGGACTAACCAGGTGGACATACAAACTCTA |
| | | CCCGAAGGAGAAAGTAAAAATCCTGAATTG |
| | | AACACTCAAGTTGGTCTGTTAACCACGGGA |
| | | CAGTACCTCATTGTGCACGGCAGTGTAGTA |
| | | CTAGCCATTATAATATTGACGCAAGTCAGA |
| | | ATACTTTCCTTCGTAGTGATGACTATGCGA |
| | | GCTTCGGAAAATCTTCATAACACCATTTAC |
| | | GAGAAATTGATAGTGGCTGTAATGAGATTT |
| | | TTCGATACCAATCCATCGGGTCGTGTCTTG |
| | | AACAGATTCTCAAAAGATATGGGTGCAATG |
| | | GATGAGCTGTTACCGCGAAGTATGTTGGAA |
| | | ACGGTTCAGATGTACCTGTCTCTAGCCAGT |
| | | GTCCTTGTGCTGAACGCGATAGCCTTACCA |
| | | TGGACGTTGATACCCACCACAGTGTTGATG |
| | | TTTATATTTGTATTCCTATTGAAGTGGTAC |
| | | ATTAACGCTGCTCAAGCTGTGAAACGATTG |
| | | GAAGGAACTACAAAGAGTCCAGTGTTTGGA |
| | | ATGATCAACTCTACTATATCTGGACTCTCC |
| | | ACCATAAGAAGCTCCAACTCTCAGGACCGA |
| | | CTTCTAAACTCATTTGATGATGCACAGAAT |
| | | CTCCATACTAGCGCTTTCTACACGTTTTTG |
| | | GGTGGTTCAACCGCATTTGGTCTGTACCTG |
| | | GACACCCTCTGTTTAATCTACCTCGGAATT |
| | | ATCATGTCCATCTTCATTCTTGGTGATTTC |
| | | GGTGAGCTGATACCGGTCGGTAGTGTGGGT |
| | | CTGGCCGTCAGTCAGTCCATGGTGCTCACC |
| | | ATGATGTTGCAAATGGCCGCCAAGTTCACT |
| | | GCCGACTTCTTGGGACAGATGACAGCCGTC |
| | | GAGAGGGTCCTGGAGTACACCAAGCTACCT |
| | | ACCGAAGAAAACATGGAGACTGGACCGACG |
| | | ACGCCACCAAAAGACTGGCCAAGTGCTGGA |
| | | GAGGTGACGTTCTCAAACGTGTACCTCAAG |
| | | TACTCACCTGACGACCCACCTGTACTGAAG |
| | | GACCTGAACTTTGCAATCAAGAGTGGATGG |
| | | AAGGTCGGAGTAGTTGGTAGAACTGGTGCT |
| | | GGCAAGTCATCGTTGATATCAGCTCTGTTC |
| | | CGGCTCAGCGACATTACAGGCAGCATCAAA |
| | | ATTGATGGCCTCGATACTCAAGGAATTGCC |
| | | AAGAAGCTTTTGAGATCAAAAATATCAATA |
| | | ATTCCGCAAGAGCCAGTGTTGTTCTCTGCC |
| | | TCGCTGCGTTACAATCTGGACCCATTCGAC |
| | | AACTACAACGATGAAGATATTTGGAGAGCA |
| | | TTGGAACAGGTGGAACTAAAGGAGAGTATA |
| | | CCAGCCCTCGATTACAAAGTGTCGGAAGGC |
| | | GGCACCAACTTCTCGATGGGCCAACGTCAG |
| | | CTGGTATGCCTAGCGCGCGCCATCCTCCGC |
| | | TCAAATAAAATTCTCATCATGGACGAAGCT |
| | | ACCGCTAACGTCGATCCTCAGACGGATGCT |
| | | TTGATCCAGAAAACAATTCGTAAACAATTT |
| | | GCAACGTGCACGGTGCTCACGATCGCGCAT |
| | | CGACTGAACACCATTATGGATTCAGATCGA |
| | | GTACTAGTCATGGACCAGGGAGTGGCCGCG |
| | | GAGTTCGACCACCCCTACATCTTGCTATCT |
| | | AACCCCAATAGCAAGTTCTCTTCAATGGTG |
| | | AAGGAAACAGGCGACAACATGTCCCGGATC |
| | | TTGTTCGAAGTAGCCAAAACAAAATATGAA |
| | | AGTGATTCCAAAACTGCTGACTACAAAGAC |
| | | GATGACGACAAGTAG |
| 13 | Sf-ABCC3 | MDTKKGSKNDSKSKPPPPKPKSVPNVFKRT |
| | | LFCWMLPIFYFGNRRDLEESDLPPPKNMYQ |
| | | SKMLGDKLERSWLKEEHEAKLAGRKPKFTK |
| | | VLFKTFIWSYIPGGLMQATSIGLRTASPLL |
| | | FSQLLRYWAADSPVDRETAMYYAISMILAN |
| | | WASSFMNHHGVLFCQQFGMKLRCAVGSLMF |
| | | RKIMRMSNGSLGDTAAGKVVNILSNDLQRF |
| | | DLSMVFLHYVWIIPLQIAAVIYLGYLQAGT |
| | | AAFIGFAALIIIAINNFPGGLGQYLGKIRL |
| | | RTAEKTDNRIKIMSEVINGIQVIKMYAWEI |
| | | PFQKVVGQKRAEELKEVKIAXLRTVFLGFM |
| | | MFTERAALFFTVLTYVLLGNXMSANVFYPL |
| | | QQFMSAAQVNITLILPMVLSFTAELFVSLG |
| | | RVEKFLLLEDRPDLKGHPEETSSNLFRNMS |
| | | ADGFPETNGSIRPLSYHRKSEASPIEPGSE |
| | | IQLKVPKFARSVSYQEDSALVLHDVSASWT |
| | | GDPNMMALKNISMRLRKGKLCAIIGAVGSG |
| | | KSSILQLLLKELPSATGTISIYGKMSYACQ |
| | | EAWLFPSTVRENILFGLPFEPGKYKKVCRV |
| | | CALEKDFKQFPYGDQTLVGERGVSLSGGQR |
| | | ARINLARAVYREADIYLLDDPLSAVDANVG |
| | | RQLFEGCINGYLRGRTRILVTHQIHFLKAA |
| | | DYIVVLNEGRIENMGTFDELVSSGKEFSMM |
| | | LSQLQEGKEKDTESVGSRSIEEKEKPTLKT |
| | | MISVNEGEEVQEFEAQKMKEEERQSGNLRW |
| | | EVISAYFRSGGHVCFILFALLVVVLATTCA |
| | | ASVDYWVSYWTNQMAAYEESLGGESIEPGL |
| | | DVQAGRFTVGQYLIIHGCLVAALVLMVNLR |
| | | VFPFAYLCVSASAKLHNQMFSTMLRGVMRF |
| | | FDTSSSGRILNRFTKDMGSLDEILPRTLLD |
| | | VLQIYGTLTAILVLNAIALYWTLVPSAVLL |
| | | VLFGFMVRIYLKAAQGIKRLEGTTKSPMFG |
| | | TVTSSLSGISTIRASNAQERLIEQFDINQV |
| | | SHTTSWNSYLNGGTTFGFYLDTMCLVYMTT |
| | | IIFVFLFIDFGDAIPVGSVGLAVTQSNTLT |
| | | LMLQHGARMLVEFLAQLTSVERILEYTRID |
| | | TEPDLFQGKVEMPPNWPYQGRIEFQNVSLR |
| | | YAPNEQPVLKNLNIVIESGNKIGIVGRTGA |
| | | GKSSLISALFRFAYLDGLISIDGLDTSLIS |
| | | RQGLRSKISIIPQEPILFSATIRYNLDPFD |
| | | IYSDDDLWRALEQVDLKSAVPSLDFKVTEG |
| | | GSNFSVGQRQLMCLARAVLRSNQILIMDEA |
| | | TANVDPQTDNFIQETIRRQFVSCTVLTIAH |
| | | RLNTIMDSDKVLVMSSGQVAEYDHPYVLLS |
| | | DPNSHFSAMVRETGEKNSANLFQVAKDAYF |
| | | QSNLKENARDYKDDDDK |
| 14 | Sf-ABCC3 | ATGGATACAAAGAAAGGAAGTAAAAATGAC |
| | | TCGAAATCCAAACCGCCGCCGCCTAAACCA |
| | | AAGAGTGTGCCAAACGTGTTTAAGAGGACA |
| | | TTGTTCTGCTGGATGCTGCCTATATTTTAC |
| | | TTTGGCAACCGCAGAGACCTGGAGGAAAGT |
| | | GATCTTCCACCTCCTAAGAATATGTATCAA |
| | | TCCAAGATGCTCGGAGATAAATTGGAAAGG |
| | | AGTTGGCTGAAAGAAGAACATGAAGCGAAA |
| | | CTAGCCGGACGAAAGCCGAAATTTACGAAG |
| | | GTTTTATTCAAAACATTCATTTGGTCTTAT |
| | | ATACCAGGCGGTTTGATGCAGGCTACCTCA |
| | | ATTGGCCTACGTACGGCATCTCCGCTGCTG |
| | | TTCAGCCAACTGCTCCGATATTGGGCAGCT |
| | | GACTCCCCGGTGGACCGGGAGACGGCCATG |
| | | TACTACGCGATCAGTATGATCCTCGCCAAC |
| | | TGGGCGTCATCCTTCATGAACCACCACGGA |
| | | GTACTGTTCTGTCAGCAGTTTGGTATGAAG |
| | | CTGAGGTGTGCCGTCGGATCTTTAATGTTC |
| | | AGAAAGATAATGCGTATGAGCAACGGCTCT |
| | | CTAGGTGACACTGCAGCGGGTAAGGTAGTG |
| | | AACATATTATCGAACGACCTGCAGAGGTTC |
| | | GACTTGTCAATGGTGTTCCTCCACTACGTA |
| | | TGGATTATTCCGCTACAAATTGCCGCAGTC |
| | | ATATATCTGGGCTACCTACAAGCTGGAACC |
| | | GCGGCTTTCATTGGATTCGCTGCCTTAATC |
| | | ATCATAGCCATAAATAATTTTCCAGGTGGT |
| | | TTGGGCCAGTACCTAGGTAAAATTCGACTT |
| | | CGCACCGCTGAAAAAACAGACAACAGAATT |
| | | AAAATCATGAGCGAAGTTATAAACGGAATA |
| | | CAGGTGATTAAAATGTACGCATGGGAGATT |
| | | CCTTTCCAAAAAGTAGTCGGCCAGAAACGT |
| | | GCGGAAGAGTTGAAGGAAGTGAAGATTGCT |
| | | NNTCTGAGGACCGTGTTCTTAGGATTTATG |
| | | ATGTTCACCGAAAGAGCTGCGCTTTTCTTC |
| | | ACCGTACTCACTTATGTCTTGTTGGGGAAC |
| | | NGTATGTCTGCTAATGTGTTCTACCCGCTG |
| | | CAGCAATTTATGAGCGCCGCTCAAGTAAAC |
| | | ATTACATTAATCTTGCCTATGGTGCTATCG |
| | | TTCACTGCTGAGCTCTTCGTGTCCCTTGGA |
| | | AGAGTCGAGAAGTTCCTGTTACTAGAGGAC |
| | | AGGCCAGACTTGAAGGGGCATCCGGAAGAA |
| | | ACATCGTCCAATTTATTCCGCAACATGTCG |
| | | GCCGATGGCTTCCCGGAGACCAACGGCTCT |
| | | ATCCGACCGCTCTCCTACCATCGGAAATCT |
| | | GAAGCATCACCAATAGAGCCGGGTAGTGAG |
| | | ATACAGCTGAAGGTTCCTAAGTTTGCACGG |
| | | AGTGTGTCGTATCAGGAGGACTCAGCGCTA |
| | | GTGTTACACGATGTGTCAGCCAGTTGGACC |
| | | GGGGACCCCAACATGATGGCGCTGAAGAAC |
| | | ATCTCCATGCGACTGCGGAAGGGAAAGCTG |
| | | TGCGCTATCATTGGTGCAGTCGGTTCTGGG |
| | | AAGTCCTCGATTTTGCAACTACTACTAAAA |
| | | GAATTGCCTTCGGCGACTGGCACTATTTCA |
| | | ATATATGGCAAGATGTCGTACGCTTGCCAA |
| | | GAGGCGTGGCTGTTCCCCAGCACTGTCCGG |
| | | GAGAATATACTTTTCGGTTTGCCGTTTGAA |
| | | CCCGGAAAATATAAGAAGGTGTGCCGAGTG |
| | | TGTGCACTCGAGAAGGACTTCAAGCAGTTC |
| | | CCGTACGGGGACCAGACGTTGGTGGGGGAG |
| | | AGGGGGGTGTCCCTCTCCGGAGGCCAACGT |
| | | GCCCGAATCAATTTGGCGCGCGCTGTTTAC |
| | | AGAGAGGCCGATATTTACCTTCTCGATGAT |
| | | CCGCTGTCGGCTGTGGACGCTAACGTTGGA |
| | | CGTCAGCTATTTGAGGGATGTATCAACGGT |
| | | TACCTGCGTGGGCGCACTAGGATCCTGGTC |
| | | ACACATCAGATTCACTTCTTGAAAGCTGCT |
| | | GATTACATCGTIGTACTAAACGAGGGCCGT |
| | | ATTGAAAATATGGGAACATTTGACGAATTG |
| | | GTTTCGTCCGGCAAAGAATTCTCAATGATG |
| | | CTCTCTCAGCTACAGGAAGGCAAAGAAAAG |
| | | GACACGGAGAGTGTTGGGTCACGGAGTATC |
| | | GAAGAAAAAGAAAAACCAACTCTAAAGACC |
| | | ATGATCAGTGTGAATGAAGGTGAGGAGGTA |
| | | CAAGAGTTTGAAGCACAGAAGATGAAGGAA |
| | | GAAGAGAGACAATCTGGCAACCTCCGCTGG |
| | | GAAGTGATCTCTGCGTACTTCCGATCGGGA |
| | | GGGCACGTCTGTTTCATCTTATTTGCACTC |
| | | TTGGTCGTTGTACTGGCCACTACCTGCGCC |
| | | GCTTCTGTTGACTACTGGGTCAGTTATTGG |
| | | ACCAATCAAATGGCTGCATATGAGGAGTCC |
| | | TTAGGCGGAGAAAGTATCGAACCTGGCTTG |
| | | GACGTGCAGGCGGGTCGGTTCACAGTGGGA |
| | | CAGTATCTGATCATCCACGGCTGCCTGGTG |
| | | GCTGCCTTGGTGTTGATGGTCAACCTGCGA |
| | | GTCTTCCCCTTCGCCTACCTCTGTGTATCC |
| | | GCCTCAGCCAAGCTACATAACCAGATGTTC |
| | | TCGACTATGCTGAGAGGAGTCATGAGGTTC |
| | | TTCGACACCAGTTCCTCAGGTCGTATCTTG |
| | | AATCGTTTTACCAAAGACATGGGTTCTCTC |
| | | GATGAGATCCTGCCTCGTACCTTACTGGAT |
| | | GTACTACAGATCTACGGAACATTGACTGCT |
| | | ATCCTGGTACTCAACGCCATTGCTCTATAC |
| | | TGGACCCTGGTACCTTCAGCAGTTCTCCTA |
| | | GTGTTATTCGGCTTTATGGTACGAATATAC |
| | | TTGAAGGCAGCTCAAGGCATTAAACGATTG |
| | | GAAGGCACAACAAAGAGTCCAATGTTTGGA |
| | | ACTGTGACGTCATCATTGAGTGGTATTTCC |
| | | ACTATCAGGGCATCCAATGCTCAAGAGAGA |
| | | TTGATAGAACAGTTTGACATCAACCAGGTA |
| | | AGTCATACTACTTCGTGGAACAGTTACTTG |
| | | AATGGAGGAACGACGTTTGGTTTCTATTTA |
| | | GATACAATGTGCCTGGTATACATGACTACT |
| | | ATCATATTTGTGTTCCTGTTCATCGATTTC |
| | | GGGGACGCGATCCCAGTGGGCAGCGTAGGG |
| | | CTAGCGGTGACGCAGAGCAACACACTGACG |
| | | CTGATGCTGCAGCACGGCGCGCGGATGTTG |
| | | GTCGAGTTCCTCGCGCAGCTCACCAGCGTC |
| | | GAGCGCATCCTCGAGTACACGCGCATCGAC |
| | | ACCGAGCCAGACTTGTTCCAAGGAAAGGTT |
| | | GAAATGCCGCCAAATTGGCCCTACCAAGGA |
| | | AGGATCGAGTTTCAAAACGTTTCTCTACGG |
| | | TACGCTCCAAATGAACAACCCGTCCTTAAA |
| | | AATTTGAACATTGTTATCGAAAGCGGAAAC |
| | | AAGATCGGGATCGTGGGGCGGACGGGCGCC |
| | | GGCAAATCTTCTTTGATATCAGCATTATTC |
| | | CGATTCGCCTACCTCGACGGATTAATATCC |
| | | ATAGATGGACTCGATACTTCGCTGATTTCG |
| | | AGACAGGGACTGCGATCGAAAATTTCAATT |
| | | ATTCCGCAAGAACCGATCCTGTTCTCTGCG |
| | | ACAATCAGATACAATTTGGATCCGTTTGAC |
| | | ATTTACAGCGACGACGACCTCTGGAGAGCC |
| | | CTCGAACAGGTCGACTTGAAGTCTGCTGTA |
| | | CCGTCCTTGGACTTCAAAGTAACAGAAGGA |
| | | GGTTCTAATTTCTCAGTGGGCCAGCGGCAG |
| | | CTGATGTGTTTGGCGCGCGCTGTGTTGAGA |
| | | TCCAATCAGATCTTAATTATGGACGAAGCT |
| | | ACGGCGAACGTAGATCCTCAGACGGATAAC |
| | | TTCATTCAGGAGACGATTCGCCGGCAGTTC |
| | | GTGTCGTGCACCGTGCTGACGATAGCTCAC |
| | | AGGTTGAACACGATCATGGACTCCGACAAG |
| | | GTGCTCGTGATGAGCAGCGGCCAGGTGGCG |
| | | GAGTACGACCACCCGTACGTGCTGCTCTCC |
| | | GACCCTAACAGTCACTTCTCCGCCATGGTG |
| | | CGGGAGACCGGCGAGAAGAACAGTGCTAAC |
| | | TTATTCCAAGTAGCCAAAGACGCTTACTTC |
| | | CAAAGTAATCTGAAAGAAAACGCGAGAGAC |
| | | TACAAAGACGATGACGACAAGTAG |
| 15 | CotC | MGYYKKYKEEYYTVKKTYYKKYYEYDKKDY |
| | | DCDYDKKYDDYDKKYYDHDKKDYDYVVEYK |
| | | KHKKHY |
| 16 | cotC | GGATAAATCGTTTGGGCCGATGAAAAATCG |
| | | GCTCTTTATTTTGATTTGTTTTTGTGTCAT |
| | | CTGTCTTTTTCTATCATTTGGACAGCCCTT |
| | | TTTTCCTTCTATGATTTTAACTGTCCAAGC |
| | | CGCAAAATCTACTCGCCGTATAATAAAGCG |
| | | TAGTAAAAATAAAGGAGGAGTATATATGGG |
| | | TTATTACAAAAAATACAAAGAAGAGTATTA |
| | | TACGGTCAAAAAAACGTATTATAAGAAGTA |
| | | TTACGAATATGATAAAAAAGATTATGACTG |
| | | TGATTACGACAAAAAATATGATGACTATGA |
| | | TAAAAAATATTATGATCACGATAAAAAAGA |
| | | CTATGATTATGTTGTAGAGTATAAAAAGCA |
| | | TAAAAAACACTACTAA |
| 17 | CotG | MGHYSHSDIEEAVKSAKKEGLKDYLYQEPH |
| | | GKKRSHKKSHRTHKKSRSHKKSYCSHKKSR |
| | | SHKKSFCSHKKSRSHKKSYCSHKKSRSHKK |
| | | SYRSHKKSRSYKKSYRSYKKSRSYKKSCRS |
| | | YKKSRSYKKSYCSHKKKSRSYKKSCRTHKK |
| | | SYRSHKKYYKKPHHHCDDYKRHDDYDSKKE |
| | | YWKDGNCWVVKKKYK |
| 18 | cotG | AAATTGGATTGATTCTTCATCCATAATCCT |
| | | CCTTACAAATTTTAGGCTTTTATTTTTATA |
| | | AGATCTCAGCGGAACACTTATACACTTTTT |
| | | AAAACCGCGCGTACTATGAGGGTAGTAAGG |
| | | ATCTTCATCCTTAACATATTTTTAAAAGGA |
| | | GGATTTCAAAATGGGCCACTATTCCCATTC |
| | | TGACATCGAAGAAGCGGTGAAATCCGCAAA |
| | | AAAAGAAGGTTTAAAGGATTATTTATACCA |
| | | AGAGCCTCATGGAAAAAAACGCAGTCATAA |
| | | AAAGTCGCACCGCACTCACAAAAAATCTCG |
| | | CAGCCATAAAAAATCATACTGCTCTCACAA |
| | | AAAATCTCGCAGTCACAAAAAATCATTCTG |
| | | TTCTCACAAAAAATCTCGCAGCCACAAAAA |
| | | ATCATACTGCTCTCACAAGAAATCTCGCAG |
| | | CCACAAAAAATCGTACCGTTCTCACAAAAA |
| | | ATCTCGCAGCTATAAAAAATCTTACCGTTC |
| | | TTACAAAAAATCTCGTAGCTATAAAAAATC |
| | | TTGCCGTTCTTACAAAAAATCTCGCAGCTA |
| | | CAAAAAGTCTTACTGTTCTCACAAGAAAAA |
| | | ATCTCGCAGCTATAAGAAGTCATGCCGCAC |
| | | ACACAAAAAATCTTATCGTTCCCATAAGAA |
| | | ATACTACAAAAAACCGCACCACCACTGCGA |
| | | CGACTACAAAAGACACGATGATTATGACAG |
| | | CAAAAAAGAATACTGGAAAGACGGCAATTG |
| | | CTGGGTAGTCAAAAAGAAATACAAATAA |
| 19 | cry1Ac-Term | TCTAGAGTCCTCATGCAAACTCAGGTTTAA |
| | | ATATCGTTTTCAAATCAATTGTCCAAGAGC |
| | | AGCATTACAAATAGATAAGTAATTTGTTGT |
| | | AATGAAAAACGGACATCACCTCCATTGAAA |
| | | CGGAGTGATGTCCGTTTTACTATGTTATTT |
| | | TCTAGTAATACATATGTATAGAGCAACTTA |
| | | ATCAAGCAGAGATATTTTCACCTATCGATG |
| | | AAAATATCTCTGCTTTTTCTTTTTTTATTT |
| | | GGTATATGCTTTACTTGTAATCGAAAATAA |
| | | AGCACTAATAGGGTGTTTTTGCCCATCCCC |
| | | TCGAAAAGGGGGAATAAGAAAAAATAGGAT |
| | | GTTTTTGTAGATGGAGCGCCAGAGGACCTG |
| | | CAGGCATGCAAGCTCTAGA |
| 20 | Cry 1 Aa Leader | MDNNPNINECIPYNCLSNPEVEVLGGER |
| 21 | Cry 1Fa Leader | MENNIQNQCVPYNCLNNPEVEILNEER |
| 22 | pSB634 | GCGGCCGCCACCGCGGTGGAGCTCCAATTC |
| | | GCCCTATAGTGAGTCGTATTACGCGCGCTC |
| | | ACTGGCCGTCGTTTTACAACGTCGTGACTG |
| | | GGAAAACCCTGGCGTTACCCAACTTAATCG |
| | | CCTTGCAGCACATCCCCCTTTCGCCAGCTG |
| | | GCGTAATAGCGAAGAGGCCCGCACCGATCG |
| | | CCCTTCCCAACAGTTGCGCAGCCTGAATGG |
| | | CGAATGGGACGCGCCCTGTAGCGGCGCATT |
| | | AAGCGCGGCGGGTGTGGTGGTTACGCGCAG |
| | | CGTGACCGCTACACTTGCCAGCGCCCTAGC |
| | | GCCCGCTCCTTTCGCTTTCTTCCCTTCCTT |
| | | TCTCGCCACGTTCGCCGGCTTTCCCCGTCA |
| | | AGCTCTAAATCGGGGGCTCCCTTTAGGGTT |
| | | CCGATTTAGTGCTTTACGGCACCTCGACCC |
| | | CAAAAAACTTGATTAGGGTGATGGTTCACG |
| | | TAGTGGGCCATCGCCCTGATAGACGGTTTT |
| | | TCGCCCTTTGACGTTGGAGTCCACGTTCTT |
| | | TAATAGTGGACTCTTGTTCCAAACTGGAAC |
| | | AACACTCAACCCTATCTCGGTCTATTCTTT |
| | | TGATTTATAAGGGATTTTGCCGATTTCGGC |
| | | CTATTGGTTAAAAAATGAGCTGATTTAACA |
| | | AAAATTTAACGCGAATTTTAACAAAATATT |
| | | AACGCTTACAATTTAGGTGGCACTTTTCGG |
| | | GGAAATGTGCGCGGAACCCCTATTTGTTTA |
| | | TTTTTCTAAATACATTCAAATATGTATCCG |
| | | CTCATGAGACAATAACCCTGATAAATGCTT |
| | | CAATAATATTGAAAAAGGAAGAGTATGAGT |
| | | ATTCAACATTTCCGTGTCGCCCTTATTCCC |
| | | TTTTTTGCGGCATTTTGCCTTCCTGTTTTT |
| | | GCTCACCCAGAAACGCTGGTGAAAGTAAAA |
| | | GATGCTGAAGATCAGTTGGGTGCACGAGTG |
| | | GGTTACATCGAACTGGATCTCAACAGCGGT |
| | | AAGATCCTTGAGAGTTTTCGCCCCGAAGAA |
| | | CGTTTTCCAATGATGAGCACTTTTAAAGTT |
| | | CTGCTATGTGGCGCGGTATTATCCCGTATT |
| | | GACGCCGGGCAAGAGCAACTCGGTCGCCGC |
| | | ATACACTATTCTCAGAATGACTTGGTTGAG |
| | | TACTCACCAGTCACAGAAAAGCATCTTACG |
| | | GATGGCATGACAGTAAGAGAATTATGCAGT |
| | | GCTGCCATAACCATGAGTGATAACACTGCG |
| | | GCCAACTTACTTCTGACAACGATCGGAGGA |
| | | CCGAAGGAGCTAACCGCTTTTTTGCACAAC |
| | | ATGGGGGATCATGTAACTCGCCTTGATCGT |
| | | TGGGAACCGGAGCTGAATGAAGCCAAACCA |
| | | AACGACGAGCGTGACACCACGATGCCTGTA |
| | | GCAATGGCAACAACGTTGCGCAAACTATTA |
| | | ACTGGCGAACTACTTACTCTAGCTTCCCGG |
| | | CAACAATTAATAGACTGGATGGAGGCGGAT |
| | | AAAGTTGCAGGACCACTTCTGCGCTCGGCC |
| | | CTTCCGGCTGGCTGGTTTATTGCTGATAAA |
| | | TCTGGAGCCGGTGAGCGTGGGTCTCGCGGT |
| | | ATCATTGCAGCACTGGGGCCAGATGGTAAG |
| | | CCCTCCCGTATCGTAGTTATCTACACGACG |
| | | GGGAGTCAGGCAACTATGGATGAACGAAAT |
| | | AGACAGATCGCTGAGATAGGTGCCTCACTG |
| | | ATTAAGCATTGGTAACTGTCAGACCAAGTT |
| | | TACTCATATATACTTTAGATTGATTTAAAA |
| | | CTTCATTTTTAATTTAAAAGGATCTAGGTG |
| | | AAGATCCTTTTTGATAATCTCATGACCAAA |
| | | ATCCCTTAACGTGAGTTTTCGTTCCACTGA |
| | | GCGTCAGACCCCGTAGAAAAGATCAAAGGA |
| | | TCTTCTTGAGATCCTTTTTTTCTGCGCGTA |
| | | ATCTGCTGCTTGCAAACAAAAAAACCACCG |
| | | CTACCAGCGGTGGTTTGTTTGCCGGATCAA |
| | | GAGCTACCAACTCTTTTTCCGAAGGTAACT |
| | | GGCTTCAGCAGAGCGCAGATACCAAATACT |
| | | GTTCTTCTAGTGTAGCCGTAGTTAGGCCAC |
| | | CACTTCAAGAACTCTGTAGCACCGCCTACA |
| | | TACCTCGCTCTGCTAATCCTGTTACCAGTG |
| | | GCTGCTGCCAGTGGCGATAAGTCGTGTCTT |
| | | ACCGGGTTGGACTCAAGACGATAGTTACCG |
| | | GATAAGGCGCAGCGGTCGGGCTGAACGGGG |
| | | GGTTCGTGCACACAGCCCAGCTTGGAGCGA |
| | | ACGACCTACACCGAACTGAGATACCTACAG |
| | | CGTGAGCTATGAGAAAGCGCCACGCTTCCC |
| | | GAAGGGAGAAAGGCGGACAGGTATCCGGTA |
| | | AGCGGCAGGGTCGGAACAGGAGAGCGCACG |
| | | AGGGAGCTTCCAGGGGGAAACGCCTGGTAT |
| | | CTTTATAGTCCTGTCGGGTTTCGCCACCTC |
| | | TGACTTGAGCGTCGATTTTTGTGATGCTCG |
| | | TCAGGGGGGCGGAGCCTATGGAAAAACGCC |
| | | AGCAACGCGGCCTTTTTACGGTTCCTGGCC |
| | | TTTTGCTGGCCTTTTGCTCACATGTTCTTT |
| | | CCTGCGTTATCCCCTGATTCTGTGGATAAC |
| | | CGTATTACCGCCTTTGAGTGAGCTGATACC |
| | | GCTCGCCGCAGCCGAACGACCGAGCGCAGC |
| | | GAGTCAGTGAGCGAGGAAGCGGAAGAGCGC |
| | | CCAATACGCAAACCGCCTCTCCCCGCGCGT |
| | | TGGCCGATTCATTAATGCAGCTGGCACGAC |
| | | AGGTTTCCCGACTGGAAAGCGGGCAGTGAG |
| | | CGCAACGCAATTAATGTGAGTTAGCTCACT |
| | | CATTAGCACCCCAGGCTTTACACTTTATGC |
| | | TCCCGGCTCGTATGTTGTGTGGAATTGTGA |
| | | GCGGATAACAATTTCACACAGGAAACAGCT |
| | | ATGACCATGATTACGCCAAGCGCGCAATTA |
| | | ACCCTCACTAAAGGGAACAAAAGCTGGGTA |
| | | CGGCGCGCCTTAAGGAACGTACAGACGGCT |
| | | TAAAAGCCTTTAAAAACGTTTTTAAGGGGT |
| | | TTGTAGACAAGGTAAAGGATAAAACAGCAC |
| | | AATTCCAAGAAAAACACGATTTAGAACCTA |
| | | AAAAGAACGAATTTGAACTAACTCATAACC |
| | | GAGAGGTAAAAAAAGAACGAAGTCGAGATC |
| | | AGGGAATGAGTTTATAAAATAAAAAAAGCA |
| | | CCTGAAAAGGTGTCTTTTTTTGATGGTTTT |
| | | GAACTTGTTCTTTCTTATCTTGATACATAT |
| | | AGAAATAACGTCATTTTTATTTTAGTTGCT |
| | | GAAAGGTGCGTTGAAGTGTTGGTATGTATG |
| | | TGTTTTAAAGTATTGAAAACCCTTAAAATT |
| | | GGTTGCACAGAAAAACCCCATCTGTTAAAG |
| | | TTATAAGTGACCAAACAAATAACTAAATAG |
| | | ATGGGGGTTTCTTTTAATATTATGTGTCCT |
| | | AATAGTAGCATTTATTCAGATGAAAAATCA |
| | | AGGGTTTTAGTGGACAAGACAAAAAGTGGA |
| | | AAAGTGAGACCATGGAGAGAAAAGAAAATC |
| | | GCTAATGTTGGTTACTTTGAACTTCTGCAT |
| | | ATTCTTGAATTTAAAAAGGCTGAAAGAGTA |
| | | AAAGATTGTGCTGAAATATTAGAGTATAAA |
| | | CAAAATCGTGAAACAGGCGAAAGAAAGTTG |
| | | TATCGAGTGTGGTTTTGTAAATCCAGGCTT |
| | | TGTCCAATGTGCAACTGGAGGAGAGCAATG |
| | | AAACATGGCATTCAGTCACAAAAGGTTGTT |
| | | GCTGAAGTTATTAAACAAAAGCCAACAGTT |
| | | CGTTGGTTGTTTCTCACATTAACAGTTAAA |
| | | AATGTTTATGATGGCGAAGAATTAAATAAG |
| | | AGTTTGTCAGATATGGCTCAAGGATTTCGC |
| | | CGAATGATGCAATATAAAAAAATTAATAAA |
| | | AATCTTGTTGGTTTTATGCGTGCAACGGAA |
| | | GTGACAATAAATAATAAAGATAATTCTTAT |
| | | AATCAGCACATGCATGTATTGGTATGTGTG |
| | | GAACCAACTTATTTTAAGAATACAGAAAAC |
| | | TACGTGAATCAAAAACAATGGATTCAATTT |
| | | TGGAAAAAGGCAATGAAATTAGACTATGAT |
| | | CCAAATGTAAAAGTTCAAATGATTCGACCG |
| | | AAAAATAAATATAAATCGGATATACAATCG |
| | | GCAATTGACGAAACTGCAAAATATCCTGTA |
| | | AAGGATACGGATTTTATGACCGATGATGAA |
| | | GAAAAGAATTTGAAACGTTTGTCTGATTTG |
| | | GAGGAAGGTTTACACCGTAAAAGGTTAATC |
| | | TCCTATGGTGGTTTGTTAAAAGAAATACAT |
| | | AAAAAATTAAACCTTGATGACACAGAAGAA |
| | | GGCGATTTGATTCATACAGATGATGACGAA |
| | | AAAGCCGATGAAGATGGATTTTCTATTATT |
| | | GCAATGTGGAATTGGGAACGGAAAAATTAT |
| | | TTTATTAAAGAGTAGTTCAACAAACGGGCC |
| | | ATATTGTTGTATAAGTGATGAAATACTGAA |
| | | TTTAAAACTTAGTTTATATGTGGTAAAATG |
| | | TTTTAATCAAGTTTAGGAGGAATTAATTAT |
| | | GAAGTGTAATGAATGTAACAGGGTTCAATT |
| | | AAAAGAGGGAAGCGTATCATTAACCCTATA |
| | | AACTACGTCTGCCCTCATTATTGGAGGGTG |
| | | AAATGTGAATACATCCTATTCACAATCGAA |
| | | TTTACGACACAACCAAATTTTAATTTGGCT |
| | | TTGCATTTTATCTTTTTTTAGCGTATTAAA |
| | | TGAAATGGTTTTGAACGTCTCATTACCTGA |
| | | TATTGCAAATGATTTTAATAAACCACCTGC |
| | | GAGTACAAACTGGGTGAACACAGCCTTTAT |
| | | GTTAACCTTTTCCATTGGAACAGCTGTATA |
| | | TGGAAAGCTATCTGATCAATTAGGCATCAA |
| | | AAGGTTACTCCTATTTGGAATTATAATAAA |
| | | TTGTTTCGGGTCGGTAATTGGGTTTGTTGG |
| | | CCATTCTTTCTTTTCCTTACTTATTATGGC |
| | | TCGTTTTATTCAAGGGGCTGGTGCAGCTGC |
| | | ATTTCCAGCACTCGTAATGGTTGTAGTTGC |
| | | GCGCTATATTCCAAAGGAAAATAGGGGTAA |
| | | AGCATTTGGTCTTATTGGATCGATAGTAGC |
| | | CATGGGAGAAGGAGTCGGTCCAGCGATTGG |
| | | TGGAATGATAGCCCATTATATTCATTGGTC |
| | | CTATCTTCTACTCATTCCTATGATAACAAT |
| | | TATCACTGTTCCGTTTCTTATGAAATTATT |
| | | AAAGAAAGAAGTAAGGATAAAAGGTCATTT |
| | | TGATATCAAAGGAATTATACTAATGTCTGT |
| | | AGGCATTGTATTTTTTATGTTGTTTACAAC |
| | | ATCATATAGCATTTCTTTTCTTATCGTTAG |
| | | CGTGCTGTCATTCCTGATATTTGTAAAACA |
| | | TATCAGGAAAGTAACAGATCCTTTTGTTGA |
| | | TCCCGGATTAGGGAAAAATATACCTTTTAT |
| | | GATTGGAGTTCTTTGTGGGGGAATTATATT |
| | | TGGAACAGTAGCAGGGTTTGTCTCTATGGT |
| | | TCCTTATATGATGAAAGATGTTCACCAGCT |
| | | AAGTACTGCCGAAATCGGAAGTGTAATTAT |
| | | TTTCCCTGGAACAATGAGTGTCATTATTTT |
| | | CGGCTACATTGGTGGGATACTTGTTGATAG |
| | | AAGAGGTCCTTTATACGTGTTAAACATCGG |
| | | AGTTACATTTCTTTCTGTTAGCTTTTTAAC |
| | | TGCTTCCTTTCTTTTAGGAACAACATCATG |
| | | GTTCATGACAATTATAATCGTATTTGTTTT |
| | | AGGTGGCCTTTCGTTCACCAAAACAGTTAT |
| | | ATCAACAATTGTTTCAAGTAGCTTGAAACA |
| | | GCAGGAAGCTGGTGCTGGAATGAGTTTGCT |
| | | TAACTTTACCAGCTTTTTATCAGAGGGAAC |
| | | AGGTATTGCAATTGTAGGTGGTTTATTATC |
| | | CATACCCTTACTTGATCAAAGGTTGTTACC |
| | | TATGGAAGTTGATCAGTCAACTTATCTGTA |
| | | TAGTAATTTGTTATTACTTTTTTCAGGAAT |
| | | CATTGTCATTAGTTGGCTGGTTACCTTGAA |
| | | TGTATATAAACATTCTCAAAGGGATTTCTA |
| | | AATCGTTAAGGGATCAACTTTGGGAGAGAG |
| | | TTCAAAATTGATCCTTTTTTTATAACAGGA |
| | | ATTTAACCGTACCGGGCCCCCCCTCGAG |
| 23 | pHY300PLK | AAGCTTCTAGAGATCTGCAGGTCGACGGAT |
| | | CCCCGGGAATTCCTGTTATAAAAAAAGGAT |
| | | CAATTTTGAACTCTCTCCCAAAGTTGATCC |
| | | CTTAACGATTTAGAAATCCCTTTGAGAATG |
| | | TTTATATACATTCAAGGTAACCAGCCAACT |
| | | AATGACAATGATTCCTGAAAAAAGTAATAA |
| | | CAAATTACTATACAGATAAGTTGACTGATC |
| | | AACTTCCATAGGTAACAACCTTTGATCAAG |
| | | TAAGGGTATGGATAATAAACCACCTACAAT |
| | | TGCAATACCTGTTCCCTCTGATAAAAAGCT |
| | | GGTAAAGTTAAGCAAACTCATTCCAGCACC |
| | | AGCTTCCTGCTGTTTCAAGCTACTTGAAAC |
| | | AATTGTTGATATAACTGTTTTGGTGAACGA |
| | | AAGCCCACCTAAAACAAATACGATTATAAT |
| | | TGTCATGAACCATGATGTTGTTTCTAAAAG |
| | | AAAGGAAGCAGTTAAAAAGCTAACAGAAAG |
| | | AAATGTAACTCCGATGTTTAACACGTATAA |
| | | AGGACCTCTTCTATCAACAAGTATCCCACC |
| | | AATGTAGCCGAAAATAATGACACTCATTGT |
| | | TCCAGGGAAAATAATTACACTTCCGATTTC |
| | | GGCAGTACTTAGCTGGTGAACATCTTTCAT |
| | | CATATAAGGAACCATAGAGACAAACCCTGC |
| | | TACTGTTCCAAATATAATTCCCCCACAAAG |
| | | AACTCCAATCATAAAAGGTATATTTTTCCC |
| | | TAATCCGGGATCAACAAAAGGATCTGTTAC |
| | | TTTCCTGATATGTTTTACAAATATCAGGAA |
| | | TGACAGCACGCTAACGATAAGAAAAGAAAT |
| | | GCTATATGATGTTGTAAACAACATAAAAAA |
| | | TACAATGCCTACAGACATTAGTATAATTCC |
| | | TTTGATATCAAAATGACCTTTTATCCTTAC |
| | | TTCTTTCTTTAATAATTTCATAAGAAACGG |
| | | AACAGTGATAATTGTTATCATAGGAATGAG |
| | | TAGAAGATAGGACCAATGAATATAATGGGC |
| | | TATCATTCCACCAATCGCTGGACCGACTCC |
| | | TTCTCCCATGGCTACTATCGATCCAATAAG |
| | | ACCAAATGCTTTACCCCTATTTTCCTTTGG |
| | | AATATAGCGCGCAACTACAACCATTACGAG |
| | | TGCTGGAAATGCAGCTGCACCAGCCCCTTG |
| | | AATAAAACGAGCCATAATAAGTAAGGAAAA |
| | | GAAAGAATGGCCAACAAACCCAATTACCGA |
| | | CCCGAAACAATTTATTATAATTCCAAATAG |
| | | GAGTAACCTTTTGATGCCTAATTGATCAGA |
| | | TAGCTTTCCATATACAGCTGTTCCAATGGA |
| | | AAAGGTTAACATAAAGGCTGTGTTCACCCA |
| | | GTTTGTACTCGCAGGTGGTTTATTAAAATC |
| | | ATTTGCAATATCAGGTAATGAGACGTTCAA |
| | | AACCATTTCATTTAATACGCTAAAAAAAGA |
| | | TAAAATGCAAAGCCAAATTAAAATTTGGTT |
| | | GTGTCGTAAATTCGATTGTGAATAGGATGT |
| | | ATTCACATTTCACCCTCCAATAATGAGGGC |
| | | AGACGTAGTTTATAGGGTTAATGATACGCT |
| | | TCCCTCTTTTAATTGAACCCTGTTACATTC |
| | | ATTATTCATTACACTTCATAATTAATTCCT |
| | | CCTAAACTTGATTAAAACATTTTACCACAT |
| | | ATAAACTAAGTTTTAAATTCAGTATTTCAT |
| | | CACTTATACAACAATATGGCCCGTTTGTTG |
| | | AACTACTCTTTAATAAAATAATTTTTCCGT |
| | | TCCCAATTCCACATTGCAATAATAGAAAAT |
| | | CCATCTTCATCGGCTTTTTCGTCATCATCT |
| | | GTATGAATCAAATCGCCTTCTTCTGTGTCA |
| | | TCAAGGTTTAATTTTTTATGTATTTCTTTT |
| | | AACAAACCACCATAGGAGATTAACCTTTTA |
| | | CGGTGTAAACCTTCCTCCAAATCAGACAAA |
| | | CGTTTCAAATTCTTTTCTTCATCATCGGTC |
| | | ATAAAATCCGTATCCTTTACAGGATATTTT |
| | | GCAGTTTCGTCAATTGCCGATTGTATATCC |
| | | GATTTATATTTATTTTTCGGTCGAATCATT |
| | | TGAACTTTTACATTTGGATCATAGTCTAAT |
| | | TTCATTGCCTTTTTCCAAAATTGAATCCAT |
| | | TGTTTTTGATTCACGTAGTTTTCTGTATTC |
| | | TTAAAATAAGTTGGTTCCACACATACCAAT |
| | | ACATGCATGTGCTGATTATAAGAATTATCT |
| | | TTATTATTTATTGTCACTTCCGTTGCACGC |
| | | ATAAAACCAACAAGATTTTTATTAATTTTT |
| | | TTATATTGCATCATTCGGCGAAATCCTTGA |
| | | GCCATATCTGACAAACTCTTATTTAATTCT |
| | | TCGCCATCATAAACATTTTTAACTGTTAAT |
| | | GTGAGAAACAACCAACGAACTGTTGGCTTT |
| | | TGTTTAATAACTTCAGCAACAACCTTTTGT |
| | | GACTGAATGCCATGTTTCATTGCTCTCCTC |
| | | CAGTTGCACATTGGACAAAGCCTGGATTTA |
| | | CAAAACCACACTCGATACAACTTTCTTTCG |
| | | CCTGTTTCACGATTTTGTTTATACTCTAAT |
| | | ATTTCAGCACAATCTTTTACTCTTTCAGCC |
| | | TTTTTAAATTCAAGAATATGCAGAAGTTCA |
| | | AAGTAATCAACATTAGCGATTTTCTTTTCT |
| | | CTCCATGGTCTCACTTTTCCACTTTTTGTC |
| | | TTGTCCACTAAAACCCTTGATTTTTCATCT |
| | | GAATAAATGCTACTATTAGGACACATAATA |
| | | TTAAAAGAAACCCCCATCTATTTAGTTATT |
| | | TGTTTGGTCACTTATAACTTTAACAGATGG |
| | | GGTTTTTCTGTGCAACCAATTTTAAGGGTT |
| | | TTCCAATACTTTAAAACACATACATACCAA |
| | | CACTTCAACGCACCTTTCAGCAACTAAAAT |
| | | AAAAATGACGTTATTTCTATATGTATCAAG |
| | | ATAAGAAAGAACAAGTTCAAAACCATCAAA |
| | | AAAAGACACCTTTTCAGGTGCTTTTTTTAT |
| | | TTTATAAACTCATTCCCTGATCTCGACTTC |
| | | GTTCTTTTTTTACCTCTCGGTTATGAGTTA |
| | | GTTCAAATTCGTTCTTTTTAGGTTCTAAAT |
| | | CGTGTTTTTCTTGGAATTGTGCTGTTTTAT |
| | | CCTTTACCTTGTCTACAAACCCCTTAAAAA |
| | | CGTTTTTAAAGGCTTTTAAGCGTCTGTACG |
| | | TTCCTTAAGGAATTATTCCTTAGTGCTTTC |
| | | TAGGTTAATGTCATGATAATAATGGTTTCT |
| | | TAGACGTCAGGTGGCACTTTTCGGGGAAAT |
| | | GTCCGCGGAACCCCTATTTGTTTATTTTTC |
| | | TAAATACATTCAAATATGTATCCGCTCATG |
| | | AGACAATAACCCTGATAAATGCTTCAATAA |
| | | TATTGAAAAAGGAAGAGTATGAGTATTCAA |
| | | CATTTCCGTGTCGCCCTTATTCCCTTTTTT |
| | | GCGGCATTTTGCCTTCCTGTTTTTGCTCAC |
| | | CCAGAAACGCTGGTGAAAGTAAAAGATGCT |
| | | GAAGATCAGTTGGGTGCACGAGTGGGTTAC |
| | | ATCGAACTGGATCTCAACAGCGGTAAGATC |
| | | CTTGAGAGTTTTCGCCCCGAAGAACGTTTT |
| | | CCAATGATGAGCACTTTTAAAGTTCTGCTA |
| | | TGTGGCGCGGTATTATCCCGTGTTGACGCC |
| | | GGGCAAGAGCAACTCGGTCGCCGCATACAC |
| | | TATTCTCAGAATGACTTGGTTGAGTACTCA |
| | | CCAGTCACAGAAAAGCATCTTACGGATGGC |
| | | ATGACAGTAAGAGAATTATGCAGTGCTGCC |
| | | ATAACCATGAGTGATAACACTGCGGCCAAC |
| | | TTACTTCTGACAACGATCGGAGGACCGAAG |
| | | GAGCTAACCGCTTTTTTGCACAACATGGGG |
| | | GATCATGTAACTCGCCTTGATCGTTGGGAA |
| | | CCGGAGCTGAATGAAGCCATACCAAACGAC |
| | | GAGCGTGACACCACGATGCCTGCAGCAATG |
| | | GCAACAACGTTGCGCAAACTATTAACTGGC |
| | | GAACTACTTACTCTAGCTTCCCGGCAACAA |
| | | TTAATAGACTGGATGGAGGCGGATAAAGTT |
| | | GCAGGACCACTTCTGCGCTCGGCCCTTCCG |
| | | GCTGGCTGGTTTATTGCTGATAAATCTGGA |
| | | GCCGGTGAGCGTGGGTCTCGCGGTATCATT |
| | | GCAGCACTGGGGCCAGATGGTAAGCCCTCC |
| | | CGTATCGTAGTTATCTACACGACGGGGAGT |
| | | CAGGCAACTATGGATGAACGAAATAGACAG |
| | | ATCGCTGAGATAGGTGCCTCACTGATTAAG |
| | | CATTGGTAACTGTCAGACCAAGTTTACTCA |
| | | TATATACTTTAGATTGATTTAAAACTTCAT |
| | | TTTTAATTTAAAAGGATCTAGGTGAAGATC |
| | | CTTTTTGATAATCTCATGACCAAAATCCCT |
| | | TAACGTGAGTTTTCGTTCCACTGAGCGTCA |
| | | GACCCCTTAATAAGATGATCTTCTTGAGAT |
| | | CGTTTTGGTCTGCGCGTAATCTCTTGCTCT |
| | | GAAAACGAAAAAACCGCCTTGCAGGGAGGT |
| | | TTTTCGAAGGTTCTCTGAGCTACCAACTCT |
| | | TTGAACCGAGGTAACTGGCTTGCAGGAGCG |
| | | CAGTCACCAAAACTTGTCCTTTCAGTTTAG |
| | | CCTTAACCGGCGCATGACTTCAAGACTAAC |
| | | TCCTCTAAATCAATTACCAGTGGCTGCTGC |
| | | CAGTGGTGCTTTTGCATGTCTTTCCGGGTT |
| | | GGACTCAAGACGATAGTTACCGGATAAGGC |
| | | GCAGCGGTCGGACTGAACGGGGGGTTCGTG |
| | | CATACAGTCCAGCTTGGAGCGAACTGCCTA |
| | | CCCGGAACTGAGTGTCAGGCGTGGAATGAG |
| | | ACAAACGCGGCCATAACAGCGGAATGACAC |
| | | CGGTAAACCGAAAGGCAGGAACAGGAGAGC |
| | | GCACGAGGGAGCCGCCAGGGGGAAACGCCT |
| | | GGTATCTTTATAGTCCTGTCGGGTTTCGCC |
| | | ACCACTGATTTGAGCGTCAGATTTCGTGAT |
| | | GCTTGTCAGGGGGCGGAGCCTATGGAAAAA |
| | | CGCTTTGCCC |
| 24 | FLAG tag | DYKDDDDK |
| 25 | FLAG tag | GACTACAAAGACGATGACGACAAG |

1. A recombinant nucleic acid encoding a fusion protein, wherein the recombinant nucleic acid comprises:
a) a first nucleic acid sequence encoding a spore outer coat polypeptide;
b) a second nucleic acid sequence encoding a linker; and
c) a third nucleic acid sequence encoding a putative insecticidal polypeptide.

2. The recombinant nucleic acid of claim 1, wherein the spore outer coat polypeptide is from a Bacillus bacteria.

3. The recombinant nucleic acid of claim 1, wherein the spore outer coat polypeptide is CotC, CotG, CotB, CotA, CotD, CotE, CotX, CotY, or CotZ.

4. The recombinant nucleic acid of claim 1, wherein the spore outer coat polypeptide is CotC or CotG.

5. The recombinant nucleic acid sequence of claim 1, wherein the putative insecticidal polypeptide is a Bacillus thuringiensis crystal (Bt Cry) protein.

6. The recombinant nucleic acid of claim 5, wherein the Bt Cry protein comprises at least one modification relative to the wild type protein.

7. The recombinant nucleic acid of claim 6, wherein the modification is a result of DNA shuffling, block swapping, site-directed mutagenesis, saturation mutagenesis, random mutagenesis, or a combination thereof.

8. The recombinant nucleic acid of claim 6, wherein the Bt Cry protein modification results in resistance to serine protease digestion.

9. The recombinant nucleic acid of claim 1, wherein the putative insecticidal polypeptide interacts with a cell surface protein of an insect digestive system epithelial cell.

10. The recombinant nucleic acid of claim 9, wherein the cell surface protein is an ATP-Binding Cassette (ABC) transporter protein, a cadherin protein, an aminopeptidase N protein, or an alkaline phosphatase protein.

11. The recombinant nucleic acid of claim 9, wherein the cell surface protein is an ABC transporter protein.

12. The recombinant nucleic acid of claim 10, wherein the ABC transporter protein is PxABCC2, Bm-ABCC2, Sf-ABCC2, Sf-ABCC3, Dv-ABCB1, Bm-ABCB1, Sf-ABCB1, Bm-ABCA2, or Tc-ABCC4.

13. The recombinant nucleic acid of claim 10, wherein the ABC transporter protein is PxABCC2, Bm-ABCC2, Sf-ABCC2, or Sf-ABCC3.

14. The recombinant nucleic acid of claim 1, wherein the spore outer coat polypeptide and the putative insecticidal polypeptide are structurally isolated by the linker.

15. The recombinant nucleic acid of claim 1, wherein the linker comprises at least one amino acid.

16. The recombinant nucleic acid of claim 1, wherein the linker is a polypeptide comprising at least two amino acids.

17. The recombinant nucleic acid of claim 1, wherein the linker is structurally flexible.

18. The recombinant nucleic acid of claim 1, wherein the linker is resistant to protease digestion.

19. The recombinant nucleic acid of claim 1, wherein the linker is a polypeptide comprising an amino acid sequence having at least 80% identity to any of SEQ ID NOs: 20, 21, or 38-61.

20. A DNA construct comprising a promoter operably linked to the recombinant nucleic acid of claim 1.

21. A vector comprising the recombinant nucleic acid of claim 1.

22. A fusion protein encoded by the recombinant nucleic acid of claim 1.

23. A spore comprising the recombinant nucleic acid of claim 1.

24. The spore of claim 23, wherein the fusion protein is displayed on the surface of the spore.

25. The spore of claim 23, wherein the spore is a bacterial spore or a fungal spore.

26. A composition comprising a plurality of the spores of claim 23, wherein each spore in the plurality comprises a recombinant nucleic acid encoding a different putative insecticidal polypeptide.

27. A method of identifying an insecticidal protein, the method comprising:
a) contacting the fusion protein of claim 22 with an insecticidal protein receptor; and
b) detecting an interaction between the fusion protein or spore and the insecticidal protein receptor;
wherein said interaction indicates that the putative insecticidal protein of the fusion protein or spore is an insecticidal protein.

28. The method of claim 27, wherein the insecticidal protein receptor is a purified insecticidal protein receptor protein.

29. The method of claim 27, wherein the insecticidal protein receptor is part of an SMA lipid particle.

30. The method of claim 27, wherein the insecticidal protein receptor is expressed on a cell.

31. The method of claim 30, wherein the cell is an insect cell.

32. The method of claim 27, wherein the insecticidal protein receptor is an ABC transporter protein.

33. The method of claim 27, wherein detecting the interaction between the fusion protein or spore and the insecticidal protein receptor comprises performing immuno-magnetic separation, flow cytometry, cytotoxicity assays, sequencing, or a combination thereof.

34. The method of claim 33, wherein detecting the interaction between the fusion protein or spore and the insecticidal protein receptor protein further comprises isolating the recombinant nucleic acid encoding the fusion protein that interacts with the insecticidal protein receptor protein and performing sequencing to determine the identity of the insecticidal protein.
